(19) United States
(12) Patent Application Publication
(10) Pub. No.: US 2023/0312561 A1
(43) Pub. Date: Oct. 5, 2023

(54) SOLID FORMS OF (Z)-METHYL 3-(((4-(N-METHYL-2-(4-METHYLPIPERAZIN-1-YL)ACETAMIDO)PHENYL)AMINO)(PHENYL)METHYLENE)-2-OXO-2,3-DIHYDRO-1H-PYRROLO[2,3-B]PYRIDINE-6-CARBOXYLATE AND SALTS THEREOF

(71) Applicant: Angion Biomedica Corp., Uniondale, NY (US)

(72) Inventors: Marc W. Andersen, Raleigh, NC (US); Khawla Abdullah Abu-Izza, Berkeley, CA (US)

(21) Appl. No.: 18/011,868

(22) PCT Filed: Jun. 30, 2021
(86) PCT No.: PCT/US2021/039838

§ 371 Date: Dec. 21, 2022

Related U.S. Application Data
(60) Provisional application No. 63/046,919, filed on Jul. 1, 2020.

(51) Int. Cl. C07D 471/04

(52) U.S. Cl. CPC C07D 471/04; C07B 2200/13; A61K 9/48

(57) ABSTRACT

The present disclosure provides crystalline solid forms of an antifibrotic agent, pharmaceutical compositions comprising the same, and methods of use thereof.

### RELATED APPLICATIONS

This application claims priority to U.S. Provisional Patent Application No. 63/046,919, filed Jul. 1, 2020, the entire contents of which are hereby incorporated by reference.

### BACKGROUND

Numerous diseases and conditions responsible for significant morbidity as well as mortality have as an underlying disease mechanism the inappropriate or excessive production of fibrous connective tissue, a process generally known as fibrosis. Such diseases and conditions include fibrotic liver disease, cirrhosis, cardiac fibrosis and lung fibrosis including idiopathic pulmonary fibrosis. In addition to these, numerous other conditions and diseases exhibit a fibrotic component, including but not limited to hepatic ischemia-reperfusion injury, cerebral infarction, ischemic heart disease, heart failure and renal disease including renal fibrosis. These conditions and diseases extract a major toll on the health of afflicted individuals and on the health care system. Means to affect the onset or progression of such conditions and diseases would be highly desirable.

### SUMMARY

Provided herein are novel salt and solid forms, and compositions thereof, useful as antifibrotic agents. In general, provided salt forms and free base forms, and pharmaceutically acceptable compositions thereof, are useful for treating or lessening the severity of a variety of diseases and disorders as described in detail herein.

### BRIEF DESCRIPTION OF THE DRAWINGS

FIG. 1 depicts a composite of the X-ray powder diffraction (XRPD) patterns for each of Compound 1 Hydrochloride Form A (Type 1 Lot), Compound 1 Hydrochloride Form B, Compound 1 Hydrochloride Form C, Compound 1 Hydrochloride Form D, Compound 1 Hydrochloride Form E, Compound 1 Hydrochloride Form F, Compound 1 Hydrochloride Form G, Compound 1 Hydrochloride Form H, and Compound 1 Hydrochloride Form I.

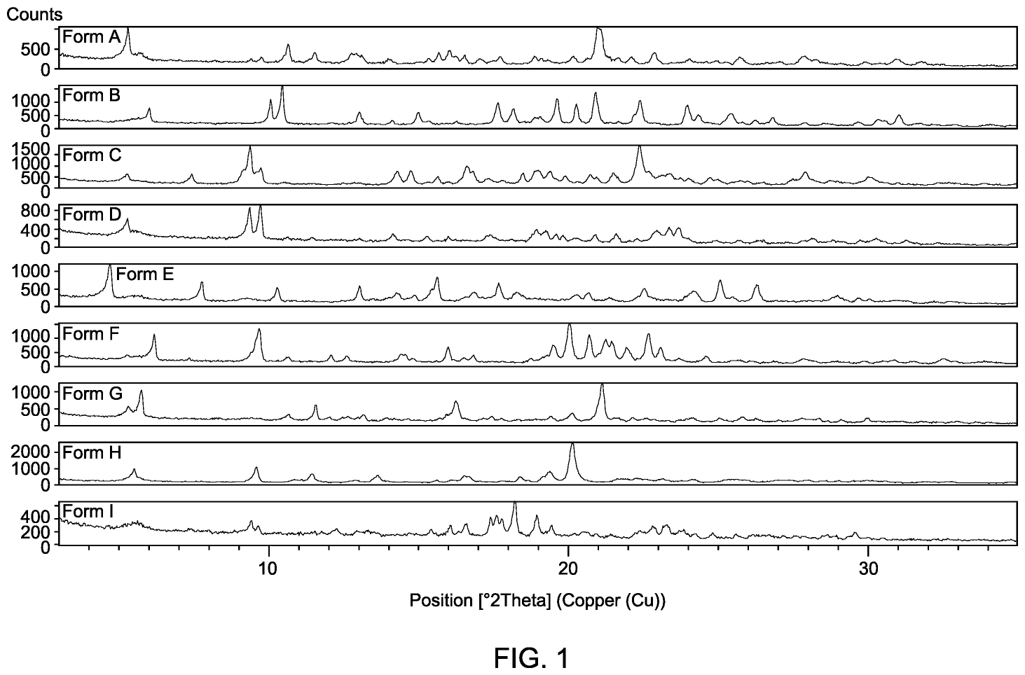

FIG. 2 depicts an XRPD pattern of Compound 1 Hydrochloride Form A (Type 1 Lot).

FIG. 3 depicts a Differential Scanning calorimetry (DSC) curve (A) and Thermogravimetric Analysis (TGA) curve (B) of Compound 1 Hydrochloride Form A (Type 1 Lot).

FIG. 4 depicts a Dynamic Vapor Sorption (DVS) analysis of Compound 1 Hydrochloride Form A (Type 1 Lot).

FIG. 5 depicts an XRPD pattern of Compound 1 Hydrochloride Form A (Type 2 Lot).

FIG. 6 depicts a DSC curve (A) and TGA curve (B) of Compound 1 Hydrochloride Form A (Type 2 Lot).

FIG. 7 depicts an XRPD pattern of Compound 1 Hydrochloride Form B.

FIG. 8 depicts a DSC curve (A) and TGA curve (B) of Compound 1 Hydrochloride Form B.

FIG. 9 depicts an XRPD pattern of Compound 1 Hydrochloride Form C.

FIG. 10 depicts a DSC curve (A) and TGA curve (B) of Compound 1 Hydrochloride Form C.

FIG. 11 depicts an XRPD pattern of Compound 1 Hydrochloride Form D.

FIG. 12 depicts a DSC curve (A) and TGA curve (B) of Compound 1 Hydrochloride Form D.

FIG. 13 depicts an XRPD pattern of Compound 1 Hydrochloride Form E.

FIG. 14 depicts a DSC curve (A) and TGA curve (B) of Compound 1 Hydrochloride Form E.

FIG. 15 depicts an XRPD pattern of Compound 1 Hydrochloride Form F.

FIG. 16 depicts a DSC curve (A) and TGA curve (B) of Compound 1 Hydrochloride Form F.

FIG. 17 depicts an XRPD pattern of Compound 1 Hydrochloride Form G.

FIG. 18 depicts a DSC curve (A) and TGA curve (B) of Compound 1 Hydrochloride Form G.

FIG. 19 depicts an XRPD pattern of Compound 1 Hydrochloride Form H.

FIG. 20 depicts a DSC curve (A) and TGA curve (B) of Compound 1 Hydrochloride Form H.

FIG. 21 depicts an XRPD pattern of Compound 1 Hydrochloride Form I.

FIG. 22 depicts a DSC curve (A) and TGA curve (B) of Compound 1 Hydrochloride Form I.

FIG. 23 depicts an XRPD pattern of amorphous Compound 1 Hydrochloride.

FIG. 24 depicts a DSC curve (A) and TGA curve (B) of amorphous Compound 1 Hydrochloride.

FIG. 25 depicts an XRPD pattern of Compound 1 Free Base Form A.

FIG. 26 depicts a DSC curve (A) and TGA curve (B) of amorphous Compound 1 Free Base.

FIG. 27 depicts an XRPD pattern of Compound 1 Free Base Form B following equilibration of amorphous Compound 1 in methanol.

FIG. 28 depicts a flow chart of solid forms of Compound 1 Hydrochloride obtained from a solvent slurry experiment.

FIG. 29 depicts a schematic of polymorphism of Compound 1 Hydrochloride.

FIG. 30 depicts a DVS analysis of Compound 1 Hydrochloride Form B.

FIG. 31 depicts a DVS analysis of Compound 1 Hydrochloride Form C.

FIG. 32 depicts a DVS analysis of Compound 1 Hydrochloride Form A.

FIG. 33 depicts a DVS analysis of Compound 1 Hydrochloride Form C.

FIG. 34 depicts an XRPD pattern of Compound 1 Maleate Form A.

FIG. 35 depicts a DSC curve of Compound 1 Maleate Form A.

FIG. 36 depicts an XRPD pattern of Compound 1 Mesylate Form A.

FIG. 37 depicts a DSC curve of Compound 1 Mesylate Form A.

FIG. 38 depicts an XRPD pattern of Compound 1 Tosylate Form A.

FIG. 39 depicts a DSC curve of Compound 1 Tosylate Form A.

### DETAILED DESCRIPTION

### Compound 1

PCT Application No. PCT/US2013/023324, filed on Jan. 26, 2013 and published as WO 2013/112959 on Aug. 1, 2013 (“the '324 application”), the entire contents of which is hereby incorporated by reference herein, describes certain antifibrotic compounds. Such compounds include Compound 1:

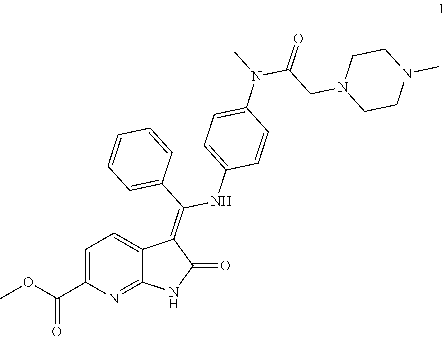

Compound 1 is in a pharmacological class of tyrosine kinase inhibitors (TKI). Compound 1 is an orally bioavailable small molecule dual kinase inhibitor of platelet-derived growth factor receptors (PDGFR) and vascular endothelial growth factor receptors (VEGFR2). Compound 1 is useful in methods provided herein, e.g., for treating or lessening the severity of a disease or disorder mediated by a tyrosine kinase such as PDGFR and/or VEGFR2.

A synthesis of Compound 1, i.e., methyl (Z)-3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate, is described in detail in Example 1 of WO 2013/112959, as well as in Example 1 herein.

Compound 1 is active in a variety of assays and therapeutic models demonstrating antifibrotic activity. Accordingly, Compound 1 and its salts are useful for treating one or more disorders associated with activity of PDGF and/or VEGF.

It would be desirable to provide a form (e.g., a salt and/or solid form) of Compound 1 that, as compared to amorphous Compound 1, imparts characteristics such as improved aqueous solubility, stability, and ease of formulation. Accordingly, the present disclosure provides several salts and/or solid forms of Compound 1.

### Solid Forms of Compound 1

In some embodiments, the present disclosure provides a solid form of Compound 1. In some embodiments, this disclosure provides one or more polymorphic solid forms of Compound 1.

In some embodiments, the present disclosure provides an anhydrous (i.e., unsolvated) polymorphic form of Compound 1.

In some embodiments, the present disclosure provides a crystalline solid form of Compound 1. In some embodiments, the present disclosure provides a composition comprising a crystalline solid form of Compound 1.

### Form A

In some embodiments, a crystalline solid form of Compound 1 is Form A (also referred to herein as Compound 1 Free Base Form A or Compound 1 Form A).

In some embodiments, Compound 1 Form A is characterized by one or more peaks in its XRPD pattern selected from those at about 9.12, 12.31, 17.58, 18.12, and 19.19 degrees 2-theta. In some embodiments, Compound 1 Form A is characterized by two or more peaks in its XRPD pattern selected from those at about 9.12, 12.31, 17.58, 18.12, and 19.19 degrees 2-theta. In some embodiments, Compound 1 Form A is characterized by three or more peaks in its XRPD pattern selected from those at about 9.12, 12.31, 17.58, 18.12, and 19.19 degrees 2-theta.

In some embodiments, Compound 1 Form A is characterized by peaks in its XRPD pattern at about 9.12, 12.31, 17.58, 18.12, and 19.19 degrees 2-theta. In some embodiments, Compound 1 Form A is characterized by peaks in its XRPD pattern at about 9.12, 12.31, 17.58, 18.12, and 19.19 degrees 2-theta, corresponding to d-spacing of about 9.70, 7.19, 5.04, 4.90, and 4.63 angstroms, respectively.

In some embodiments, Compound 1 Form A is characterized by substantially all of the peaks in its XRPD pattern selected from

°2θ ± 0.2

5.55
6.81
9.12
10.56
12.31
13.78
15.35
16.54
17.58
18.12
19.19
20.77
22.78
24.34
26.51
29.45
30.24

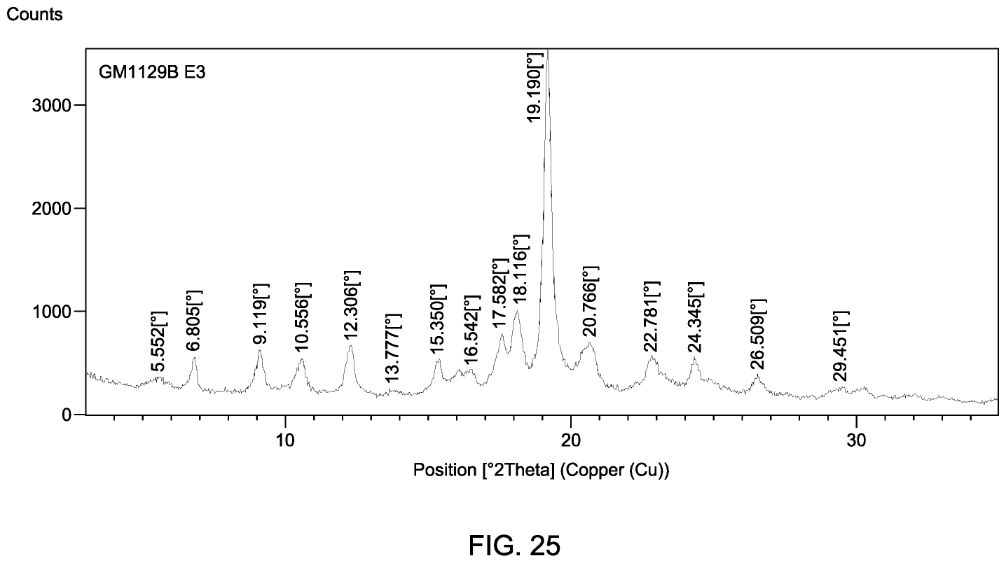

In some embodiments, Compound 1 Form A is characterized by an XRPD pattern substantially similar to that depicted in FIG. 25.

### Form B

In some embodiments, a crystalline solid form of Compound 1 is Form B (also referred to herein as Compound 1 Free Base Form B or Compound 1 Form B).

In some embodiments, Compound 1 Form B is characterized by one or more peaks in its XRPD pattern selected from those at about 7.69, 13.39, 14.64, 18.98, and 20.93 degrees 2-theta. In some embodiments, Compound 1 Form B is characterized by two or more peaks in its XRPD pattern selected from those at about 7.69, 13.39, 14.64, 18.98, and 20.93 degrees 2-theta. In some embodiments, Compound 1 Form B is characterized by three or more peaks in its XRPD pattern selected from those at about 7.69, 13.39, 14.64, 18.98, and 20.93 degrees 2-theta.

In some embodiments, Compound 1 Form B is characterized by peaks in its XRPD pattern at about 7.69, 13.39, 14.64, 18.98, and 20.93 degrees 2-theta. In some embodiments, Compound 1 Form B is characterized by peaks in its XRPD pattern at about 7.69, 13.39, 14.64, 18.98, and 20.93 degrees 2-theta, corresponding to d-spacing of about 11.50, 6.61, 6.05, 4.68, and 4.24 angstroms, respectively.

In some embodiments, Compound 1 Form B is characterized by substantially all of the peaks in its XRPD pattern selected from

°2θ ± 0.2

5.60
7.69
8.76
9.47
10.96
13.39
14.64
16.25
16.66
16.88
17.41
18.98
19.98
20.93
22.03
23.20
23.79
26.07
27.25
27.87
31.51.

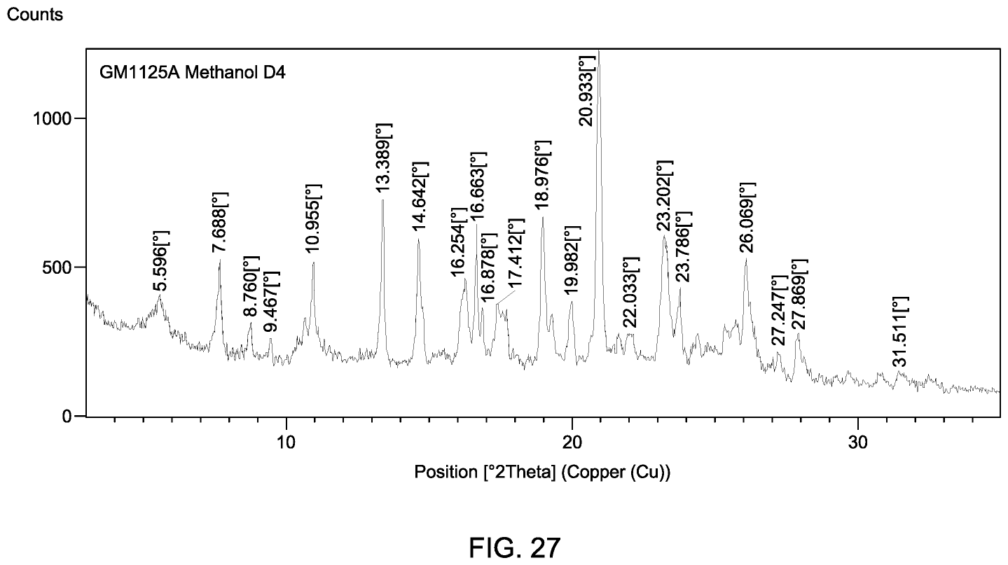

In some embodiments, Compound 1 Form B is characterized by an XRPD pattern substantially similar to that depicted in FIG. 27.

### Salt Forms of Compound 1

In some embodiments, the present disclosure provides a salt of Compound 1, represented by Compound 2:

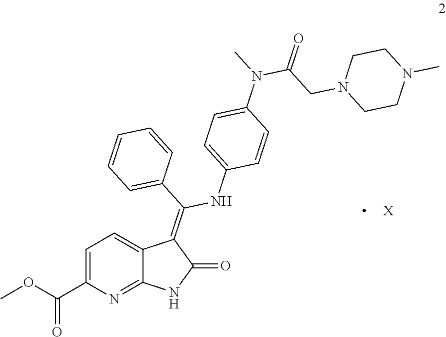

wherein X is hydrochloric acid, maleic acid, methanesulfonic acid, or p-toluenesulfonic acid. In some embodiments, X is hydrochloric acid. In some embodiments, X is maleic acid. In some embodiments, X is methanesulfonic acid. In some embodiments, X is p-toluenesulfonic acid.

It will be appreciated by one of ordinary skill in the art that the acid moiety indicated as “X” and Compound 1 are ionically bonded to form Compound 2. Compound 2 can exist in an amorphous solid form or in a crystalline solid form, or in a mixture thereof. Crystalline solid forms can exist in one or more unique forms, which can be solvates, heterosolvates, hydrates, or unsolvated forms. All such forms are contemplated by the present disclosure.

In some embodiments, this disclosure provides one or more polymorphic solid forms of Compound 2. As used herein, the term “polymorph” refers to the ability of a compound to exist in one or more different crystal structures. For example, one or more polymorphs may vary in pharmaceutically relevant physical properties between one form and another, e.g., solubility, stability, and/or hygroscopicity.

In some embodiments, the present disclosure provides an anhydrous (i.e., unsolvated) polymorphic form of Compound 2.

In some embodiments, the present disclosure provides Compound 2 as a solvate or heterosolvate. As used herein, the term “solvate” refers to a solid form with a stoichiometric or non-stoichiometric amount of one or more solvents incorporated into the crystal structure. For example, a solvated or heterosolvated polymorph can comprise 0.05, 0.1, 0.2, 0.5, 1.0, 1.5, 2.0, etc. equivalents independently of one or more solvents incorporated into the crystal lattice. In some embodiments, a solvate may be a channel solvate.

In some embodiments, Compound 2 is provided as a hydrate. As used herein, the term “hydrate” refers to a solid form with a stoichiometric or non-stoichiometric amount of water incorporated into the crystal structure. For example, a hydrated polymorph can comprise 0.05, 0.1, 0.2, 0.5, 1.0, 1.5, 2.0, etc. equivalents of water incorporated into the crystal lattice. In some embodiments, the hydrate is a channel hydrate.

In some embodiments, Compound 2 is provided as a hydrate and/or a solvate or heterosolvate.

In some embodiments, the present disclosure provides a crystalline solid form of Compound 2. In some embodiments, the present disclosure provides a composition comprising a crystalline solid form of Compound 2.

As used herein, the term “about” when used in reference to a degree 2-theta value refers to the stated value ±0.2 degree 2-theta. In some embodiments, “about” refers to the stated value ±0.1 degree 2-theta.

### Compound 1 Hydrochloride Salt

In some embodiments, the present disclosure provides Compound 1 hydrochloride salts (i.e., Compound 2, wherein X is hydrochloric acid). Compound 1 can exist as at least 9 distinct crystalline hydrochloride salt forms, designated herein as Form A, Form B, Form C, Form D, Form E, Form F, Form G, Form H, and Form I.

### Form A Hydrochloride

In some embodiments, a crystalline solid form of Compound 2 is Compound 1 Hydrochloride Form A. In some embodiments, Compound 1 Hydrochloride Form A is hydrated. In some embodiments, Compound 1 Hydrochloride Form A is a channel hydrate, comprising up to about 4 equivalents of water (e.g., up to about 3 equivalents of water, up to about 2 equivalents of water, or up to about 1 equivalent of water). In some embodiments, Compound 1 Hydrochloride Form A is a channel hydrate, comprising from about 1 to about 4 equivalents of water. In some embodiments, Compound 1 Hydrochloride Form A is a channel hydrate, comprising about 1, about 2, about 3, or about 4 equivalents of water.

The present disclosure also encompasses the recognition that it may desirable for purposes of formulation, stability, storage, and/or manufacturing to provide a Compound 1 Hydrochloride Form A channel hydrate with from about 8% to about 13% water. Accordingly, in some embodiments, a Compound 1 Hydrochloride Form A channel hydrate comprises from about 8 wt % to about 13 wt % water. In some embodiments, a Compound 1 Hydrochloride Form A channel hydrate comprises about 8.5 wt % water. In some embodiments, a Compound 1 Hydrochloride Form A channel hydrate comprises about 10 wt % water. In some embodiments, a Compound 1 Hydrochloride Form A channel hydrate comprises about 13.4 wt % water.

In some embodiments, Compound 1 Hydrochloride Form A is characterized by one or more peaks in its XRPD pattern selected from those at about 5.28, 10.63, 11.54, 17.05, and 20.98 degrees 2-theta. In some embodiments, Compound 1 Hydrochloride Form A is characterized by two or more peaks in its XRPD pattern selected from those at about 5.28, 10.63, 11.54, 17.05, and 20.98 degrees 2-theta. In some embodiments, Compound 1 Hydrochloride Form A is characterized by three or more peaks in its XRPD pattern selected from those at about 5.28, 10.63, 11.54, 17.05, and 20.98 degrees 2-theta.

In some embodiments, Compound 1 Hydrochloride Form A is characterized by peaks in its XRPD pattern at about 5.28, 10.63, 11.54, 17.05, and 20.98 degrees 2-theta. In some embodiments, Compound 1 Hydrochloride Form A is characterized by peaks in its XRPD pattern at about 5.28, 10.63, 11.54, 17.05, and 20.98 degrees 2-theta, corresponding to d-spacing of about 16.74, 8.33, 7.67, 5.20, and 4.23 angstroms, respectively.

In some embodiments, Compound 1 Hydrochloride Form A is characterized by substantially all of the peaks in its XRPD pattern selected from

°2θ ± 0.2

5.28
5.76
10.63
11.54
12.73
13.13
14.08
15.34
15.64
16.00
16.55
17.05
17.78
18.86
19.08
20.16
20.68
20.98
21.62
22.05
22.82
23.97
24.94
25.23
25.61
26.58
27.01
27.78
29.89
30.51
30.91
31.72
33.39.

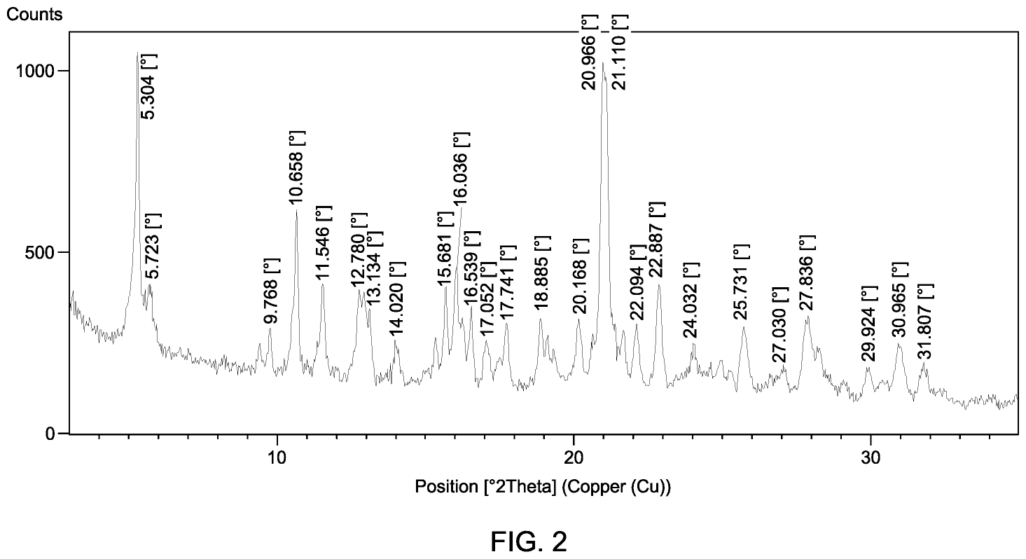

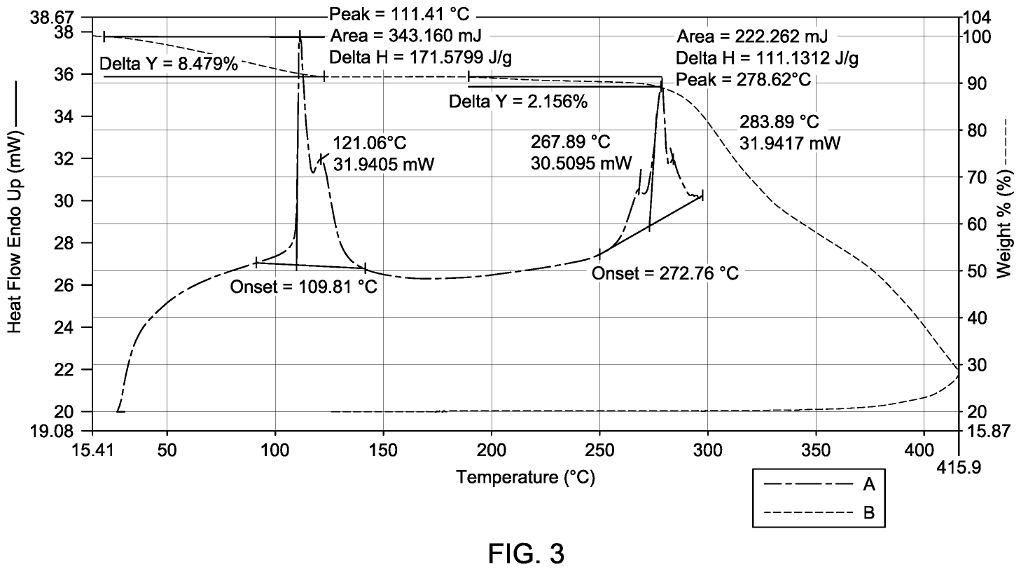

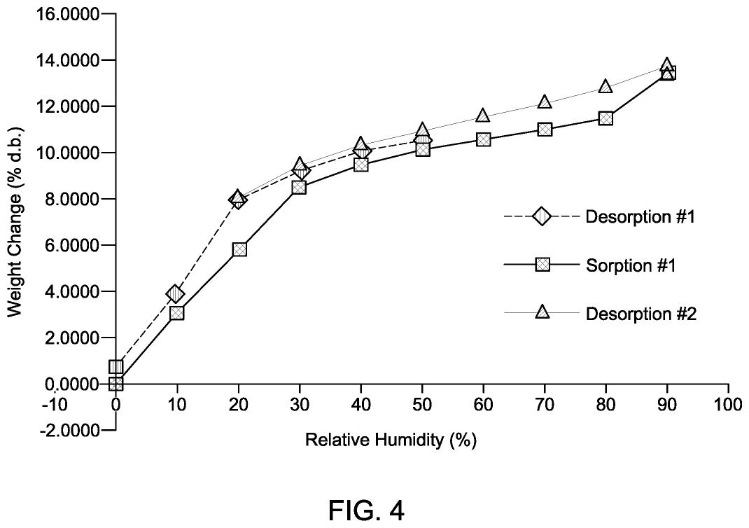

In some embodiments, Compound 1 Hydrochloride Form A is characterized by one or more of the following:
(i) an XRPD pattern substantially similar to that depicted in FIG. 2;
(ii) a TGA pattern substantially similar to that depicted in FIG. 3;
(iii) a DSC pattern substantially similar to that depicted in FIG. 3; and
(iv) a DVS pattern substantially similar to that depicted in FIG. 4.

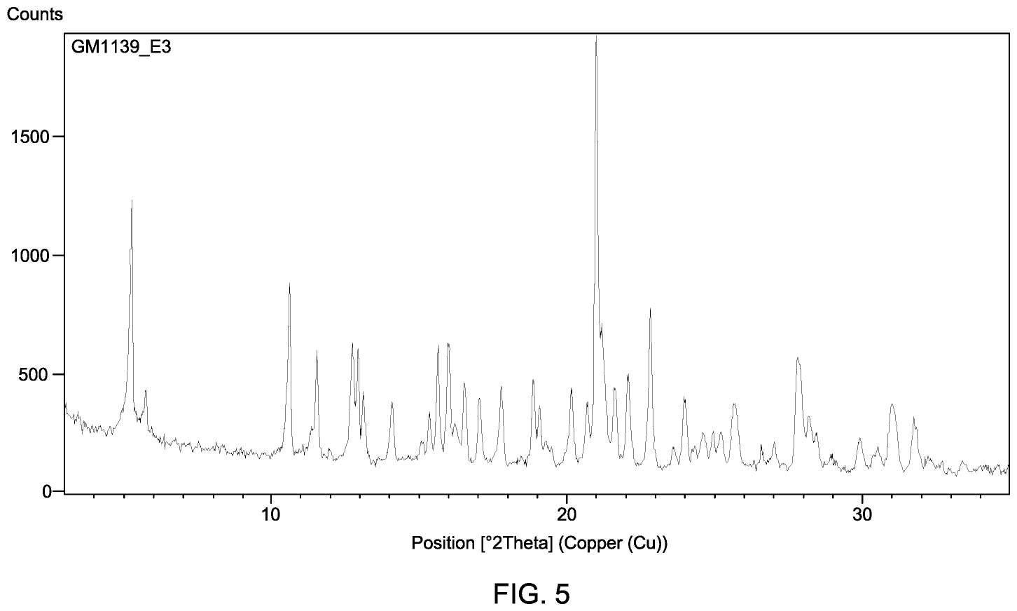

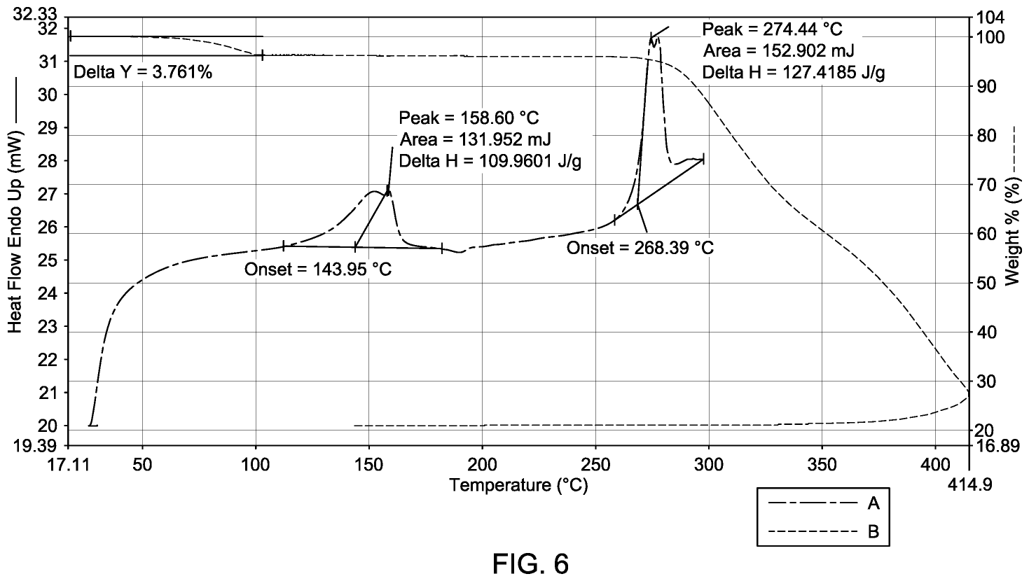

In some embodiments, Compound 1 Hydrochloride Form A is characterized by one or more of the following:
(i) an XRPD pattern substantially similar to that depicted in FIG. 5;
(ii) a TGA pattern substantially similar to that depicted in FIG. 6; and
(iii) a DSC pattern substantially similar to that depicted in FIG. 6.

### Form B Hydrochloride

In some embodiments, a crystalline solid form of Compound 2 is Compound 1 Hydrochloride Form B. In some embodiments, Compound 1 Hydrochloride Form B is hydrated. In some embodiments, Compound 1 Hydrochloride Form B is a monohydrate.

In some embodiments, Compound 1 Hydrochloride Form B is characterized by one or more peaks in its XRPD pattern selected from those at about 6.02, 10.07, 10.46, 13.02, and 14.99 degrees 2-theta. In some embodiments, Compound 1 Hydrochloride Form B is characterized by two or more peaks in its XRPD pattern selected from those at about 6.02, 10.07, 10.46, 13.02, and 14.99 degrees 2-theta. In some embodiments, Compound 1 Hydrochloride Form B is characterized by three or more peaks in its XRPD pattern selected from those at about 6.02, 10.07, 10.46, 13.02, and 14.99 degrees 2-theta.

In some embodiments, Compound 1 Hydrochloride Form B is characterized by peaks in its XRPD pattern at about 6.02, 10.07, 10.46, 13.02, and 14.99 degrees 2-theta. In some embodiments, Compound 1 Hydrochloride Form B is characterized by peaks in its XRPD pattern at about 6.02, 10.07, 10.46, 13.02, and 14.99 degrees 2-theta, corresponding to d-spacing of about 14.68, 8.78, 8.46, 6.80, and 5.91 angstroms, respectively.

In some embodiments, Compound 1 Hydrochloride Form B is characterized by substantially all of the peaks in its XRPD pattern selected from

°2θ ± 0.2

6.02
10.07
10.46
13.02
14.13
14.99
15.35
16.27
17.65
18.17
18.88
19.63
20.27
20.90
22.41
23.96
24.37
25.45
26.22
26.82
27.90
28.61
29.66
30.30
31.04
31.72
32.91
34.13.

In some embodiments, Compound 1 Hydrochloride Form B is characterized by one or more of the following:

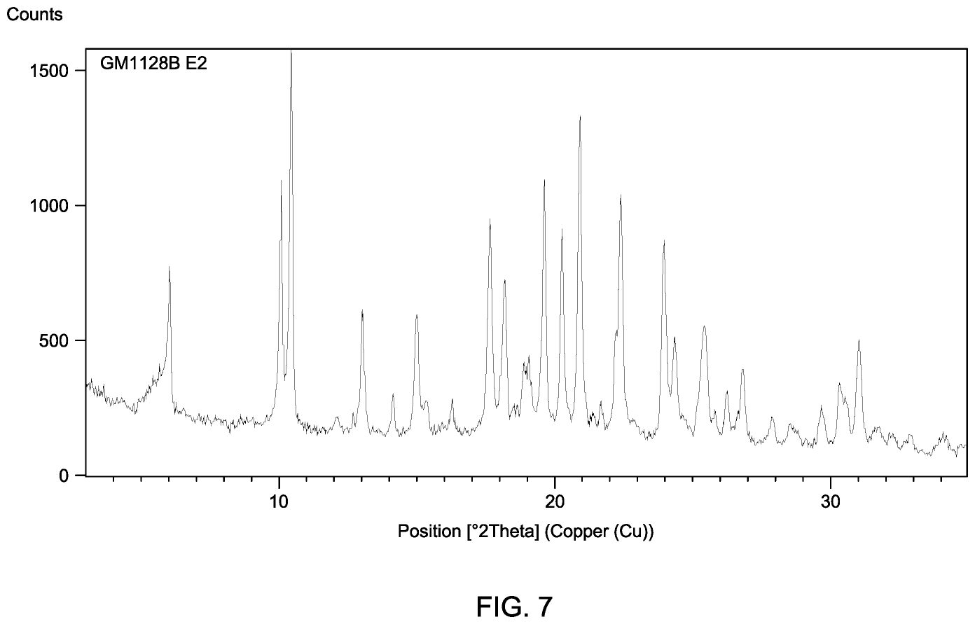

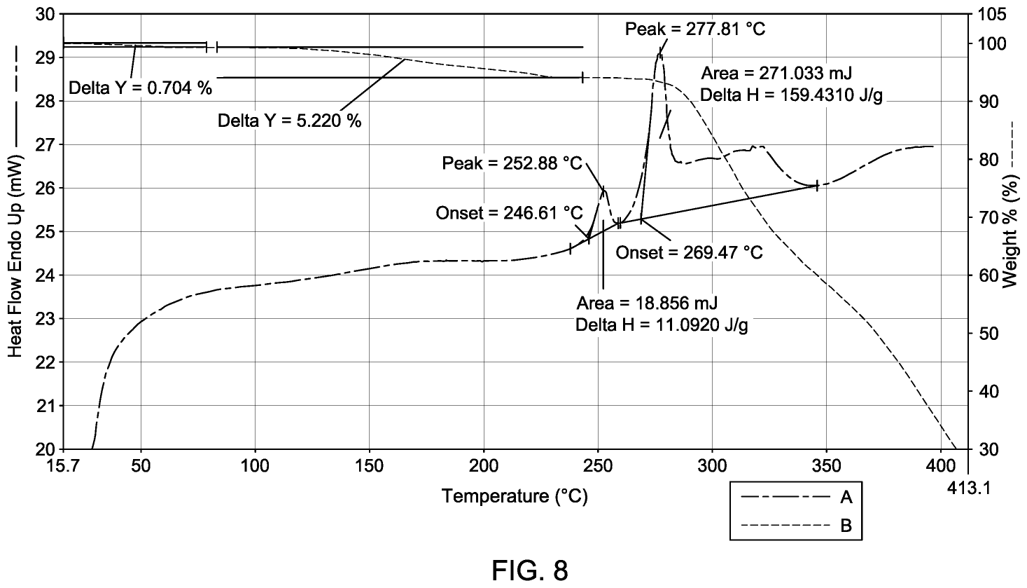

an XRPD pattern substantially similar to that depicted in FIG. 7;
(ii) a TGA pattern substantially similar to that depicted in FIG. 8; and
(iii) a DSC pattern substantially similar to that depicted in FIG. 8.

### Form C Hydrochloride

In some embodiments, a crystalline solid form of Compound 2 is Compound 1 Hydrochloride Form C. In some embodiments, Compound 1 Hydrochloride Form C is anhydrous (i.e., unsolvated).

In some embodiments, Compound 1 Hydrochloride Form C is characterized by one or more peaks in its XRPD pattern selected from those at about 5.29, 7.44, 9.38, 14.29, and 14.76 degrees 2-theta. In some embodiments, Compound 1 Hydrochloride Form C is characterized by two or more peaks in its XRPD pattern selected from those at about 5.29, 7.44, 9.38, 14.29, and 14.76 degrees 2-theta. In some embodiments, Compound 1 Hydrochloride Form C is characterized by three or more peaks in its XRPD pattern selected from those at about 5.29, 7.44, 9.38, 14.29, and 14.76 degrees 2-theta.

In some embodiments, Compound 1 Hydrochloride Form C is characterized by peaks in its XRPD pattern at about 5.29, 7.44, 9.38, 14.29, and 14.76 degrees 2-theta. In some embodiments, Compound 1 Hydrochloride Form C is characterized by peaks in its XRPD pattern at about 5.29, 7.44, 9.38, 14.29, and 14.76 degrees 2-theta, corresponding to d-spacing of about 16.70, 11.88, 9.43, 6.20, and 6.00 angstroms, respectively.

In some embodiments, Compound 1 Hydrochloride Form C is characterized by substantially all of the peaks in its XRPD pattern selected from

°2θ ± 0.2

5.29
7.44
9.38
9.75
10.51
12.97
14.29
14.76
15.66
16.61
16.84
17.26
17.81
18.48
18.87
19.05
19.41
19.90
20.72
21.50
22.38
23.44
24.01
24.73
25.85
26.50
27.44
27.93
28.74
30.04
30.99
32.41
33.89.

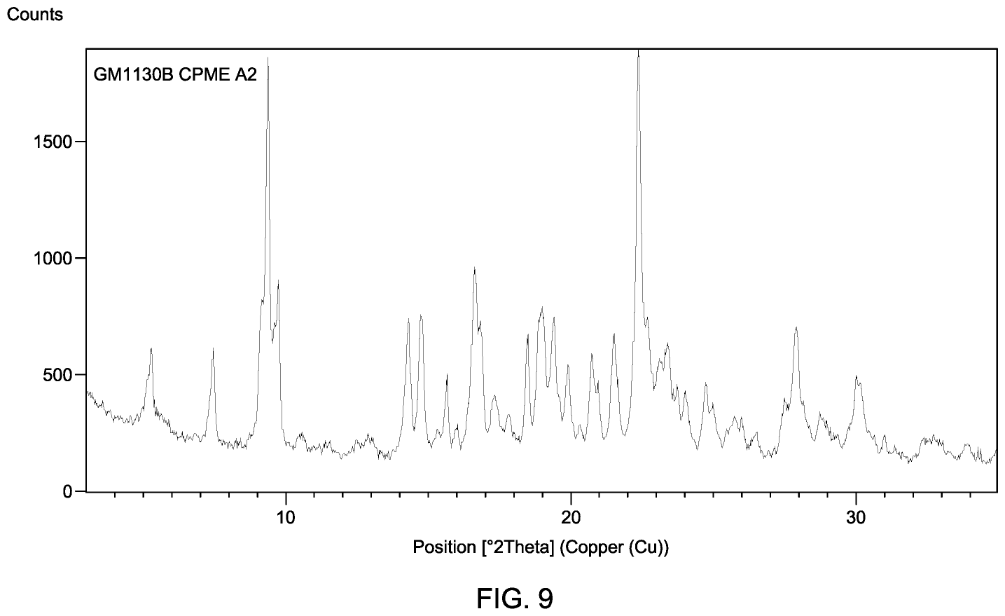

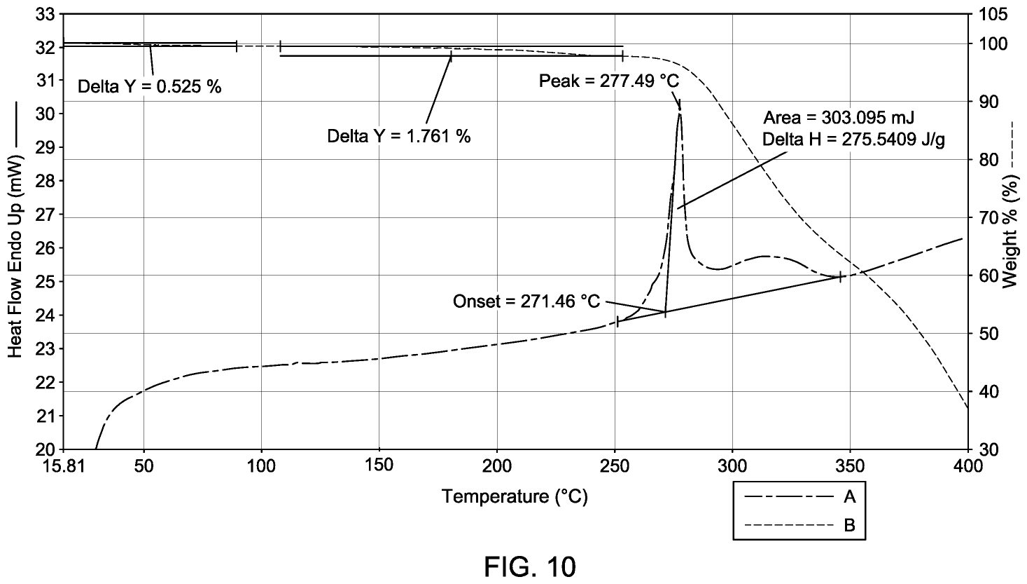

In some embodiments, Compound 1 Hydrochloride Form C is characterized by one or more of the following:
(i) an XRPD pattern substantially similar to that depicted in FIG. 9;
(ii) a TGA pattern substantially similar to that depicted in FIG. 10; and
(iii) a DSC pattern substantially similar to that depicted in FIG. 10.

### Form D Hydrochloride

In some embodiments, a crystalline solid form of Compound 2 is Compound 1 Hydrochloride Form D. In some embodiments, Compound 1 Hydrochloride Form D is hydrated. In some embodiments, Compound 1 Hydrochloride Form D is a dihydrate.

In some embodiments, Compound 1 Hydrochloride Form D is characterized by one or more peaks in its XRPD pattern selected from those at about 5.29, 9.37, 9.73, 15.29, 21.60, and 23.71 degrees 2-theta. In some embodiments, Compound 1 Hydrochloride Form D is characterized by two or more peaks in its XRPD pattern selected from those at about 5.29, 9.37, 9.73, 15.29, 21.60, and 23.71 degrees 2-theta. In some embodiments, Compound 1 Hydrochloride Form D is characterized by three or more peaks in its XRPD pattern selected from those at about 5.29, 9.37, 9.73, 15.29, 21.60, and 23.71 degrees 2-theta.

In some embodiments, Compound 1 Hydrochloride Form D is characterized by peaks in its XRPD pattern at about 5.29, 9.37, 9.73, 15.29, 21.60, and 23.71 degrees 2-theta. In some embodiments, Compound 1 Hydrochloride Form D is characterized by peaks in its XRPD pattern at about 5.29, 9.37, 9.73, 15.29, 21.60, and 23.71 degrees 2-theta, corresponding to d-spacing of about 16.72, 9.44, 9.09, 5.80, 4.12, and 3.75 angstroms, respectively.

In some embodiments, Compound 1 Hydrochloride Form D is characterized by substantially all of the peaks in its XRPD pattern selected from

°2θ ± 0.2

5.29
9.37
9.73
14.16
15.29
15.99
17.35
18.94
19.30
19.59
19.82
20.26
20.89
21.60
22.99
23.39
23.71
24.94
25.70
26.50
28.23
28.96
30.28
31.27
32.37.

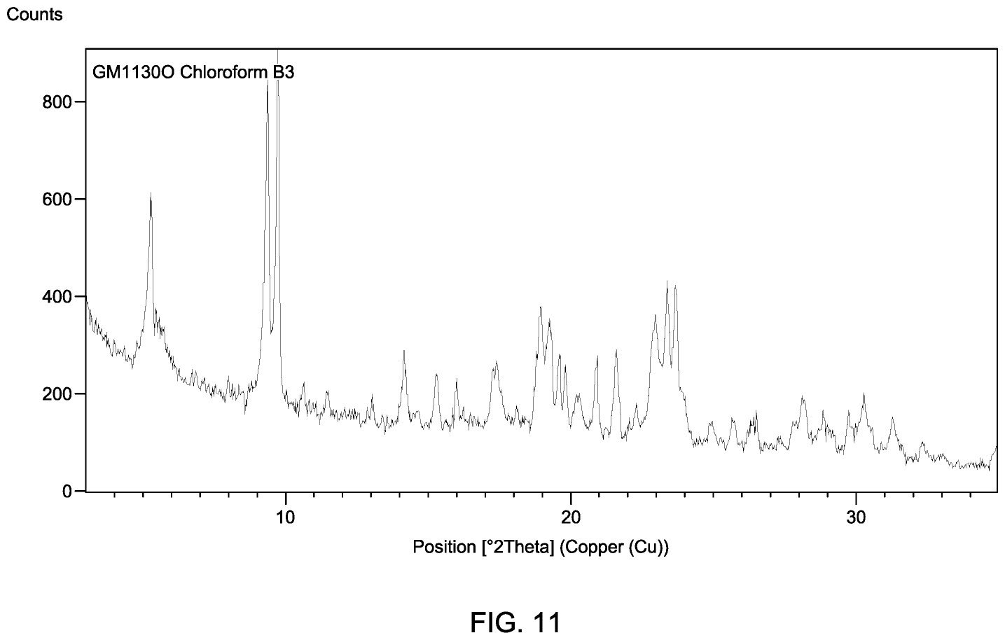

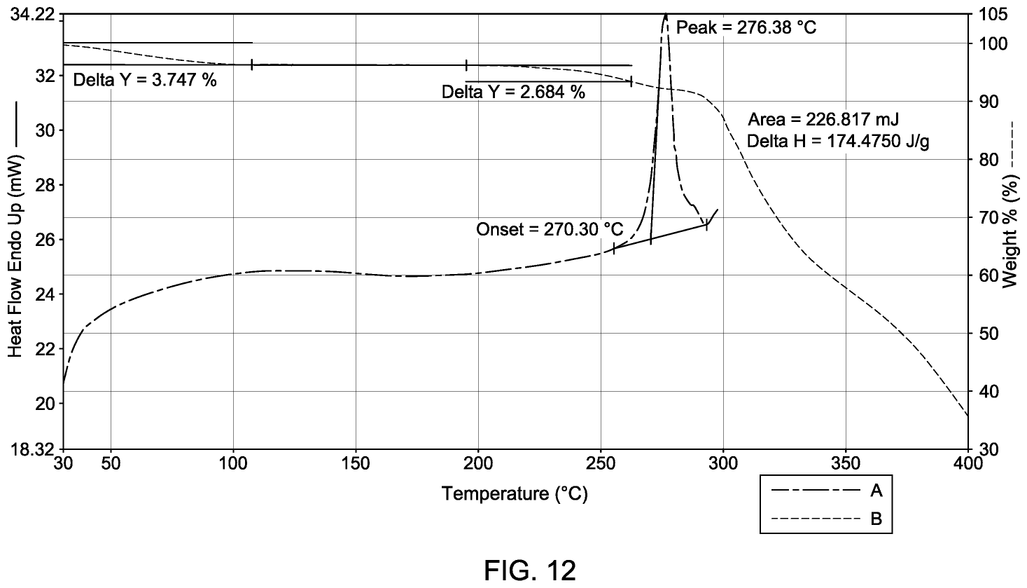

In some embodiments, Compound 1 Hydrochloride Form D is characterized by one or more of the following:
(i) an XRPD pattern substantially similar to that depicted in FIG. 11;
(ii) a TGA pattern substantially similar to that depicted in FIG. 12; and
(iii) a DSC pattern substantially similar to that depicted in FIG. 12.

### Form E Hydrochloride

In some embodiments, a crystalline solid form of Compound 2 is Compound 1 Hydrochloride Form E. In some embodiments, Compound 1 Hydrochloride Form E is hydrated. In some embodiments, Compound 1 Hydrochloride Form E is a dihydrate.

In some embodiments, Compound 1 Hydrochloride Form E is characterized by one or more peaks in its XRPD pattern selected from those at about 4.71, 7.78, 10.30, 13.04, and 15.64 degrees 2-theta. In some embodiments, Compound 1 Hydrochloride Form E is characterized by two or more peaks in its XRPD pattern selected from those at about 4.71, 7.78, 10.30, 13.04, and 15.64 degrees 2-theta. In some embodiments, Compound 1 Hydrochloride Form E is characterized by three or more peaks in its XRPD pattern selected from those at about 4.71, 7.78, 10.30, 13.04, and 15.64 degrees 2-theta.

In some embodiments, Compound 1 Hydrochloride Form E is characterized by peaks in its XRPD pattern at about 4.71, 7.78, 10.30, 13.04, and 15.64 degrees 2-theta. In some embodiments, Compound 1 Hydrochloride Form E is characterized by peaks in its XRPD pattern at about 4.71, 7.78, 10.30, 13.04, and 15.64 degrees 2-theta, corresponding to d-spacing of about 18.74, 11.37, 8.59, 6.79, and 5.67 angstroms, respectively.

In some embodiments, Compound 1 Hydrochloride Form E is characterized by substantially all of the peaks in its XRPD pattern selected from

°2θ ± 0.2

4.71
5.61
7.78
9.25
10.30
13.04
14.27
14.88
15.64
16.91
17.67
18.26
20.35
20.69
21.37
22.53
24.20
25.06
25.51
26.31
29.00
29.69
31.28
32.77.

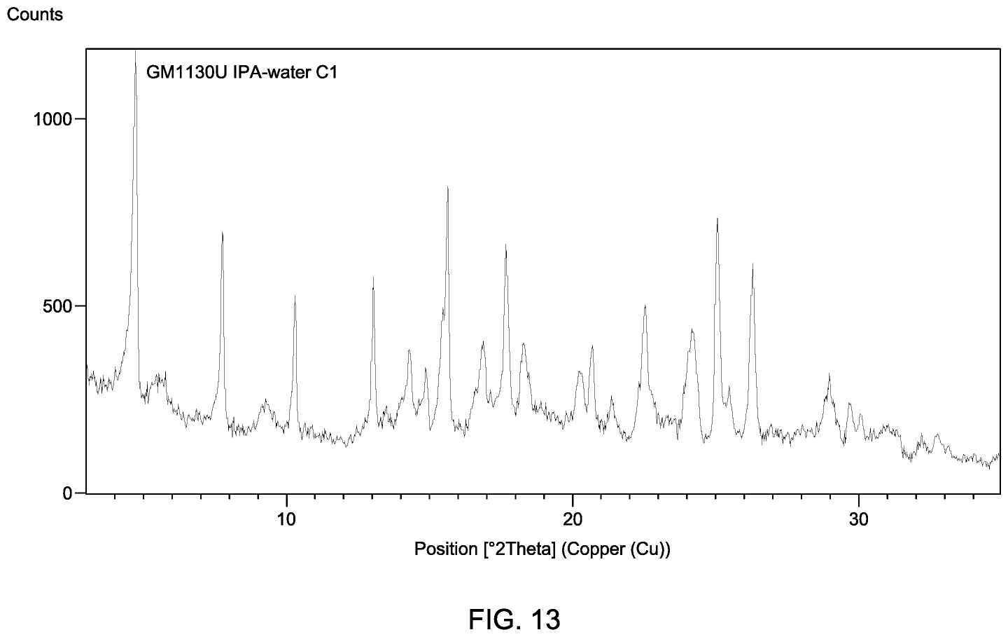

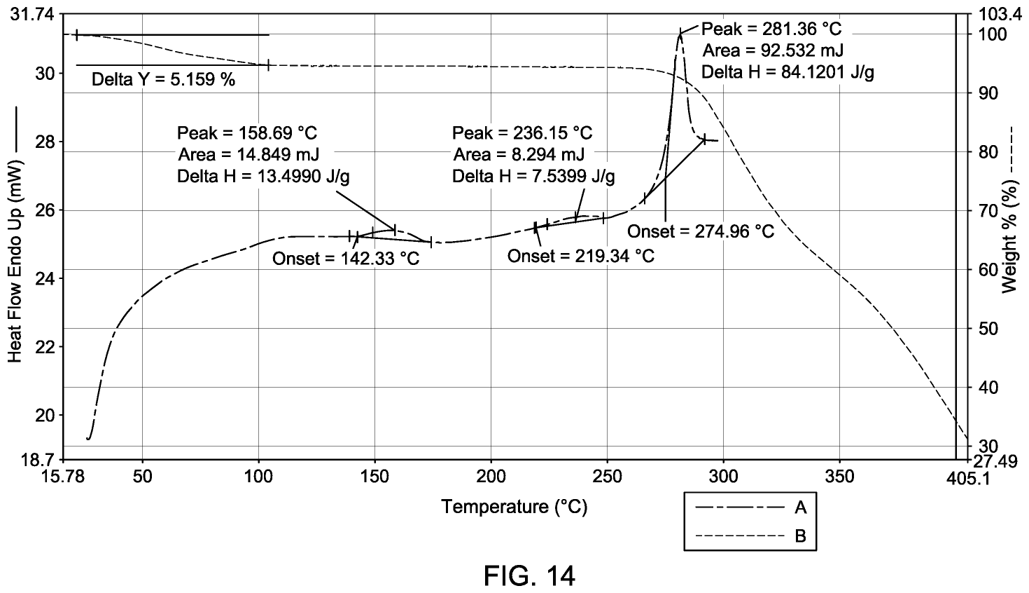

In some embodiments, Compound 1 Hydrochloride Form E is characterized by one or more of the following:
(i) an XRPD pattern substantially similar to that depicted in FIG. 13;
(ii) a TGA pattern substantially similar to that depicted in FIG. 14; and
(iii) a DSC pattern substantially similar to that depicted in FIG. 14.

### Form F Hydrochloride

In some embodiments, a crystalline solid form of Compound 2 is Compound 1 Hydrochloride Form F. In some embodiments, Compound 1 Hydrochloride Form F is anhydrous (i.e., unsolvated).

In some embodiments, Compound 1 Hydrochloride Form F is characterized by one or more peaks in its XRPD pattern selected from those at about 6.19, 9.70, 12.09, 16.00, and 20.05 degrees 2-theta. In some embodiments, Compound 1 Hydrochloride Form F is characterized by two or more peaks in its XRPD pattern selected from those at about 6.19, 9.70, 12.09, 16.00, and 20.05 degrees 2-theta. In some embodiments, Compound 1 Hydrochloride Form F is characterized by three or more peaks in its XRPD pattern selected from those at about 6.19, 9.70, 12.09, 16.00, and 20.05 degrees 2-theta.

In some embodiments, Compound 1 Hydrochloride Form F is characterized by peaks in its XRPD pattern at about 6.19, 9.70, 12.09, 16.00, and 20.05 degrees 2-theta. In some embodiments, Compound 1 Hydrochloride Form F is characterized by peaks in its XRPD pattern at about 6.19, 9.70, 12.09, 16.00, and 20.05 degrees 2-theta, corresponding to d-spacing of about 14.28, 9.12, 7.32, 5.54, and 4.43 angstroms, respectively.

In some embodiments, Compound 1 Hydrochloride Form F is characterized by substantially all of the peaks in its XRPD pattern selected from

°2θ ± 0.2

5.27
6.19
7.30
9.70
10.65
12.09
12.62
14.41
16.00
16.50
16.83
18.73
19.50
20.05
20.71
21.24
21.49
21.95
22.68
23.08
23.71
24.62
25.74
26.89
27.85
28.92
29.88
30.87
31.53
32.49
33.96.

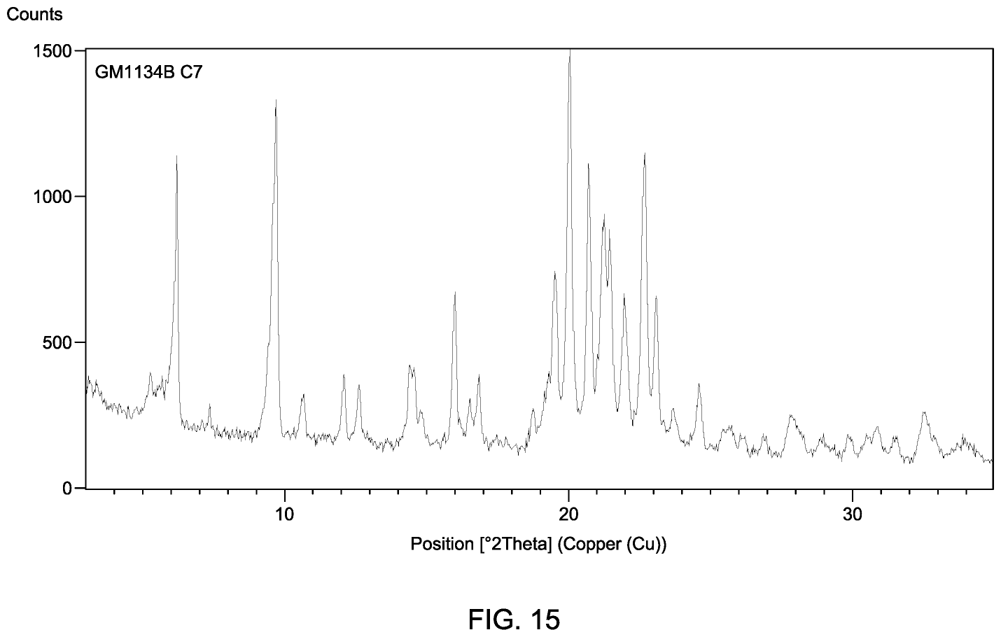

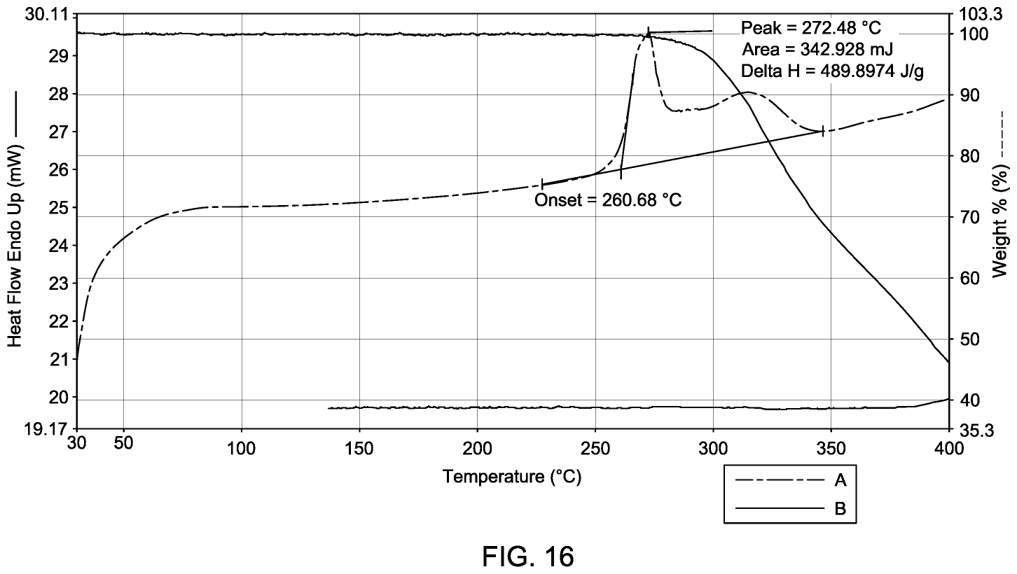

In some embodiments, Compound 1 Hydrochloride Form F is characterized by one or more of the following:
(i) an XRPD pattern substantially similar to that depicted in FIG. 15;
(ii) a TGA pattern substantially similar to that depicted in FIG. 16; and
(iii) a DSC pattern substantially similar to that depicted in FIG. 16.

### Form G Hydrochloride

In some embodiments, a crystalline solid form of Compound 2 is Compound 1 Hydrochloride Form G. In some embodiments, Compound 1 Hydrochloride Form G is hydrated. In some embodiments, Compound 1 Hydrochloride Form G is a trihydrate.

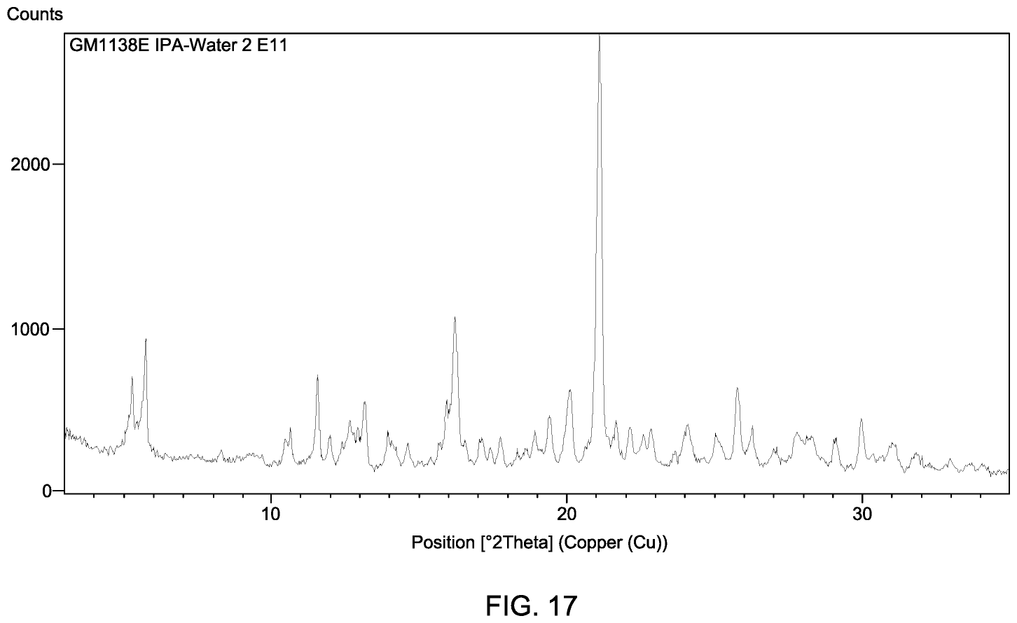

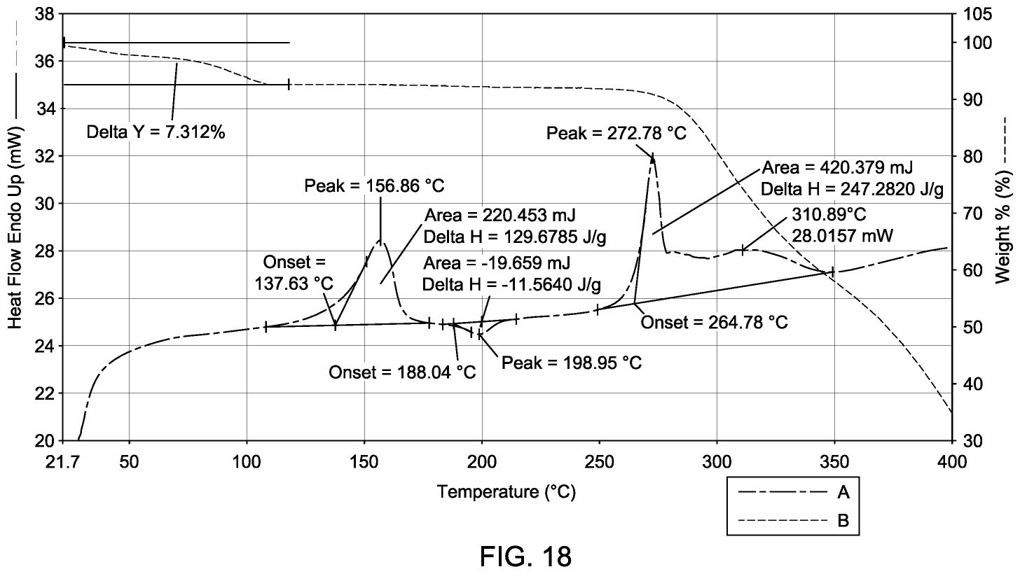

In some embodiments, Compound 1 Hydrochloride Form G is characterized by one or more of the following:
(i) an XRPD pattern substantially similar to that depicted in FIG. 17;
(ii) a TGA pattern substantially similar to that depicted in FIG. 18; and
(iii) a DSC pattern substantially similar to that depicted in FIG. 18.

### Form H Hydrochloride

In some embodiments, a crystalline solid form of Compound 2 is Compound 1 Hydrochloride Form H. In some embodiments, Compound 1 Hydrochloride Form H is hydrated. In some embodiments, Compound 1 Hydrochloride Form H is a trihydrate.

In some embodiments, Compound 1 Hydrochloride Form H is characterized by one or more peaks in its XRPD pattern selected from those at about 5.52, 9.60, 11.46, 13.66, and 20.13 degrees 2-theta. In some embodiments, Compound 1 Hydrochloride Form H is characterized by two or more peaks in its XRPD pattern selected from those at about 5.52, 9.60, 11.46, 13.66, and 20.13 degrees 2-theta. In some embodiments, Compound 1 Hydrochloride Form H is characterized by three or more peaks in its XRPD pattern selected from those at about 5.52, 9.60, 11.46, 13.66, and 20.13 degrees 2-theta.

In some embodiments, Compound 1 Hydrochloride Form H is characterized by peaks in its XRPD pattern at about 5.52, 9.60, 11.46, 13.66, and 20.13 degrees 2-theta. In some embodiments, Compound 1 Hydrochloride Form H is characterized by peaks in its XRPD pattern at about 5.52, 9.60, 11.46, 13.66, and 20.13 degrees 2-theta, corresponding to d-spacing of about 16.00, 9.21, 7.72, 6.48, and 4.41 angstroms, respectively.

In some embodiments, Compound 1 Hydrochloride Form H is characterized by substantially all of the peaks in its XRPD pattern selected from

°2θ ± 0.2

5.52
9.60
10.85
11.46
12.93
13.66
15.60
16.07
16.52
18.39
19.36
20.13
21.60
22.29
23.12
24.16
25.35
26.97
30.03
31.95
32.71.

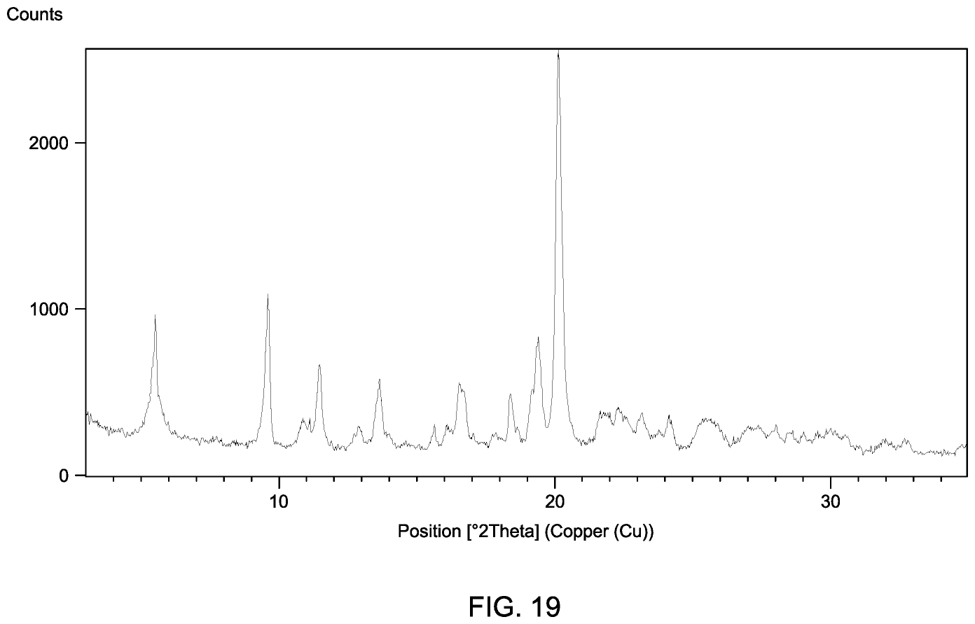

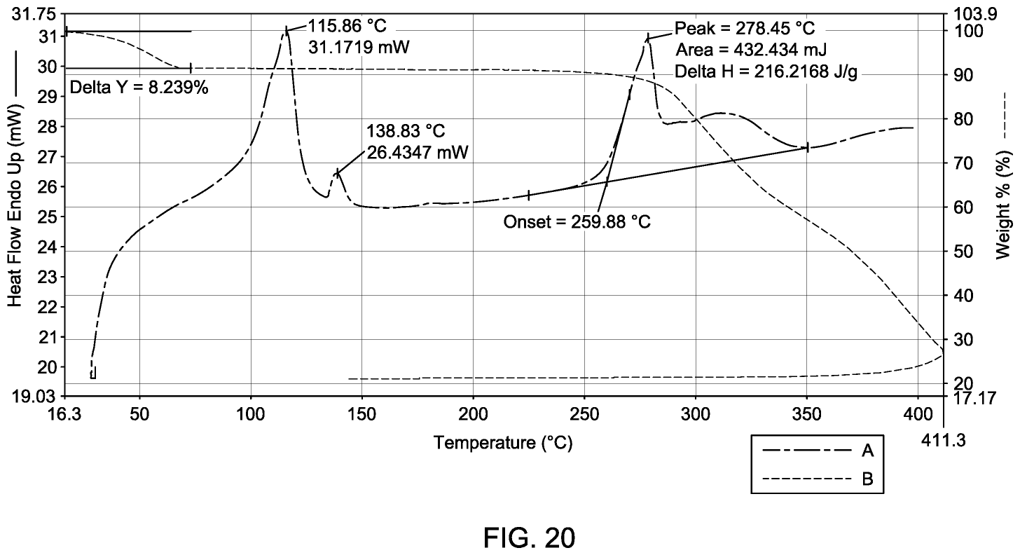

In some embodiments, Compound 1 Hydrochloride Form H is characterized by one or more of the following:
(i) an XRPD pattern substantially similar to that depicted in FIG. 19;
(ii) a TGA pattern substantially similar to that depicted in FIG. 20; and
(iii) a DSC pattern substantially similar to that depicted in FIG. 20.

### Form I Hydrochloride

In some embodiments, a crystalline solid form of Compound 2 is Compound 1 Hydrochloride Form I. In some embodiments, Compound 1 Hydrochloride Form I is anhydrous (i.e., unsolvated).

In some embodiments, Compound 1 Hydrochloride Form I is characterized by one or more peaks in its XRPD pattern selected from those at about 9.42, 12.24, 17.40, 17.60, 17.80, and 18.22 degrees 2-theta. In some embodiments, Compound 1 Hydrochloride Form I is characterized by two or more peaks in its XRPD pattern selected from those at about 9.42, 12.24, 17.40, 17.60, 17.80, and 18.22 degrees 2-theta. In some embodiments, Compound 1 Hydrochloride Form I is characterized by three or more peaks in its XRPD pattern selected from those at about 9.42, 12.24, 17.40, 17.60, 17.80, and 18.22 degrees 2-theta.

In some embodiments, Compound 1 Hydrochloride Form I is characterized by peaks in its XRPD pattern at about 9.42, 12.24, 17.40, 17.60, 17.80, and 18.22 degrees 2-theta. In some embodiments, Compound 1 Hydrochloride Form I is characterized by peaks in its XRPD pattern at about 9.42, 12.24, 17.40, 17.60, 17.80, and 18.22 degrees 2-theta, corresponding to d-spacing of about 9.39, 7.23, 5.10, 5.04, 4.98, and 4.87 angstroms, respectively.

In some embodiments, Compound 1 Hydrochloride Form I is characterized by substantially all of the peaks in its XRPD pattern selected from

°2θ ± 0.2

5.60
9.42
12.24
13.44
15.42
16.06
16.59
17.40
17.60
17.80
18.22
18.95
19.45
20.58
22.85
23.26
23.78
24.24
24.81
25.61
26.48
28.68
29.58.

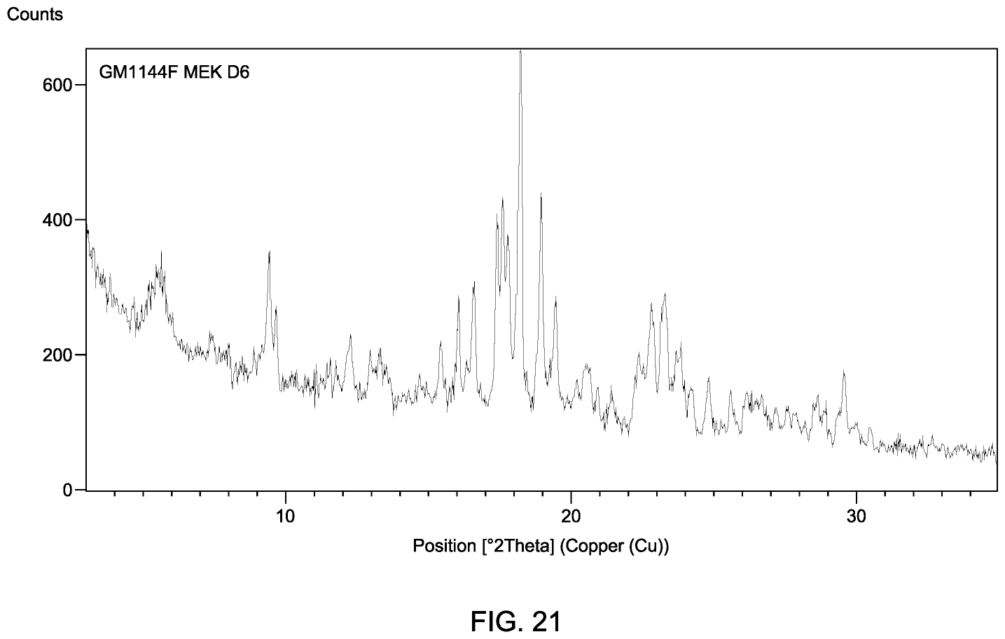

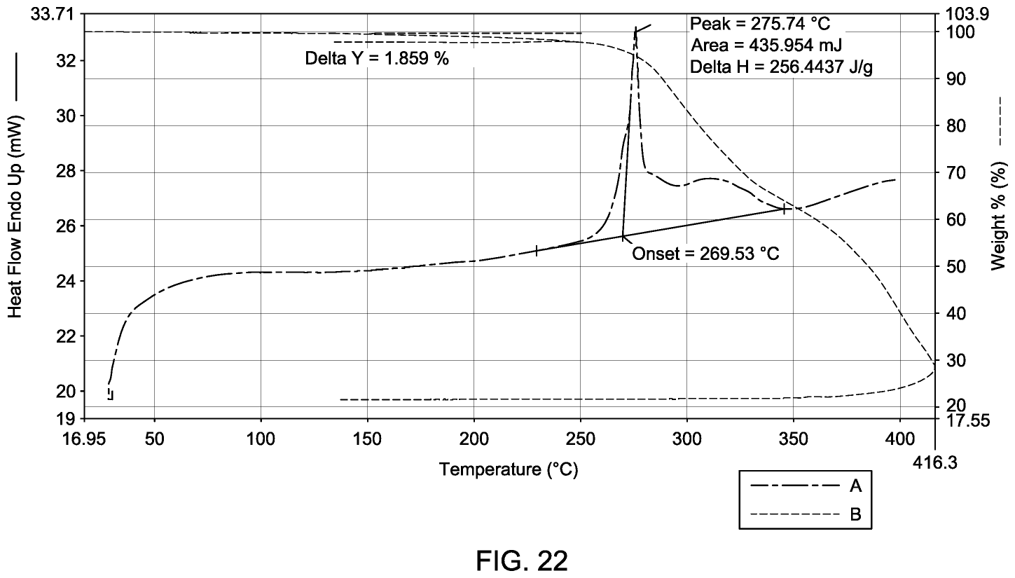

In some embodiments, Compound 1 Hydrochloride Form I is characterized by one or more of the following:
(i) an XRPD pattern substantially similar to that depicted in FIG. 21;
(ii) a TGA pattern substantially similar to that depicted in FIG. 22; and
(iii) a DSC pattern substantially similar to that depicted in FIG. 22.

### Compound 1 Maleate Salt

In some embodiments, the present disclosure provides Compound 1 maleate salts (i.e., Compound 2, wherein X is maleic acid). Compound 1 can exist as at least 1 crystalline maleate salt form, designated herein as Form A (i.e., Compound 1 Maleate Form A). In some embodiments, Compound 1 Maleate Form A is an anhydrate.

In some embodiments, Compound 1 Maleate Form A is characterized by one or more peaks in its XRPD pattern selected from those at about 9.37, 10.10, 11.74, 14.89, 15.67, 15.91, 18.08, and 18.42 degrees 2-theta. In some embodiments, Compound 1 Maleate Form A is characterized by two or more peaks in its XRPD pattern selected from those at about 9.37, 10.10, 11.74, 14.89, 15.67, 15.91, 18.08, and 18.42 degrees 2-theta. In some embodiments, Compound 1 Maleate Form A is characterized by three or more peaks in its XRPD pattern selected from those at about 9.37, 10.10, 11.74, 14.89, 15.67, 15.91, 18.08, and 18.42 degrees 2-theta.

In some embodiments, Compound 1 Maleate Form A is characterized by peaks in its XRPD pattern at about 9.37, 10.10, 11.74, 14.89, 15.67, 15.91, 18.08, and 18.42 degrees 2-theta. In some embodiments, Compound 1 Maleate Form A is characterized by peaks in its XRPD pattern at about 9.37, 10.10, 11.74, 14.89, 15.67, 15.91, 18.08, and 18.42 degrees 2-theta, corresponding to d-spacing of about 9.44, 8.76, 7.54, 5.95, 5.66, 5.57, 4.91, and 4.82 angstroms, respectively.

In some embodiments, Compound 1 Maleate Form A is characterized by substantially all of the peaks in its XRPD pattern selected from

°2θ ± 0.2

5.53
6.35
8.12
9.37
10.10
10.89
11.74
12.61
13.00
14.89
15.67
15.91
16.65
17.58
18.08
18.42
18.82
19.43
19.95
20.92
21.76
22.47
22.90
23.81
24.33
25.30
25.83
26.49
28.37
28.87
29.85
30.65
32.16
34.21.

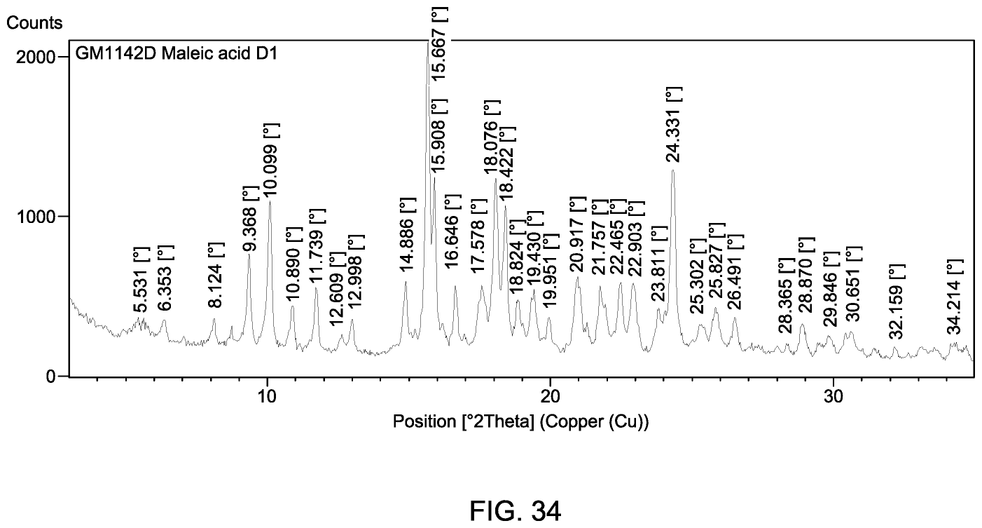

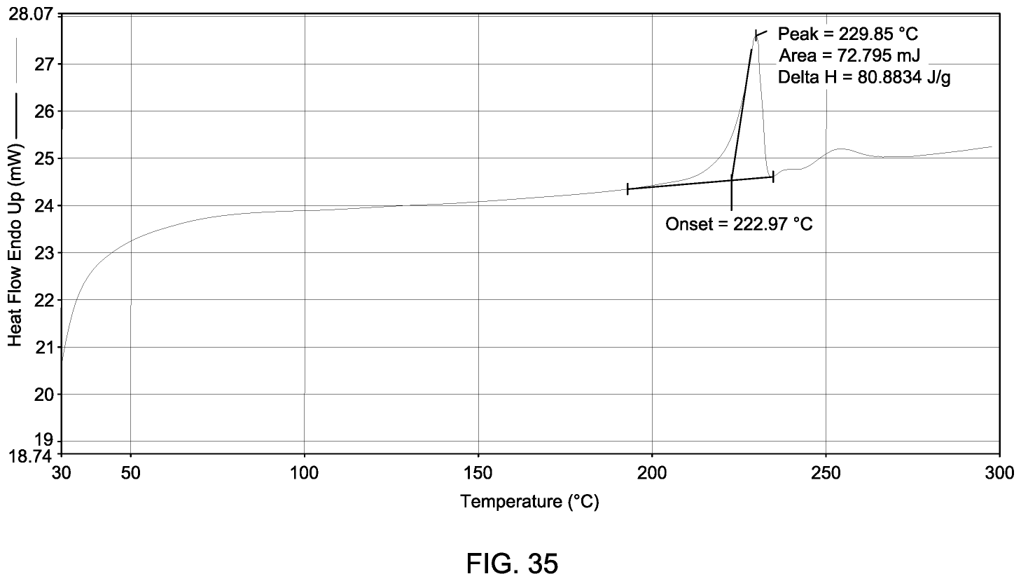

In some embodiments, Compound 1 Maleate Form A is characterized by one or more of the following:
(i) an XRPD pattern substantially similar to that depicted in FIG. 34; and
(ii) a DSC pattern substantially similar to that depicted in FIG. 35.

### Compound 1 Mesylate Salt

In some embodiments, the present disclosure provides Compound 1 mesylate salts (i.e., Compound 2, wherein X is methanesulfonic acid). Compound 1 can exist as at least 1 crystalline mesylate salt form, designated herein as Form A (i.e., Compound 1 Mesylate Form A). In some embodiments, Compound 1 Mesylate Form A is an anhydrate.

In some embodiments, Compound 1 Mesylate Form A is characterized by one or more peaks in its XRPD pattern selected from those at about 9.53, 9.78, 16.47, 17.36, 19.48, and 20.12 degrees 2-theta. In some embodiments, Compound 1 Mesylate Form A is characterized by two or more peaks in its XRPD pattern selected from those at about 9.53, 9.78, 16.47, 17.36, 19.48, and 20.12 degrees 2-theta. In some embodiments, Compound 1 Mesylate Form A is characterized by three or more peaks in its XRPD pattern selected from those at about 9.53, 9.78, 16.47, 17.36, 19.48, and 20.12 degrees 2-theta.

In some embodiments, Compound 1 Mesylate Form A is characterized by peaks in its XRPD pattern at about 9.53, 9.78, 16.47, 17.36, 19.48, and 20.12 degrees 2-theta. In some embodiments, Compound 1 Mesylate Form A is characterized by peaks in its XRPD pattern at about 9.53, 9.78, 16.47, 17.36, 19.48, and 20.12 degrees 2-theta, corresponding to d-spacing of about 9.28, 9.04, 5.38, 5.11, 4.56, and 4.41 angstroms, respectively.

In some embodiments, Compound 1 Mesylate Form A is characterized by substantially all of the peaks in its XRPD pattern selected from

°2θ ± 0.2

5.70
7.75
9.53
9.78
10.55
10.93
11.53
12.07
13.05
13.50
13.75
14.08
14.27
14.96
15.87
16.47
16.79
17.36
17.97
18.84
19.18
19.48
19.68
20.12
20.49
21.39
22.10
22.75
23.28
23.97
24.66
27.09
27.75
29.45
31.30
32.81.

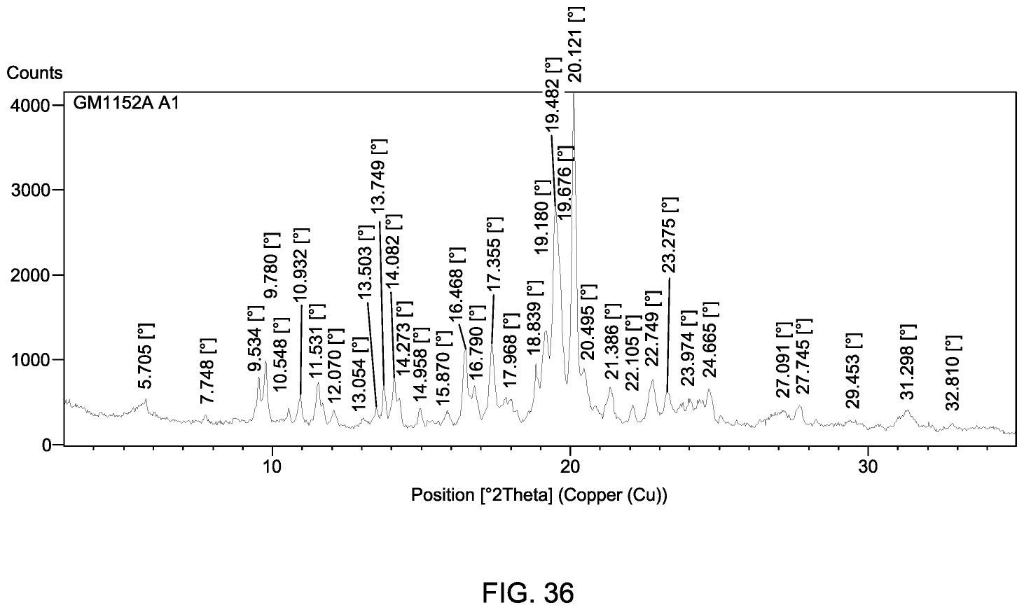

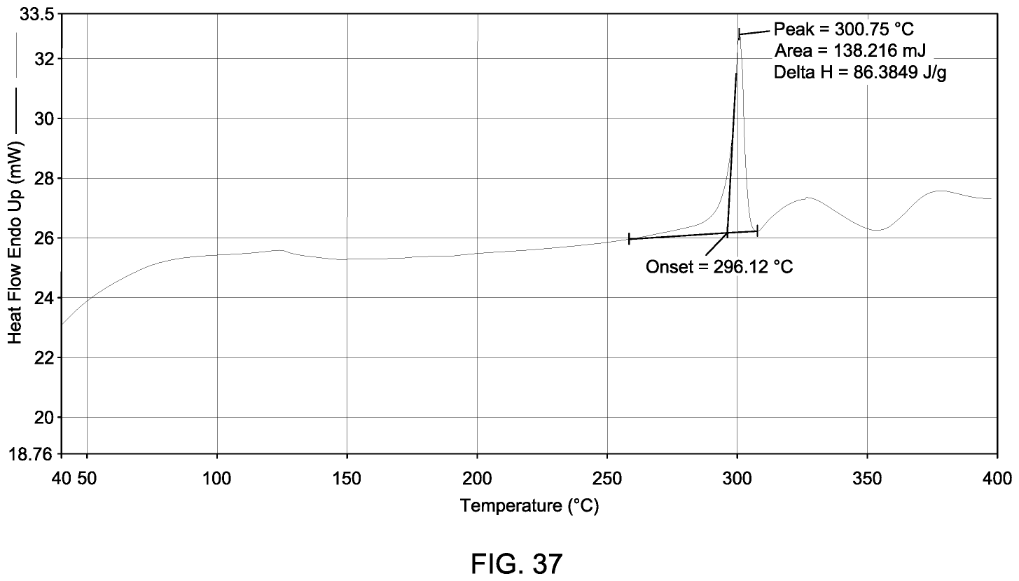

In some embodiments, Compound 1 Mesylate Form A is characterized by one or more of the following:
(i) an XRPD pattern substantially similar to that depicted in FIG. 36; and
(ii) a DSC pattern substantially similar to that depicted in FIG. 37.

### Compound 1 Tosylate Salt

In some embodiments, the present disclosure provides Compound 1 tosylate salts (i.e., Compound 2, wherein X is p-toluenesulfonic acid). Compound 1 can exist as at least 1 crystalline tosylate salt form, designated herein as Form A (i.e., Compound 1 Tosylate Form A). In some embodiments, Compound 1 Tosylate Form A is an anhydrate.

In some embodiments, Compound 1 Tosylate Form A is characterized by one or more peaks in its XRPD pattern selected from those at about 9.72, 10.01, 11.16, 12.81, 17.58, and 18.20 degrees 2-theta. In some embodiments, Compound 1 Tosylate Form A is characterized by two or more peaks in its XRPD pattern selected from those at about 9.72, 10.01, 11.16, 12.81, 17.58, and 18.20 degrees 2-theta. In some embodiments, Compound 1 Tosylate Form A is characterized by three or more peaks in its XRPD pattern selected from those at about 9.72, 10.01, 11.16, 12.81, 17.58, and 18.20 degrees 2-theta.

In some embodiments, Compound 1 Tosylate Form A is characterized by peaks in its XRPD pattern at about 9.72, 10.01, 11.16, 12.81, 17.58, and 18.20 degrees 2-theta. In some embodiments, Compound 1 Tosylate Form A is characterized by peaks in its XRPD pattern at about 9.72, 10.01, 11.16, 12.81, 17.58, and 18.20 degrees 2-theta, corresponding to d-spacing of about 9.10, 8.84, 7.93, 6.91, 5.05, 4.88 angstroms, respectively.

In some embodiments, Compound 1 Tosylate Form A is characterized by substantially all of the peaks in its XRPD pattern selected from

°2θ ± 0.2

6.00
9.72
10.01
11.16
12.18
12.81
13.53
13.95
16.32
16.91
17.58
18.20
18.53
19.44
19.90
20.15
20.66
21.22
21.67
22.35
23.36
23.81
24.09
24.63
25.18
25.42
25.84
26.67
27.32
29.56
31.23
31.95
33.50.

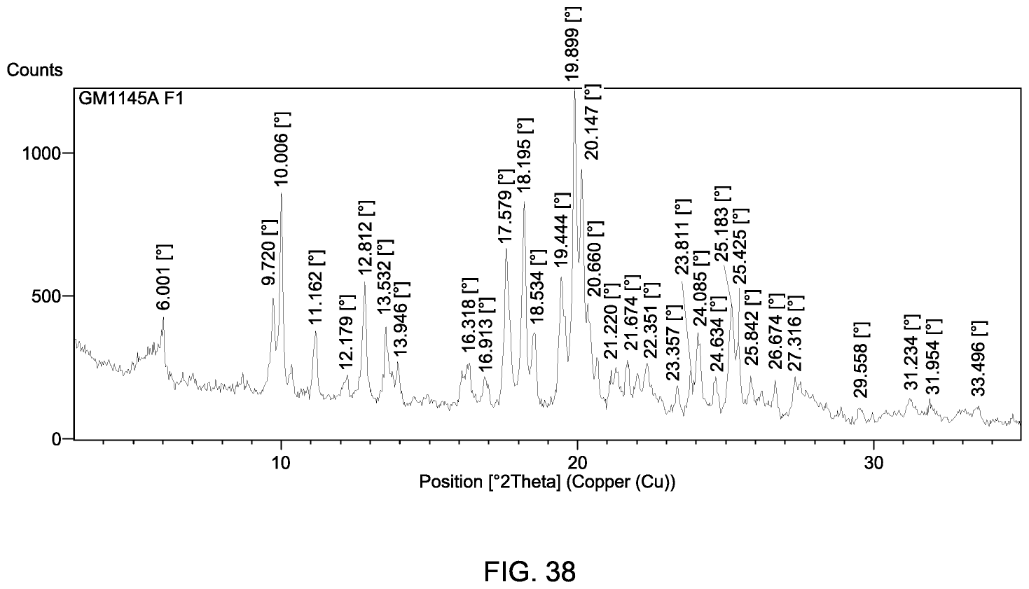

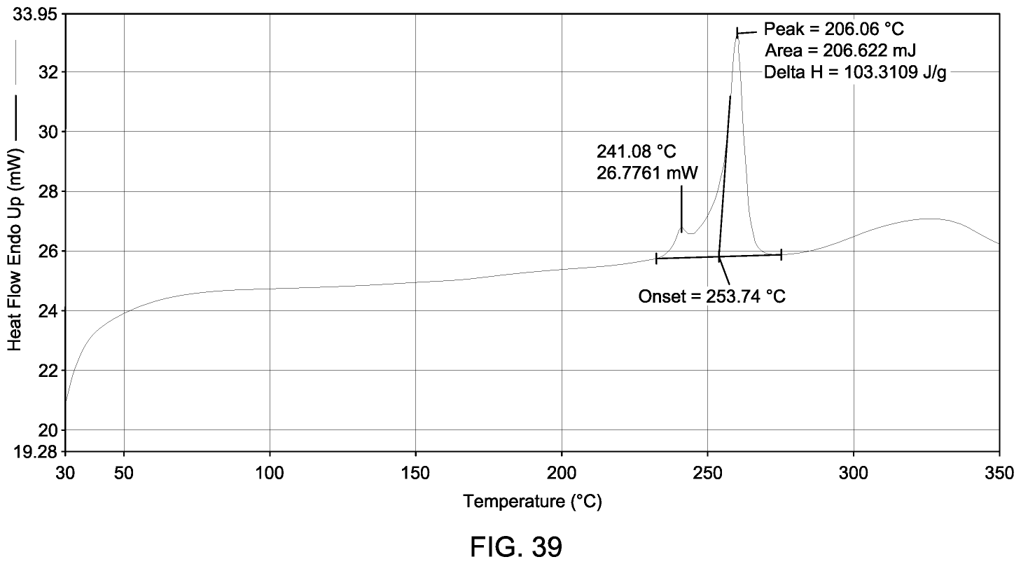

In some embodiments, Compound 1 Tosylate Form A is characterized by one or more of the following:
(i) an XRPD pattern substantially similar to that depicted in FIG. 38; and
(ii) a DSC pattern substantially similar to that depicted in FIG. 39

### Methods of Preparing Provided Salt and Solid Forms

The present disclosure provides methods of preparing Compound 2 (i.e., salt forms of Compound 1), as well as solid forms of Compound 1 and Compound 2. The present disclosure also provides methods of preparing compositions comprising mixtures of Compound 1 and/or Compound 2 in one or more solid forms and/or an amorphous form.

### Compound 1

In some embodiments, Compound 1 (i.e., Compound 1 Free Base) is prepared by contacting Compound 2 (e.g., Compound 1 Hydrochloride, e.g., amorphous Compound 1 Hydrochloride, crystalline Compound 1 Hydrochloride, or a mixture thereof) with a suitable base, such as sodium bicarbonate. In some embodiments, the present disclosure provides a method of preparing Compound 1 comprising steps of providing Compound 2; and combining Compound 2 with a suitable base, optionally in a suitable solvent to provide Compound 1.

In some embodiments, solid forms of Compound 1 (i.e., Compound 1 Free Base) can be prepared by dissolving Compound 1 (e.g., amorphous Compound 1, crystalline Compound 1, or a mixture thereof) in a suitable solvent and then causing Compound 1 to return to the solid phase. In some embodiments, solids forms of Compound 1 are prepared by combining amorphous and/or crystalline Compound 1 in a suitable solvent under suitable conditions and isolating a solid form of Compound 1.

In some embodiments, a suitable solvent is selected from acetonitrile, 1,4-dioxane, ethyl acetate, isopropyl alcohol, methanol, methyl ethyl ketone, methyl isobutyl ketone, 2-methyltetrahydrofuran, tetrahydrofuran, and water, or any combination thereof.

In some embodiments, a method of preparing Compound 1 comprises a step of heating a mixture comprising Compound 1 to a suitable temperature. In some embodiments, a suitable temperature is from about 30° C. to about 60° C. In some embodiments, a method of preparing Compound 1 comprises a step of stirring a mixture comprising Compound 1 at ambient temperature.

In some embodiments, Compound 1 precipitates from a mixture (e.g., a solution, suspension or slurry). In some embodiments, Compound 1 crystallizes from a solution. In some embodiments, Compound 1 crystallizes from a solution following seeding of the solution (e.g., adding crystals of Compound 1 to the solution). In some embodiments, Compound 1 precipitates or crystallizes from a mixture after removal of part or all of a solvent through methods such as evaporation, distillation, filtration, reverse osmosis, absorption or reaction, by adding an anti-solvent, by cooling, or by any combination of such methods.

In some embodiments, a method of preparing Compound 1 comprises a step of isolating Compound 1. It will be appreciated that Compound 1 may be isolated by any suitable means. In some embodiments, Compound 1 (e.g., precipitated or crystallized Compound 1) is separated from a supernatant by filtration. In some embodiments, Compound 1 (e.g., precipitated or crystallized Compound 1) is separated from a supernatant by decanting the supernatant.

In some embodiments, isolated Compound 1 is dried (e.g., in air or under reduced pressure, optionally at elevated temperature).

In some embodiments, a solid form of Compound 1 is prepared by converting one solid form of Compound 1 into another solid form of Compound 1.

### Compound 2

In some embodiments, Compound 2 is prepared by contacting Compound 1 (i.e., Compound 1 Free Base, e.g., amorphous Compound 1 Free Base, crystalline Compound 1 Free Base, or a mixture thereof) with a suitable acid, such as hydrochloric acid, maleic acid, methanesulfonic acid, or 4-toluenesulfonic acid. In some embodiments, the present disclosure provides a method of preparing Compound 2 comprising steps of providing Compound 1; and combining Compound 1 with a suitable acid, optionally in a suitable solvent, to provide Compound 2. In some embodiments, about 1.0, about 1.1, or about 2.0, or more equivalents of a suitable acid are added.

In some embodiments, solid forms of Compound 2 can be prepared by dissolving Compound 2 (e.g., amorphous Compound 2, crystalline Compound 2, or a mixture thereof) in a suitable solvent and then causing Compound 2 to return to the solid phase. In some embodiments, solids forms of Compound 2 are prepared by combining amorphous and/or crystalline Compound 2 in a suitable solvent under suitable conditions and isolating a solid form of Compound 2.

In some embodiments, a suitable solvent is selected from acetone, acetonitrile, anisole, 2-chlorobutane, chloroform, cumene, cyclohexane, cyclopentyl methyl ether, dichloromethane, 1,4-dioxane, ethanol, ethyl acetate, heptane, isopropyl acetate, isopropyl alcohol, methanol, methyl ethyl ketone, methyl isobutyl ketone, 2-methyltetrahydrofuran, nitromethane, tert-butyl methyl ether, tetrahydrofuran, toluene, water, or any combination thereof.

In some embodiments, a method of preparing Compound 2 comprises a step of heating a mixture comprising Compound 2 to a suitable temperature. In some embodiments, a suitable temperature is from about 30° C. to about 60° C. (e.g., about 38° C., about 50° C.).

In some embodiments, a method of preparing Compound 1 comprises a step of stirring a mixture comprising Compound 1 at ambient temperature.

In some embodiments, a mixture comprising Compound 2 is cooled to a suitable temperature. In some embodiments, a suitable temperature is from about 0° C. to about 45° C. (e.g., about 25° C., about 40° C.).

In some embodiments, Compound 2 precipitates from a mixture (e.g., a solution, suspension or slurry). In some embodiments, Compound 2 crystallizes from a solution. In some embodiments, Compound 2 crystallizes from a solution following seeding of the solution (e.g., adding crystals of Compound 2 to the solution). In some embodiments, Compound 2 precipitates or crystallizes from a mixture after removal of part or all of a solvent through methods such as evaporation, distillation, filtration, reverse osmosis, absorption or reaction, by adding an anti-solvent (e.g., isopropyl alcohol, tert-butyl methyl ether, or water), by cooling, or by any combination of such methods.

In some embodiments, a method of preparing Compound 2 comprises a step of isolating Compound 2. It will be appreciated that Compound 2 may be isolated by any suitable means. In some embodiments, Compound 2 (e.g., precipitated or crystallized Compound 2) is separated from a supernatant by filtration. In some embodiments, Compound 2 (e.g., precipitated or crystallized Compound 2) is separated from a supernatant by decanting the supernatant.

In some embodiments, isolated Compound 2 is dried (e.g., in air or under reduced pressure, optionally at elevated temperature, e.g., at about 45° C.).

In some embodiments, a solid form of Compound 2 is prepared by converting one solid form of Compound 2 into another solid form of Compound 2.

### Compositions

The present disclosure also provides compositions comprising one or more solid and/or salt forms of Compound 1. In some embodiments, provided compositions comprise Compound 1 (i.e., Compound 1 Free Base), e.g., Amorphous Compound 1 Free Base, Compound 1 Free Base Form A or Compound 1 Free Base Form B, or a mixture thereof. In some embodiments, provided compositions comprise Compound 2, e.g., Amorphous Compound 1 Hydrochloride, Compound 1 Hydrochloride Form A, Compound 1 Hydrochloride Form B, Compound 1 Hydrochloride Form C, Compound 1 Hydrochloride Form D, Compound 1 Hydrochloride Form E, Compound 1 Hydrochloride Form F, Compound 1 Hydrochloride Form G, Compound 1 Hydrochloride Form H, Compound 1 Hydrochloride Form I, Compound 1 Maleate Form A, Compound 1 Mesylate Form A, or Compound 1 Tosylate Form A, or a mixture thereof.

In some embodiments, a provided composition comprising a crystalline solid form (e.g., a crystalline solid form of Compound 1 or Compound 2) is substantially free of impurities. As used herein, the term “substantially free of impurities” means that the composition contains no significant amount of extraneous matter. Such extraneous matter may include starting materials, residual solvents, or any other impurities that may result from the preparation of and/or isolation of a crystalline solid form. In some embodiments, the composition comprises at least about 90% by weight of a crystalline solid form. In some embodiments, the composition comprises at least about 95% by weight of a crystalline solid form. In some embodiments, the composition comprises at least about 97%, about 98%, or about 99% by weight of a crystalline solid form.

In some embodiments, a composition comprises a crystalline solid form (e.g., a crystalline solid form of Compound 1 or Compound 2) and an amorphous solid form (e.g., Amorphous Compound 1 or Amorphous Compound 2). In some embodiments, a composition comprising a crystalline solid form is substantially free of an amorphous solid form. As used herein, the term “substantially free of an amorphous solid form” means that the composition contains no significant amount of an amorphous solid form. In some embodiments, the composition comprises at least about 90% by weight of a crystalline solid form (e.g., a crystalline solid form of Compound 1 or Compound 2). In some embodiments, the composition comprises at least about 95% by weight of a crystalline solid form. In some embodiments, the composition comprises at least about 97%, about 98%, or about 99% by weight of a crystalline solid form. In some embodiments, the composition comprises no more than about 10% by weight of an amorphous solid form (e.g., an amorphous solid form of Compound 1 or Compound 2). In some embodiments, the composition comprises no more than about 5% by weight of an amorphous solid form. In some embodiments, the composition comprises no more than about 3%, about 2%, or about 1% by weight of an amorphous solid form.

In some embodiments, a composition comprises a free base form (e.g., Compound 1, i.e., Compound 1 Free Base) and a salt form (e.g., Compound 2). In some such embodiments, a free base form is crystalline, amorphous, or a mixture thereof; in some such embodiments, a salt form is crystalline, amorphous, or a mixture thereof.

In some embodiments, a provided composition comprises Compound 1 Hydrochloride and excess hydrochloric acid (HCl). In some embodiments, a composition comprises Compound 1 Hydrochloride and about 15% excess HCl. In some such embodiments, a composition comprises Compound 1 Hydrochloride Form A and about 15% excess HCl.

In some embodiments, a composition comprises a mixture of an amorphous form of Compound 1 and a crystalline form of Compound 1 (i.e., Compound 1 Form A or Compound 1 Form B). In some embodiments, a composition comprises a mixture of an amorphous form of Compound 1 and Compound 1 Form A. In some embodiments, a composition comprises a mixture of an amorphous form of Compound 1 and Compound 1 Form B. In some embodiments, a composition comprises a mixture of an amorphous form of Compound 1, Compound 1 Form A, and Compound 1 Form B.

In some embodiments, a composition comprises a mixture of an amorphous form of Compound 1 and a salt form of Compound 1 (i.e., an amorphous form of Compound 2). In some embodiments, a composition comprises a mixture of an amorphous form of Compound 1 and an amorphous form of Compound 1 Hydrochloride. In some embodiments, a composition comprises a mixture of an amorphous form of Compound 1 and an amorphous form of Compound 1 Tosylate. In some embodiments, a composition comprises a mixture of an amorphous form of Compound 1 and an amorphous form of Compound 1 Mesylate. In some embodiments, a composition comprises a mixture of an amorphous form of Compound 1 and an amorphous form of Compound 1 Maleate.

In some embodiments, a composition comprises a mixture of an amorphous form of Compound 1 and a crystalline salt form of Compound 1 (i.e., a crystalline form of Compound 2). In some embodiments, a composition comprises a mixture of an amorphous form of Compound 1 and a crystalline form of Compound 1 Hydrochloride (e.g., Form A, Form B, Form C, Form D, Form E, Form F, Form G, Form H, or Form I).

In some embodiments, a composition comprises a mixture of an amorphous form of Compound 1 and a crystalline form of Compound 1 Tosylate (e.g., Form A). In some embodiments, a composition comprises a mixture of an amorphous form of Compound 1 and a crystalline form of Compound 1 Mesylate (e.g., Form A). In some embodiments, a composition comprises a mixture of an amorphous form of Compound 1 and a crystalline form of Compound 1 Maleate (e.g., Form A).

In some embodiments, a composition comprises a mixture of an amorphous form of Compound 2 and a crystalline form of Compound 2. In some embodiments, a composition comprises a mixture of an amorphous form of Compound 1 Hydrochloride and Compound 1 Hydrochloride Form A. In some embodiments, a composition comprises a mixture of an amorphous form of Compound 1 Hydrochloride and Compound 1 Hydrochloride Form A Type 1 Lot. In some embodiments, a composition comprises a mixture of an amorphous form of Compound 1 Hydrochloride and Compound 1 Hydrochloride Form A Type 2 Lot. In some embodiments, a composition comprises a mixture of an amorphous form of Compound 1 Hydrochloride and Compound 1 Hydrochloride Form B. In some embodiments, a composition comprises a mixture of an amorphous form of Compound 1 Hydrochloride and Compound 1 Hydrochloride Form C. In some embodiments, a composition comprises a mixture of an amorphous form of Compound 1 Hydrochloride and Compound 1 Hydrochloride Form D. In some embodiments, a composition comprises a mixture of an amorphous form of Compound 1 Hydrochloride and Compound 1 Hydrochloride Form E. In some embodiments, a composition comprises a mixture of an amorphous form of Compound 1 Hydrochloride and Compound 1 Hydrochloride Form F. In some embodiments, a composition comprises a mixture of an amorphous form of Compound 1 Hydrochloride and Compound 1 Hydrochloride Form G. In some embodiments, a composition comprises a mixture of an amorphous form of Compound 1 Hydrochloride and Compound 1 Hydrochloride Form H. In some embodiments, a composition comprises a mixture of an amorphous form of Compound 1 Hydrochloride and Compound 1 Hydrochloride Form I.

In some embodiments, a composition comprises a mixture of crystalline solid forms (e.g., a mixture of one or more of crystalline forms of Compound 1 or Compound 2).

In some embodiments, a composition comprises a mixture of crystalline forms of Compound 1. In some embodiments, a composition comprises a mixture of Compound 1 Form A and Compound 1 Form B.

In some embodiments, a composition comprises a mixture of a crystalline form of Compound 1 and a crystalline form of Compound 2. In some embodiments, a composition comprises a mixture of Compound 1 Form A and a crystalline form of Compound 1 Hydrochloride (e.g., Form A, Form B, Form C, Form D, Form E, Form F, Form G, Form H, or Form I). In some embodiments, a composition comprises a mixture of Compound 1 Form A and Compound 1 Maleate Form A. In some embodiments, a composition comprises a mixture of Compound 1 Form A and Compound 1 Mesylate Form A. In some embodiments, a composition comprises a mixture of Compound 1 Form A and Compound 1 Tosylate Form A.

In some embodiments, a composition comprises a mixture of Compound 1 Form B and a crystalline form of Compound 1 Hydrochloride (e.g., Form A, Form B, Form C, Form D, Form E, Form F, Form G, Form H, or Form I). In some embodiments, a composition comprises a mixture of Compound 1 Form B and Compound 1 Maleate Form A. In some embodiments, a composition comprises a mixture of Compound 1 Form B and Compound 1 Mesylate Form A. In some embodiments, a composition comprises a mixture of Compound 1 Form B and Compound 1 Tosylate Form A.

In some embodiments, a composition comprises two or more crystalline forms of Compound 1 Hydrochloride. In some embodiments, a composition comprises a mixture of Compound 1 Hydrochloride Form A Type 1 Lot and Compound 1 Hydrochloride Form A Type 2 Lot.

In some embodiments, a composition comprises a mixture of Compound 1 Hydrochloride Form A and Compound 1 Hydrochloride Form C. In some such embodiments, a composition comprises a mixture of Compound 1 Hydrochloride Form A Type 1 Lot and Compound 1 Hydrochloride Form C. In some embodiments, a composition comprises a mixture of Compound 1 Hydrochloride Form A Type 2 Lot and Compound 1 Hydrochloride Form C.

In some embodiments, a composition comprises a mixture of Compound 1 Hydrochloride Form A and Compound 1 Hydrochloride Form F. In some such embodiments, a composition comprises a mixture of Compound 1 Hydrochloride Form A Type 1 Lot and Compound 1 Hydrochloride Form F. In some embodiments, a composition comprises a mixture of Compound 1 Hydrochloride Form A Type 2 Lot and Compound 1 Hydrochloride Form F.

In some embodiments, a composition comprises a mixture of Compound 1 Hydrochloride Form B and Compound 1 Hydrochloride Form F. In some embodiments, a composition comprises a mixture of Compound 1 Hydrochloride Form B and Compound 1 Hydrochloride Form H. In some embodiments, a composition comprises a mixture of Compound 1 Hydrochloride Form F and Compound 1 Hydrochloride Form H.

### PHARMACEUTICAL COMPOSITIONS

In some embodiments, the present disclosure provides a pharmaceutical composition comprising a solid form of Compound 1 or Compound 2. In some embodiments, a pharmaceutical composition is a capsule comprising a solid form of Compound 1 or Compound 2. In some such embodiments, the capsule is a gelatin capsule.

In some embodiments, a pharmaceutical composition is a capsule comprising Compound 1 Hydrochloride (e.g., with no excipients). In some embodiments, a pharmaceutical composition is a capsule comprising Compound 1 Hydrochloride Form A (e.g., with no excipients). In some embodiments, a pharmaceutical composition is a capsule comprising Compound 1 Hydrochloride Form A (e.g., with no excipients), wherein Form A is a channel hydrate comprising from about 8% to about 13% water.

In some embodiments, provided pharmaceutical compositions comprise 10 mg, 50 mg, or 250 mg Compound 1. In some embodiments, provided pharmaceutical compositions comprise 100 mg or 200 mg Compound 1. It will be appreciated that Compound 1 may be provided and/or utilized in a pharmaceutical composition as, e.g., a salt form of Compound 1. Accordingly, reference to an amount of Compound 1 in a pharmaceutical composition means the amount of Compound 1 in a free base form. For example, “50 mg Compound 1” means, e.g., approx. 53.4 mg of Compound 1 Hydrochloride anhydrate, approx. 58.4 mg of Compound 1 Hydrochloride trihydrate, and approx. 58.9 mg of Compound 1 Mesylate anhydrate, etc.

In some embodiments, provided pharmaceutical compositions comprise 10 mg, 50 mg, or 250 mg Compound 1 as a Compound 1 Hydrochloride Form A solid form. In some embodiments, provided pharmaceutical compositions comprise 100 mg or 200 mg Compound 1 as a Compound 1 Hydrochloride Form A solid form.

In some embodiments, provided pharmaceutical compositions are a capsule comprising 10 mg Compound 1 (e.g., as a Compound 1 Hydrochloride Form A solid form). In some embodiments, provided pharmaceutical compositions are a capsule comprising 50 mg Compound 1 (e.g., as a Compound 1 Hydrochloride Form A solid form). In some embodiments, provided pharmaceutical compositions are a capsule comprising 100 mg Compound 1 (e.g., as a Compound 1 Hydrochloride Form A solid form). In some embodiments, provided pharmaceutical compositions are a capsule comprising 200 mg Compound 1 (e.g., as a Compound 1 Hydrochloride Form A solid form). In some embodiments, provided pharmaceutical compositions are a capsule comprising 250 mg Compound 1 (e.g., as a Compound 1 Hydrochloride Form A solid form).

In some embodiments, a pharmaceutical composition comprises a mixture described herein (e.g., a mixture of one or more solid forms described herein).

In some embodiments, a provided composition is a pharmaceutical composition comprising a solid form of Compound 1 or Compound 2 and a pharmaceutically acceptable carrier. In some embodiments, a pharmaceutically acceptable carrier comprises one or more fillers, disintegrants, lubricants, and/or diluents such as pharmaceutically acceptable starches (e.g. corn, potato or tapioca starch), sugars, artificial sweetening agents, powdered celluloses, such as crystalline and microcrystalline celluloses, flours, gelatins, gums, etc. as discussed in greater detail below.

Pharmaceutical compositions described herein can be administered to a subject by any known method, such as orally, parenterally, perineurally, transmucosally, transdermally, intramuscularly, intravenously, intradermally, subcutaneously, intraperitoneally, intraventricularly, intracranially or intratumorally. In some embodiments, a pharmaceutical composition described herein is an oral formulation. Oral formulations comprising one or more solid forms of Compound 1 or Compound 2 can be in any conventionally used oral form, including tablets, capsules, buccal forms, troches, or lozenges. In some embodiments, a provided pharmaceutical composition is a capsule formulation. In some embodiments, a provided pharmaceutical composition is a tablet formulation.

Tablet formulations can be made by conventional compression, wet granulation, or dry granulation methods and utilize pharmaceutically acceptable diluents (fillers), binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar.

Pharmaceutically acceptable fillers can be any filler known in the art including, but not limited to, lactose, microcrystalline cellulose, sucrose, mannitol, calcium phosphate, calcium carbonate, powdered cellulose, maltodextrin, sorbitol, starch, or xylitol.

Pharmaceutically acceptable disintegrants suitable for use in provided formulations can be selected from those known in the art, including pregelatinized starch, cornstarch, potato starch, and sodium starch glycolate. Other useful disintegrants include croscarmellose sodium, crospovidone, starch, alginic acid, guar gum, silicon dioxide, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). Disintegrant(s) useful herein can make up from about 4% to about 40% of the composition by weight, preferably from about 15% to about 35%, more preferably from about 20% to about 35%.

Pharmaceutically acceptable diluents are well known to those skilled in the art. For example, diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable binders include, for example, acacia, cornstarch, gelatin, carbomer, ethyl cellulose, guar gum, hydroxypropyl cellulose, hydroxypropyl methyl cellulose, and povidone.

Pharmaceutical compositions may also comprise buffers, for example, e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Provided pharmaceutical formulations can also contain an antioxidant or a mixture of antioxidants, such as ascorbic acid. Other antioxidants which can be used include sodium ascorbate and ascorbyl palmitate, preferably in conjunction with an amount of ascorbic acid. An example range for the antioxidant(s) is from about 0.5% to about 15% by weight, most preferably from about 0.5% to about 5% by weight.

Pharmaceutical formulations described herein can be used in an uncoated or non-encapsulated solid form. In some embodiments, pharmacological compositions are optionally coated with a film coating, for example, comprising from about 0.3% to about 8% by weight of the overall composition. Film coatings useful with provided formulations are known in the art and generally consist of a polymer (usually a cellulosic type of polymer), a colorant and a plasticizer. Additional ingredients such as wetting agents, sugars, flavors, oils and lubricants may be included in film coating formulations to impart certain characteristics to the film coat. Compositions and formulations provided herein may also be combined and processed as a solid, then placed in a capsule form, such as a gelatin capsule.

### Uses of Provided Compounds and Compositions Thereof

Provided herein are methods of treating a subject comprising administering a solid form of Compound 1 or Compound 2 (e.g., by administering a composition that comprises and/or delivers the solid form of Compound 1 or Compound 2) to the subject in need thereof. In some embodiments, provided methods comprise administering a solid form of Compound 1 to a subject in need thereof. In some embodiments, provided methods comprise administering a solid form of Compound 2 to a subject in need thereof. In some embodiments, provided methods comprise administering Compound 1 Hydrochloride (e.g., Form A) to a subject in need thereof. In some such embodiments, a solid form of Compound 1 or Compound 2 is administered as a pharmaceutical composition (e.g., as a capsule composition).

In some embodiments, the present disclosure provides methods of treating diseases, disorders, and conditions (e.g., according to methods provided herein). In some embodiments, provided methods are useful for reducing fibrosis in a subject in need thereof. In some embodiments, provided methods are useful for treating a disease, disorder, or condition characterized by or otherwise associated with fibrosis. The present disclosure encompasses the recognition that treating fibrosis (e.g., using provided methods) instead of the underlying etiology may allow for broadly applicable antifibrotic therapies. It will be appreciated that provided methods may be suitable for reducing fibrosis in a variety of tissues and/or organs; the present disclosure contemplates use of a solid form of Compound 1 or Compound 2 for treating diseases, disorders, and conditions characterized by or otherwise associated with fibrosis in any suitable tissue and/or organ. For example, in some embodiments, provided methods are suitable for treating diseases, disorders and conditions that are or comprise fibrosis of gastrointestinal tract, heart, kidney, lung, liver, muscle, pancreas, and/or skin. It will be appreciated that provided methods may be suitable for treating diseases, disorders, and conditions in which fibrosis is the sole or a predominant component, as well as those in which fibrosis is a secondary component (e.g., a symptom and/or result of an underlying disease, disorder, or condition). It will also be appreciated that there are a variety of sources or causes of fibrosis. Adults with polycystic kidney disease often also develop asymptomatic cysts in the liver, pancreas, ovaries and spermatic duct, in addition to cysts in the kidney. In some embodiments, provided methods are suitable for treating diseases, disorders, and conditions characterized by or otherwise associated with cysts (e.g., in the kidney, liver, pancreas, ovaries, spermatic duct, etc.).

In some embodiments, certain injuries can progress to development of fibrosis. In some embodiments, provided methods are useful for treating acute injuries (e.g., acute organ injuries, such as acute lung injury, acute liver injury, or acute kidney injury), as well as for treating chronic injuries (e.g., chronic organ injuries, such as chronic lung injury, chronic liver injury, or chronic kidney injury). In some embodiments, provided methods are useful for treating fibrosis associated with an acute injury, such as that incurred from trauma and/or surgery and/or infection (e.g., a viral infection). For example, in some embodiments, provided methods are useful for acceleration of wound healing, reduction of post-surgical scarring, and/or reduction of adhesion formation. In some embodiments, provided methods are useful for treating damaged and/or ischemic organs, transplants, or grafts, as well as ischemia/reperfusion injury or post-surgical scarring. For example, in some embodiments, provided methods are useful for promoting vascularization of a damaged and/or ischemic organ, transplant, or graft, ameliorating ischemia/reperfusion injury (e.g., in brain, heart, liver, or kidney), normalizing myocardial perfusion resulting from chronic cardiac ischemia or myocardial infarction, and/or developing or augmenting collateral vessel development after vascular occlusion or to ischemic tissues or organs.

### Pulmonary Diseases, Disorders, and Conditions

In some embodiments, provided methods are useful for treating pulmonary diseases, disorders, and conditions. In some embodiments, provided methods are useful for treating pulmonary fibrosis. In some embodiments, provided methods are useful for treating pulmonary fibrosis secondary to, or otherwise associated with, an underlying indication. In some embodiments, provided methods are useful for treating interstitial lung diseases (e.g., fibrosing interstitial lung diseases). In some embodiments, provided methods are useful for treating pneumonias (e.g., idiopathic interstitial pneumonias). In some embodiments, provided methods are useful for treating idiopathic pulmonary fibrosis (IPF). In some embodiments, provided methods are useful for treating pulmonary fibrosis associated with an infection (e.g., a bacterial, viral, or fungal infection). In some embodiments, provided methods are useful for treating pulmonary fibrosis associated with a viral infection (e.g., an influenza or coronavirus infection, such as COVID-19).

In some embodiments, a fibrotic disease to be treated by methods of the present disclosure is pulmonary fibrosis. Pulmonary fibrosis is a chronic, progressive, and ultimately fatal interstitial lung disease resulting from epithelial cell injury due to many factors. Upon epithelial injury, activation of inflammatory cells and fibroblasts/myofibroblasts involves a cascade of cytokines/chemokines, growth factor network and deposit extracellular matrix, including collagen), which leads to pulmonary fibrosis and respiratory failure. Pulmonary fibrosis causes high morbidity and mortality. At least five million people worldwide and 200,000 people in the United States suffer from pulmonary fibrosis. There is an unmet critical need for effective and affordable treatments for acute and chronic lung injuries.

Numerous endogenous and exogenous factors can provide primary stimuli for pulmonary fibrosis. Dust, silica, smoke, aerosolized toxins, infections and certain medicines have the potential to injure the lung and set the stage for the development of chronic pulmonary fibrosis. For example, viral infections may cause lung damage and/or otherwise develop into pulmonary fibrosis. In a study of patients diagnosed with H1N1 infection, it was found that 10% of patients developed post-ARDS fibrosis (Mineo, G., et al. Radiol. Med. 2012; 117:185-200). In COVID-19 patients, acute respiratory distress syndrome (ARDS) developed in 17-29% of hospitalized patients (Huang C, e al. The Lancet. 2020 Jan. 24).

Pulmonary fibrosis is associated with pronounced morbidity with high impact on economic burden. A clinical study indicated that the total direct costs for patients with pulmonary fibrosis were $26,378 per person and other study indicated that the mean Medicare costs for a lung transplant recipient was $131,352. The prevalence of pneumoconiosis (a disease caused by inhalation of dust and silica that causes inflammation and lung fibrosis) caused direct and indirect economic losses of around 28 billion yuan in China (4.3 billion US dollars) for 1 year.

In some embodiments, a fibrotic disease to be treated by methods of the present disclosure is idiopathic pulmonary fibrosis. Idiopathic pulmonary fibrosis (IPF) is a chronic, irreversible, and progressive fibrotic disorder of the lower respiratory tract that typically affects adults over the age of 40. Idiopathic interstitial pneumonias (IIP) are diffuse parenchymal lung diseases, of which IPF is the most common and severe type of fibrotic lung disease. It is anatomically characterized by scarring of the lungs with a pattern of usual interstitial pneumonia (UIP) on high resolution computed tomography or histologic appearance on lung biopsy. Clinically IPF follows a relentless course of progressive, irreversible, and debilitating disease characterized by exertional dyspnea and cough. Median survival following diagnosis of IPF ranges between 2 and 5 years, lower than that for many common cancers (Ley, B., et al. Am. J. Respir. Crit. Care Med. 2011; 183:431-440; Seigel, R. L., et al. C A. Cancer J. Clin. 2016; 66:7-30). Fibrotic process in IPF is progressive and, regardless of the nature of the initial injury, may follow a common pathway characterized by alveolar epithelial cell (AEC) dysfunction. The injury to epithelial cells and basement membrane results in complex cell and cytokine interactions that extend the fibrotic process to the alveolar walls, alveolar lumen, and then adjacent areas of lung parenchyma. Both epithelial and basement membrane injury appear necessary for the development of intraluminal fibrosis (Crapo, J. D., et al. Am. Rev. Respir. Dis. 1982; 126:332-7).

Normal alveolar epithelium is comprised predominantly of type I epithelial cells (AECs1), with a relatively small number of type II epithelial cells (AECs2). After injury, AECs2 proliferate and differentiate into AECs1 and are normally responsible for re-epithelialization of injured alveoli. This is accomplished by several processes involving coagulation cascade, angiogenesis, fibroblast activation and migration, and collagen synthesis and proper alignment (Betensley, A., et al. J. Clin. Med. 2016; 6:2). Many chemokines such as transforming growth factor beta 1 (TGF-β1), platelet-derived growth factor (PDGF), vascular endothelial growth factor (VEGF), and fibroblast growth factor (FGF) play a key role in these processes. Regardless of the initial injury, the progression of fibrosis in IPF follows a common and complex path in which the AECs, fibroblasts, and endothelial cells produce an array of cytokines and growth factors that stimulate fibroblast proliferation and matrix synthesis.

### Hepatic Diseases, Disorders, and Conditions

In some embodiments, provided methods are useful for treating hepatic diseases, disorders, and conditions. In some embodiments, provided methods are useful for treating hepatic fibrosis (e.g., fibrotic liver disease). In some embodiments, provided methods are useful for treating cirrhosis. In some embodiments, provided methods are useful for treating hepatic fibrosis and/or cirrhosis secondary to, or otherwise associated with, an underlying indication. In some embodiments provided methods are useful for treating hepatic fibrosis associated with hepatitis C, hepatitis B, delta hepatitis, chronic alcoholism, nonalcoholic steatohepatitis (NASH), extrahepatic obstructions (e.g., stones in bile duct), cholangiopathies (e.g., primary biliary cirrhosis or sclerosing cholangitis), autoimmune liver disease, or inherited metabolic disorders (e.g., Wilson's disease, hemochromatosis, or alpha-1 antitrypsin deficiency).

In some embodiments, a fibrotic disease to be treated by methods of the present disclosure is liver fibrosis. Liver fibrosis is a scarring response of the liver to chronic liver injury; when fibrosis progresses to cirrhosis, morbid complications can develop. In fact, end-stage liver fibrosis or cirrhosis is the seventh leading cause of death in the United States, and afflicts hundreds of millions of people worldwide; deaths from end-stage liver disease in the United States are expected to increase, mainly due to the hepatitis C epidemic. In addition to the hepatitis C virus, many other forms of chronic liver injury also lead to end-stage liver disease and cirrhosis, including other viruses such as hepatitis B and delta hepatitis, chronic alcoholism, non-alcoholic steatohepatitis, extrahepatic obstructions (e.g., stones in the bile duct), cholangiopathies (e.g., primary biliary cirrhosis and sclerosing cholangitis), autoimmune liver disease, and inherited metabolic disorders (e.g., Wilson's disease, hemochromatosis, and alpha-I anti trypsin deficiency).

Treatment of liver fibrosis has traditionally focused on eliminating a primary injury. For extrahepatic obstructions, biliary decompression is the recommended mode of treatment whereas patients with Wilson's disease are treated with zinc acetate. Treatments for other chronic liver diseases such as hepatitis B, autoimmune hepatitis and Wilson's disease are also associated with many side effects, while primary biliary cirrhosis, primary sclerosing cholangitis and non-alcoholic fatty liver disease have no effective treatment other than liver transplantation.

While transplantation may currently be the most effective cure for liver fibrosis, mounting evidence indicates that not only fibrosis, but even cirrhosis is reversible. Unfortunately, patients often present with advanced stages of fibrosis and cirrhosis, when many therapies such as antivirals can no longer be safely used due to their side effect profile. Such patients would benefit enormously from effective antifibrotic therapy, because attenuating or reversing fibrosis may prevent many late stage complications such as infection, ascites, and loss of liver function and preclude the need for liver transplantation.

### Renal Diseases, Disorders, and Conditions

In some embodiments, provided methods are useful for treating renal diseases, disorders, and conditions. In some embodiments, provided methods are useful for treating renal fibrosis. In some embodiments, provided methods are useful for treating renal fibrosis secondary to, or otherwise associated with, an underlying indication. In some embodiments, provided methods are useful for treating renal fibrosis associated with renal failure, renal obstruction, renal trauma, renal transplantation, chronic kidney disease, diabetes, hypertension, radiocontrast nephropathy, immune-mediated glomerulonephritides (e.g., lupus nephritis, ANCA-associated glomerulonephritides (e.g., Wegener's granulomatosis, microscopic polyangiitis, or renal limited vasculitis), anti-GBM nephropathy, IgA nephropathy, membranous glomerulonephritis, or focal and segmental glomerulosclerosis), non-immune-mediated glomerulonephritides (e.g., polycystic kidney disease, collagen type III glomerulopathy, nail-patella syndrome, or Alport syndrome), minimal change disease, or nephrotic syndrome (e.g., steroid-resistant nephrotic syndrome). In some embodiments, provided methods are useful for treating nephrotic syndrome and/or diseases, disorders, or conditions associated with nephrotic syndrome (e.g., focal and segmental glomerulosclerosis, minimal change disease, and membranous nephropathy). In some embodiments, provided methods are useful for treating a fibrotic disease of the kidney that is or comprises: focal segmental glomerulosclerosis (FSGS), steroid resistant nephrotic syndrome (SRNS), proteinuria, lupus nephritis, minimal change disease, an anti-neutrophil cytoplasmic antibody (ANCA)-associated glomerulonephritis, Alport syndrome, anti-globular basement membrane (anti-GBM) nephropathy, IgA nephropathy, membranous glomerulonephritis (MG), autosomal dominant polycystic kidney disease (ADPKD), collagen type III glomerulopathy, nail-patella syndrome, or chronic kidney disease. In some embodiments, provided methods are useful for treating a fibrotic disease of the kidney that is or comprises an anti-neutrophil cytoplasmic antibody (ANCA)-associated glomerulonephritis. In some embodiments, ANCA-associated glomerulonephritis is selected from Wegener's granulomatosis, microscopic polyangiitis (MPA), or renal limited vasculitis. In some embodiments, provided methods are useful for treating focal and segmental glomerulosclerosis (FSGS). In some embodiments, provided methods are useful for treating Alport syndrome. In some embodiments, provided methods are useful for treating polycystic kidney disease (e.g., autosomal dominant polycystic kidney disease or autosomal recessive polycystic kidney disease).

In some embodiments, provided methods are useful for treating primary proteinuric kidney disease (PPKD). In some embodiments, provided methods are useful for treating primary glomerular diseases (PGDs). PGDs are among the leading causes of chronic kidney disease and end-stage kidney disease in the world. PGDs predominantly affect younger patients, significantly reducing their quality of life, productivity, and longevity. FSGS, membranous nephropathy (MN), and IgA nephropathy are among the three most common primary glomerular diseases in adults. Accordingly, in some embodiments, provided methods are useful for treating FSGS. In some embodiments, provided methods are useful for treating MN. In some embodiments, provided methods are useful for treating IgA nephropathy.

Currently, there are no therapies approved specifically for PPKDs and/or PGDs. Various immunosuppressive and/or cytotoxic agents primarily approved for other indications are first-line therapy for patients considered to be at a high risk of progression. Such therapies aim to induce remission, defined as a normalization of the urinary protein excretion. The patients who undergo a complete or a partial remission have significantly improved renal prognosis. Unfortunately, remission rates with immunosuppressive agents and cytotoxic therapy range only between 30-60% depending on the underlying disease, and up to 50% of these patients subsequently experience a relapse. Relapses in proteinuria are frequently treated with repeat administration of immunosuppressive and/or cytotoxic therapy; however, long-term treatment with these agents is limited by their significant dose-limiting toxicity. Some of the drugs, such as calcineurin inhibitors (CNIs), have a narrow therapeutic index necessitating close monitoring of the drug levels. Long-term use of CNIs is associated with hypertension, nephrotoxicity, and metabolic abnormalities such as diabetes and dyslipidemia. In most patients, cessation of calcineurin inhibitors results in the relapse of proteinuria (Meyrier, A. et al., Kidney International. 1994; 45(5):1446-56). A significant number of patients eventually become either resistant or dependent on these toxic agents. Some of these glomerular diseases also recur after renal transplantation posing unique management problems (Choy, B. Y., et al., Am. J. Transplant. 2006; 6(11):2535-42).

In some embodiments, provided methods are useful for treating patients with proteinuria (e.g., persistent proteinuria). It is well established that higher rates of urinary protein excretion are associated with worse prognosis, and therapies that reduce proteinuria are desirable for improving renal outcomes. Patients with persistent proteinuria (e.g., who continue to have >1 gram of proteinuria per day) are at high risk of progressing to end-stage kidney disease (ESKD). Risk of progression is significantly increased in presence of a reduced estimated glomerular filtration rate (eGFR) at the time of diagnosis or during the course of the disease. Patients with persistent proteinuria also develop further complications of chronic kidney disease (CKD) such as dyslipidemia, cardiovascular disease, abnormalities in mineral-bone metabolism, and hypertension, resulting in significant increases in morbidity and mortality and utilization of health care resources.

In addition to immunosuppressive agents, standard of care for patients with persistent proteinuria includes treatment with the renin-angiotensin-aldosterone system (RAAS) blockers, most commonly ACE inhibitors or angiotensin-receptor blockers (ARBs). The RAAS blockers reduce proteinuria and improve clinical outcomes in proteinuric renal diseases regardless of the etiology. Other standard of care recommendations include aggressive blood pressure control (<130/80), and HMG-CoA reductase inhibitors (e.g., statins) in patients with hyperlipidemia. The inhibitors of the mineralocorticoid receptor and sodium glucose co-transporter-2 (SGLT-2) are increasingly being used in these patients as well.

In some embodiments, provided methods are useful for treating primary glomerular diseases (e.g., FSGS, membranous nephropathy, or IgA nephropathy) and persistent proteinuria.

Several growth factor receptors have been implicated in the development of fibrosis of the kidney (Liu, F., et al. Int. J. Mol. Sci. 2016 Jun. 20; 17(5), PMCID:PMC4926504). Platelet-derived growth factor receptor beta (PDGFRβ) is postulated to play a particularly important role in the development of renal fibrosis (Floege, J., et al. J. Am. Soc. Nephrol. 2008 January; 19(1):12-23; Ostendorf, T., et al. Pediartr. Nephrol. 2012 July; 27(7):1041-50; Ostendorf. T., et al. Kidney Int. Suppl. (2011) 2014 November; 4(1):65-9, PMCID:PMC4536969; Abbound, H. E. Annu. Rev. PHysiol. 1995; 57:297-309).

In some embodiments, a kidney disease to be treated by methods of the present disclosure is nephrotic syndrome (NS). NS is a group of rare renal diseases, including focal and segmental glomerulosclerosis (FSGS), minimal change disease (MCD), and membranous nephropathy. FSGS is a rare disease that attacks the kidney's filtering units (glomeruli) causing serious scarring which leads to permanent kidney damage and even failure (Fogo, A. B. Nat. Rev. Nephrol. 2015 February; 11(2):76-87, PMCID:PMC4772430). It will be appreciated that there are at least three types of FSGS. Primary FSGS is FSGS that has no known cause (also referred to as idiopathic FSGS). Secondary FSGS is caused by one or more factors such as infection, drug toxicity, diseases such as diabetes or sickle cell disease, obesity, or other kidney diseases. Genetic FSGS (also called familial FSGS) is caused by one or more genetic mutations. Primary FSGS is idiopathic in nature. Manifestations of this disease include hypoalbuminemia and edema, lipid abnormalities and nephrotic range proteinuria. More than 5400 patients are diagnosed with FSGS every year (O'Shaughnessy, M. M., et al. Nephrol. Dial. Transplant 2018 Apr. 1; 33(4):661-9). However, this is considered an underestimate because a limited number of biopsies are performed, and the number of FSGS cases is rising more than any other cause of NS. Standard of care for this patient population is steroid therapy. Current treatments for FSGS include corticosteroids, calcineurin inhibitors, mycophenolate mofetil, adrenocorticotropic hormone (ATCH), and rituximab; these are effective in at most 25-40% of patients. A subset of this population is resistant to steroids (steroid-resistant, or SR), and proteinuria, which is toxic to renal tubules, remains uncorrected. Consequently, this subset proceeds relatively rapidly to end-stage renal disease (ESRD). There is therefore an urgent need to develop therapies that reduce proteinuria in primary SR-FSGS (Nourbakhsh, N. and Mak, R. H. Pediatric Health Med. Ther. 2017; 8:29-37, PMCID:PMC5774596).

In some embodiments, a kidney disease to be treated by methods of the present disclosure is minimal change disease (MCD). MCD is a kidney disease in which large amounts of protein are lost in the urine. It is one of the most common causes of the nephrotic syndrome worldwide. In children, MCD is usually primary (or idiopathic), but in adults, the disease is usually secondary. Secondary causes for MCD include allergic reactions, use of certain painkillers such as non-steroidal anti-inflammatory drugs (NSAIDs), tumors, or viral infections.

In some embodiments, a kidney disease to be treated by methods of the present disclosure is membranous glomerulonephritis (MG or MGN), also known as membranous nephropathy (MN). MG or MGN is a slowly progressive renal disease caused by immune complex formation in the glomerulus. Immune complexes are formed by binding of antibodies to antigens in the glomerular basement membrane. The antigens may be part of the basement membrane, or deposited from elsewhere by the systemic circulation.

In some embodiments, a kidney disease to be treated by methods of the present disclosure is anti-neutrophil cytoplasmic antibody (ANCA)-associated glomerulonephritis. Anti-neutrophil cytoplasmic antibody (ANCA)-associated glomerulonephritis is a rapidly progressive renal disease and includes, e.g., Wegener's granulomatosis, microscopic polyangiitis, and renal limited vasculitis. Wegener's granulomatosis is an organ- and/or life-threatening autoimmune disease of unknown etiology. The classical clinical triad consists of necrotizing granulomatous inflammation of the upper and/or lower respiratory tract, necrotizing glomerulonephritis, and an autoimmune necrotizing systemic vasculitis affecting predominantly small vessels. The detection of anti-neutrophil cytoplasmic antibodies directed against proteinase 3 (PR3-ANCA) is a highly specific indicator for Wegener's granulomatosis. Microscopic polyangiitis is a disorder that causes blood vessel inflammation (vasculitis), which can lead to organ damage. The kidneys, lungs, nerves, skin, and joints are the most commonly affected areas of the body. MPA is diagnosed in people of all ages, all ethnicities, and both genders. The cause of this disorder is unknown. Renal limited vasculitis is a type of anti-neutrophil cytoplasmic antibody (ANCA)-associated vasculitis that presents with only a renal manifestation; no other organs, including lungs, are involved.

In some embodiments, a kidney disease to be treated by methods of the present disclosure is lupus nephritis. Lupus nephritis is inflammation of the kidney that is caused by the autoimmune disease systemic lupus erythematous (SLE). With lupus, the body's immune system targets its own body tissues; lupus nephritis occurs when lupus involves the kidneys.

In some embodiments, a kidney disease to be treated by methods of the present disclosure is anti-globular basement membrane (anti-GBM) nephropathy. Anti-globular basement membrane (anti-GBM) nephropathy is a disease that occurs as a result of injury to small blood vessels (capillaries) in the kidneys and/or lungs. In anti-GBM disease, autoantibodies are targeted to the basement membrane in capillary blood vessels of the kidneys and lung, where they target and damage GBM.

In some embodiments, a kidney disease to be treated by methods of the present disclosure is IgA nephropathy, also known as Berger's disease. IgA nephropathy is a kidney disease that occurs when IgA deposits build up in the kidneys, causing inflammation that damages kidney tissues. IgA nephropathy affects the kidneys by attacking the glomeruli. The buildup of IgA deposits inflames and damages the glomeruli, causing the kidneys to leak blood and protein into the urine. The damage may lead to scarring of the nephrons that progresses slowly over many years. Eventually, IgA nephropathy can lead to end-stage kidney disease.

In some embodiments, a kidney disease to be treated by methods of the present disclosure is collagen type III glomerulopathy. Collagen type III glomerulopathy, also known as collagenic or collagenofibrotic glomerulopathy, is characterized by pathological accumulation of collagen type III in glomeruli. Collagen type III glomerulopathy presents either in childhood, often with a family history suggesting autosomal recessive inheritance, or in adults as a sporadic occurrence. Proteinuria is a typical manifestation, with progression to end stage renal disease (ESRD) in approximately 10 years. Although there is markedly elevated serum precursor collagen type III protein in the circulation, the usual manner of diagnosis is with kidney biopsy, which discloses type III collagen in subendothelial aspects of capillary walls and often in the mesangial matrix.

In some embodiments, a kidney disease to be treated by methods of the present disclosure is nail-patella syndrome. Nail-patella syndrome is a multi-organ disorder caused by mutations in the LMX1B gene. Nail-patella syndrome manifests with orthopedic and cutaneous deformities, as well as kidney complications due to development of structural lesions of collagen type III within glomerular basement membranes. Although the structural lesions may be asymptomatic, they are usually accompanied by proteinuria.

In some embodiments, a kidney disease to be treated by methods of the present disclosure is Alport syndrome (AS). AS is a genetic condition characterized by kidney disease, hearing loss, and eye abnormalities. Most affected individuals experience progressive loss of kidney function, usually resulting in end-stage kidney disease. In 80% of cases, Alport syndrome is inherited in an X-linked manner and is caused by mutation(s) in the COL4A5 gene. In other cases, it can be inherited in either an autosomal recessive, or rarely in an autosomal dominant manner, and is caused by mutation(s) in the COL4A3 and/or COL4A4 genes. Current therapies include hearing aid, hemodialysis, peritoneal dialysis and kidney transplantation.

In some embodiments, a kidney disease to be treated by methods of the present disclosure is polycystic kidney disease (e.g., autosomal recessive polycystic kidney disease (ARPKD)—congenital hepatic fibrosis (CHF)). ARPKD-CHF is a highly aggressive fibropolycystic disease that is characterized by the formation and expansion of fluid-filled cysts in the kidneys, enlargement of the kidneys and progressive fibrosis of both the kidney and the liver (Hartung, E. A., and Guay-Woodford, L. M. Pediatrics 2014 September; 134(3):e833-e845; Gunay-Aygun, M., et al. J. Pediatr. 2006 August; 149(2):159-64). Adults with polycystic kidney disease often also develop asymptomatic cysts in the liver, pancreas, ovaries and spermatic duct, in addition to cysts in the kidney. Caroli's disease, which manifests as cystic dilatation of the intrahepatic ducts, often accompanies ARPKD-CHF (Sung, J. M., et al. Clin. Nephrol. 1992 December; 38(6):324-8). In some embodiments, a subject is suffering from, susceptible to, or at risk of Caroli's disease. Afflicted children that survive past two years of age more often than not require renal and/or hepatic transplantation by age ten. The need for transplantation is driven as much by progressive organ dysfunction as by significant enlargement of the diseased organ(s) accompanied by severe pain (www.arpkdchf.org).

In some embodiments, a kidney disease to be treated by methods of the present disclosure is or includes renal cysts. Aberrant signaling by tyrosine kinases, including platelet-derived growth factor (PDGF) and vascular endothelial growth factor (VEGF) and their receptors (R), PDGFR and VEGFR/KDR, respectively, has been implicated in the formation and expansion of renal cysts. A PDGF-driven ciliopathy and/or overexpression of PDGF in the cyst lining and adjacent tubules are thought to, in part, drive renal cystic disease (Torres, V. E., et al. Lancet 2007 Apr. 14; 369(9569):1287-301; Park. J. H. et al. Polycystic Kidney Disease Brisbane; 2015:375-96; Nakamura, T., et al. J. Am. Soc. Nephrol. 1993 January; 3(7):1378-86). Cowley et al. posited that elevated and abnormal c-myc proto-oncogene expression drives ARPKD (Proc. Natl. Acad. Sci. U.S.A. 1987 December: 84(23):8394-8); c-myc expression is controlled by PDGF (Frick, K. K., et al. C. J. Biol. Chem. 1988 Feb. 25; 263(6):2948-52).

VEGF-driven angiogenesis is also thought to contribute to the growth of renal cysts, and inhibition of VEGFR/KDR signaling is associated with decreased tubule cell proliferation, decreased cystogenesis, and blunted renal enlargement (Bello-Ruess, E., et al. Kidney Int. 2001 July; 60(1):37-45; Schrijvers, B. F., et al. Kidney Int. 2004 June; 65(6):2003-17). Nevertheless, the role of VEGF in fibropolycystic disease is more controversial, with at least some reports suggesting that this growth factor might be associated with disease mitigation (Spirli, C., et al. Gastroenterology 2010 January; 138(1):360-71). Aside from their roles in renal cyst formation and expansion, it is being recognized in ARPKD-CHF that aberrant PDGF and VEGF signaling are also associated with extracellular matrix deposition in the liver and kidney (Rajekar, H., et al. J. Clin. Exp. Hepatol. 2011 September; 1(2):94-108; Jiang, L., et al. Biomed. Res. Int. 2016; 2016:4918798; Tao, Y., et al. Kidney Int. 2007 December; 72(11); 1358-66).

### Dermal Diseases, Disorders, and Conditions

In some embodiments, provided methods are useful for treating dermal diseases, disorders, and conditions. In some embodiments, provided methods are useful for treating dermal fibrosis. In some embodiments, provided methods are useful for treating dermal fibrosis secondary to, or otherwise associated with, an underlying indication. In some embodiments, provided methods are useful for treating scleroderma and/or systemic sclerosis (e.g., diffuse systemic sclerosis or limited systemic sclerosis).

In some embodiments, a fibrotic disease to be treated by methods of the present disclosure is scleroderma and/or systemic sclerosis (SSc). Scleroderma, which literally means hard skin, is a chronic fibrotic disorder of unknown etiology that affects the skin and other internal organs (SSc) (www.scleroderma.org). Many patients who suffer from scleroderma/SSc also have loss of lung function. Scleroderma/SSc and related diseases afflict approximately 400,000 to 990,000 people in the USA every year. Mortality and morbidity in scleroderma/SSc are very high and directly related to the extent of fibrosis of the involved organs (Hinchcliff, M. and Varga, J. Am. Fam. Physician 2008 October; 78(8):961-8). A number of international studies suggest that scleroderma/SSc occurs much more frequently in the USA than elsewhere and that it occurs three to four times more frequently among women (Mayes, M. D., et al. Arthritis Rheum. 2003 August; 48(8):2246-55).

According to several studies, the total economic cost of scleroderma/SSc in the USA reaches $1.5 billion annually. Morbidity represents the major cost burden, associated with $820 million (55%) of total cost. The high cost of scleroderma/SSc reflects the burden of chronic disease affecting an early age of disease onset and its high morbidity (Wilson, L. Semin. Arthritis Rheum. 1997 October; 27(2):73-84). Hence, there is a critical need for effective and affordable therapies.

Scleroderma/SSc can be classified in terms of the degree and location of the skin involvement and has been categorized into two major groups—diffuse and limited. The diffuse form of scleroderma/SSc involves symmetric thickening of skin of the extremities, face and trunk. Organs affected include the esophagus, intestines, lungs, heart, and kidneys (Mayes, M. D. Semin. Cutan. Med. Surg. 1998 March; 17(1):22-6; Jacobsen, L. et al. J. Am. Acad. Dermatol. 2003 August; 49(2):323-5). The limited form of scleroderma/SSc tends to be confined to the skin of fingers and face. The limited form of scleroderma/SSc is the CREST variant of scleroderma/SSc based on the clinical pattern of calcinosis with tiny deposits of calcium in the skin, Raynaud's phenomenon in the fingers, toes, nose, tongue, or ears, poor functioning of muscle of esophagus, sclerodactyly of the skin of the fingers or toes, and telangiectasias on the face, hands and mouth (Winterbauer, R. H. Bull. Johns Hopkins Hospital 1964; 114:361-83; Wollheim, F. A. Classification of systemic sclerosis. Visions and reality. Rheumatology (Oxford) 2005).

Multiple fibrotic pathways are activated in scleroderma/SSc for reasons that are not completely understood. The pathogenesis of fibrosis in scleroderma/SSc involves a complex set of interactions involving immune activation, microvascular damage and the activation of fibroblasts. Scleroderma/SSc is characterized by excessive deposition of collagen in the skin and other involved organs and abnormalities of blood vessels (Jimenez, S. A., et al. Rheum. Dis. Clin. North Am. 1996 November; 22(4):647-74; Sakkas, L. I. Autoimmunity 2005 March; 38(2):113-6). TGFβ1, a multifunctional cytokine, is an indirect mitogen for human fibroblasts, which through upregulating PDGF, is capable of inducing normal fibroblasts into a pathogenic myofibroblast phenotype that mediates ECM (collagen) accumulation (Mauch, C., et al. J. Invest. Dermatol. 1993 January; 100(1):925-965; Hummers, L. K., et al. J. Rheumatol. 2009 March; 36(3):576-82). The ubiquitous growth factors TGFβ and PDGF are the most potent proteins involved in fibroblast proliferation, collagen gene expression and connective tissue (collagen) accumulation (Antoniades, H. N. Baillieres Clin. Endocrinol. Metab. 1991 December; 5(4):595-613). Numerous other cytokines including VEGF, as well as cell-matrix interactions, also modify collagen expression and can influence the effects of TGFβ1 and PDGF (Trojanowska, M. Rheumatology (Oxford) 2008 October; 47 Suppl 5:v2-4). Persistent overproduction of collagen and other connective tissue results in excessive accumulation of ECM components leading to the formation of scar tissue (fibrosis) in the skin and other organs and is responsible for the progressive nature of scleroderma/SSc (Mauch, C. Rheum. Dis. Clin. North Am. 1990 February; 16(1):93-107). This leads to thickness and firmness of involved areas. Overall, the pathogenic cascade at different stages of scleroderma/SSc may have autoimmune, inflammatory, fibrotic and vascular components with systemic fibrosis and vasculopathy. Studies indicate that severe fibrosis and abnormal vascular remodeling were detected and the systemic vasculopathy is a hallmark in the pathogenesis of scleroderma/SSc (Yamamoto, T. Autoimmune mechanisms of scleroderma and a role of oxidative stress. 2011 January; 2(1):4-10).

Other findings suggest that the pathology of scleroderma/SSc is driven by PDGF, and elevated expression of PDGF and its receptors have been found in scleroderma skin and lung tissues (Mauch 1993). Studies indicate that abnormal vascular remodeling with significant elevations of VEGF and PDGF in SSc patients and systemic vasculopathy is the most striking feature of SSc (Ou, X. M., et al. Int. Immunopharmacol. 2009 January; 9(1):70-9; Pytel, D., et al. Anticancer Agents Med. Chem. 2009 January; 9(1):66-76). PDGF and VEGF, together with their cognate receptors, have been shown to be upregulated in the skin of SSc patients.

The clinical management of patients with scleroderma/SSc remains a challenge and involves several therapeutic approaches. Methotrexate, cyclophosphamide, calcium channel blockers, ACE inhibitors, prostacyclin analogues and D-penicillamine are the most widely studied treatments for SSc. IV gamma globulins, mycophenolate mophetil, rituximab, fluoxetine, pirfenidone, relaxin, halofuginone, and anti-TGF-beta antibodies await more solid data, and side effects are common (Sapadin, A. N., et al. Arch. Dermatol. 2002 January; 138(1):99-105; Stummvoll G. H. Acta Med. Austriaca 2002; 29(1):14-9; Zandman-Goddard, G., et al. Clin. Dev. Immunol. 2005; 12(3):165-73; Grassegger, A., et al. Clin. Exp. Dermatol. 2004 November; 29(6):584-8; Nash, R. A., et al. Blood 2007; 110(4):1388-96; Gavino, E. S. and Furst D. E. BioDrugs 2001; 15(9):609-14; Au, K., et al. Curr. Rhemuatol. Rep. 2009 April; 11(2):111). A combination of immunosuppressive agents and imatinib was tested in SSc patients for treating SSc-related lung disease (Kay, J. Arthritis Rheum. 2008 August; 58(8):2543-8; Sabnani, I. Rheumatology (Oxford) 2009 January; 58(1):49-52). Overall, the result of current research is mixed, with limited positive reports.

### Gastrointestinal Diseases, Disorders, and Conditions

In some embodiments, provided methods are useful for treating gastrointestinal diseases, disorders, and conditions. In some embodiments, provided methods are useful for treating gastrointestinal fibrosis (e.g., fibrosis of esophagus, stomach, intestines, and/or colon). In some embodiments, provided methods are useful for treating gastrointestinal fibrosis secondary to, or otherwise associated with, an underlying indication. In some embodiments, provided methods are useful for treating inflammatory bowel disease (e.g., ulcerative colitis or Crohn's disease), e.g., treating gastrointestinal fibrosis associated with inflammatory bowel disease.

In some embodiments, a disease to be treated by methods of the present disclosure is inflammatory bowel disease (IBD). IBD is an inflammatory condition that comprises both ulcerative colitis (UC) and Crohn's disease (CD). While UC affects the entire colon, CD typically affects the ileum but can occur to any part of GI tract. IBD can manifest as acute or chronic colitis, characterized by recurrent intestinal inflammation accompanied by diarrhea and abdominal pain (Arivarasu, N., et al. Tissue Barriers 2018; 6(2):e1463897; Ponder, A. and Long, M. D. Clin. Epidemiol. 2013; 5:237-47). Recurring bouts of inflammation can lead to tissue remodeling and is a serious presentation in IBD and a major cause of morbidity, often requiring hospitalization and surgical intervention (Wendelsdorf, K., et al. J. Theor. Biol. 2010 Jun. 21; 264(4):1225-39; Fornaro, R., et al. J. Dig. Dis. 2015 October; 16(10):558-67).

Incidence of IBD is increasing worldwide and is an expanding global health problem (Amosy, E., et al. Clin. Med. Insights Gastroenterol. 2013; 6:33-47). An estimated 2.5-3 million people in Europe are affected by IBD (Burisch, J., et al. J. Crohns Colitis 2013 May; 7(4):322-37). According to the Centers for Disease Control and Prevention (CDC), 3.1 million adults in this country were diagnosed with IBD in 2015, a substantial increase from the ˜1.4 million adults diagnosed per 2008 reports (www.cdc.gov/IBD; www.cdc.gov/ibd/pdf/inflammatory-bowel-disease-an-expensive-disease.pdf). IBD accounts for ˜1,300,000 physician visits and ˜92,000 hospitalizations each year in the United States. Of these, 75% patients diagnosed with CD and 25% patients diagnosed with UC and require surgery. Risk factors associated with IBD include environmental, genetic and immunologic factors (Abegunde, A. T., et al. World J. Gastroenterol. 2016 Jul. 21; 22(27):6296-6317; Frolkis, A., et al. Can. J. Gastroenterol. 2013 March; 27(3):e28-24).

IBD is a major cause of morbidity in patients and is a major consumer of the health care budget. A European study estimated that direct healthcare costs for IBD in Europe are ˜5 billion Euros/year (Burisch 2013). In 2008, CDC reports indicate that direct treatment costs with IBD were estimated ˜$6.3 billion and indirect costs were estimated to cost an additional $5.5 billion (www.cdc.gov/IBD). A study in 2017 indicated that the annual direct and indirect costs related to ulcerative colitis (UC) are estimated to be as high as €12.5-29.1 billion in Europe and US$8.1-14.9 billion in the USA (Ungaro, R., et al. Lancet 2017 Apr. 29; 389(10080):1756-1770). Thus, IBD is an expensive disease without a cure.

IBD is an autoimmune disease with excessive activation of the adaptive immune response. Various factors including genetic factors alter the intestinal flora and trigger an inflammatory reaction, activate T cells, B cells, mast cells, macrophages and microglia, smooth muscle cells and fibroblasts in the colon, inducing mucosal disruption (Hildner, K., et al. Dig. Dis. 2016; 34Suppl 1:40-7; Curciarello, R., et al. Front Med. (Lausanne) 2017 Aug. 7; 4:126). Epithelial and endothelial damage release chemotactic factors promoting recruitment and activation of inflammatory cells, and release various cytokines including TNFα, and activate fibroblasts via TGFβ1. Activated fibroblasts, i.e. myofibroblasts, secrete growth factors including platelet derived growth factor (PDGF) and vascular endothelial growth factor (VEGF) (Scaldaferri, et al. Gastroenterology 2009 February; 136(2):585-95.e5). Studies indicate that angiogenesis is also an important part of IBD pathogenesis in the colon of IBD patients. In fact, Alkim, et al. demonstrated enhanced microvessel density in the intestinal tissue of both UC and CD patients, which correlated both the level of local VEGF expression and disease activity (Int. J. Inflam. 2015; 2015:970890).

Anti-inflammatory drugs, including 5-aminosalicylic acid (5-ASA)-based preparations, are often the first line of therapy in IBD (Segars, L. W., et al. Clin. Pharm. 1992 June; 11(6):514-28). Anti-TNFα antibodies such as infliximab and adalimumab are also being used. Nevertheless, patients treated with adalimumab are at increased risk for serious infections and lymphoma (Dulai, P. S., et al. Clin. Gastroenterol. Hepatol. 2014 September; 12(9):1443-51). Corticosteroids, other immuno-suppressants, and antibiotics exhibit multiple side effects with relatively poor treatment responses (Kopylov, U., et al. Adv. Gastroenterol. 2016 July: 9(4):513-26; Waljee, A. K., et al. PLoS One 2016 Jun. 23; 11(6):e0158017; Cosnes, J., et al. Gut 2005; 54:237-241).

Studies indicate that PDGF and its receptors are highly expressed in areas of ongoing inflammation and fibrosis in IBD (Zeisberg, M. and Kalluri, R. Am. J. Physiol. Cell Physiol. 2013 Feb. 1; 304(3):C216-C225). PDGF activates fibroblasts and IBD-fibroblasts proliferate more rapidly than normal fibroblasts; collagen secretion from IBD patients' fibroblasts was increased compared to collagen secretion by normal fibroblasts. IBD is also associated with increased circulating PDGF and the level of this growth factor has been reported to correspond with disease severity (Andrae, J., et al. Genes Dev. 2008 May 15; 22(10):1276-1312).

Studies indicate that angiogenesis as a novel component of IBD pathogenesis and angiogenic activity is increased in IBD patients. Serum VEGF levels were significantly higher in IBD patients compared to controls in several studies. Griga et al. demonstrated that sources of increased serum VEGF were from inflamed intestinal tissue of IBD patients (Scand. J. Gastroenterol. 1998 May; 33(5):504-8; Hepatogastroenterology 2002 January-February; 49(43):116-23; Hepatogastroenterology 1999 March-April; 46(26):920-3; Eur. J. Gastroenterol. Hepatol. 1999 February; 11(2):175-9). Furthermore, they found VEGF expression was markedly increased in the inflamed mucosa of both CD and UC patients, when compared with normal mucosa of the same patient. Studies also showed that VEGF expression was increased in colon and was higher across all IBD groups (both CD and UC) when compared with healthy controls. Scaldaferri, et al. (2009) reported that VEGF receptor (VEGFR/KDR) levels were increased in intestinal samples of IBD patients, and in mice with experimental colitis.

Other Indications Associated with Fibrosis

In some embodiments, provided methods are useful for treating other diseases, disorders, and conditions associated with fibrosis. In some embodiments, provided methods are useful for treating cardiac fibrosis and/or fibrosis associated with cardiovascular system. In some embodiments, provided methods are useful for treating cardiac fibrosis secondary to, or otherwise associated with, an underlying indication. In some embodiments, provide methods are useful for treating cardiac and/or cardiovascular fibrosis associated with ischemic heart disease, myocardial ischemia, athereosclerosis, myocardial perfusion (e.g., as a consequence of chronic cardiac ischemia or myocardial infarction), vascular occlusion, or restenosis.

In some embodiments, a disease to be treated by methods of the present disclosure is ischemic heart disease. Ischemic heart disease is a leading cause of morbidity and mortality in the US, afflicting millions of Americans each year at a cost expected to exceed $300 billion/year. Numerous pharmacological and interventional approaches are being developed to improve treatment of ischemic heart disease including reduction of modifiable risk factors, improved revascularization procedures, and therapies to halt progression and/or induce regression of atherosclerosis. Furthermore, atherosclerosis comprises a fibrotic component.

In some embodiments, provided methods are useful for treating fibrosis associated with central nervous system (CNS) and/or one or more CNS-related diseases, disorders, or conditions. In some embodiments, provided methods are useful for treating CNS-associated fibrosis secondary to, or otherwise associated with, an underlying indication. In some embodiments, provided methods are useful for treating fibrosis associated with cerebral infarction, stroke, or amyotrophic lateral sclerosis.

In some embodiments, provided methods are useful for treating fibrosis associated with musculoskeletal system and/or one or more musculoskeletal diseases, disorders, or conditions. In some embodiments, provided methods are useful for treating musculoskeletal-associated fibrosis secondary to, or otherwise associated with, an underlying indication. In some embodiments, provided methods are useful for treating fibrosis associated with muscular dystrophy.

In some embodiments, provided methods are useful for treating pancreatic fibrosis. In some embodiments, provided methods are useful for treating pancreatic fibrosis secondary to, or otherwise associated with, an underlying indication. In some embodiments, provided methods are useful for treating fibrosis associated with pancreatitis.

### EXAMPLES

The following Examples are not intended to limit the scope of any claim. The following non-limiting Examples are provided to further illustrate the present teachings. Those of skill in the art, in light of the present application, will appreciate that many changes can be made in the specific embodiments that are provided herein and still obtain a like or similar result without departing from the spirit and scope of the present teachings.

### Materials & Methods

### High Performance Liquid Chromatography (HPLC)

High performance liquid chromatography (HPLC) was performed according to the conditions described in Table 1:

TABLE 1

| System | Agilent1100 series liquid chromatograph or equivalent | | |
|---|---|---|---|
| Column | X-Bridge C18; 50 × 4.6 mm; 5 μm particle size | | |
| Mobile Phase A | 0.1% Trifluoroacetic acid in water | | |
| Mobile Phase B | 0.1% Trifluoroacetic acid in acetonitrile | | |
| Flow Rate | 1.0 mL · min⁻¹ | | |
| Injection Volume | 5 μL | | |
| Detection | UV detection (254 nm) | | |
| Column | 40° C. | | |
| Temperature | | | |
| | Time (min) | % A | % B |
| Gradient | 0 | 50 | 50 |
| | 10 | 10 | 70 |
| | 11 | 0 | 100 |
| | 12 | 0 | 100 |
| | 14 | 50 | 50 |
| | 15 | 50 | 50 |

| | |
|---|---|
| Sample | 0.5 mg · mL⁻¹ in acetonitrile/methanol (1:1) |
| Preparation | |

### Solution State Proton NMR

¹H NMR spectra were collected using a JEOL ECX 400 MHz spectrometer equipped with an auto-sampler. The samples were dissolved in a suitable deuterated solvent for analysis. The data were acquired using Delta NMR Processing and Control Software version 4.3.

### X-Ray Powder Diffraction (XRPD)

X-Ray Powder Diffraction patterns were collected on a PANalytical diffractometer using Cu Kα radiation (45 kV, 40 mA), θ-θgoniometer, focusing mirror, divergence slit (½″), soller slits at both incident and divergent beam (4 mm) and a PIXcel detector. The software used for data collection was X'Pert Data Collector, version 2.2f and the data were presented using X'Pert Data Viewer, version 1.2d.

XRPD patterns were acquired under ambient conditions via a transmission foil sample stage (polyimide—Kapton, 12.7 μm thickness film) under ambient conditions using a PANalytical X'Pert PRO. The data collection range was 2.994-35° 20 with a continuous scan speed of 0.202004° s⁻¹.

### Differential Scanning Calorimetry (DSC)

DSC data were collected on a PerkinElmer Pyris 6000 DSC equipped with a 45 position sample holder. The instrument was verified for energy and temperature calibration using certified indium. A predefined amount of the sample, e.g., 0.5-3.0 mg, was placed in a pin holed aluminum pan and heated at 20° C.·min⁻¹ from 30° C. to 350° C., or varied as experimentation dictated. A purge of dry nitrogen at 20 mL·min⁻¹ was maintained over the sample. The instrument control, data acquisition and analysis were performed with Pyris Software v11.1.1 Revision H.

### Thermogravimetric Analysis (TGA)

TGA data were collected on a PerkinElmer Pyris 1 TGA equipped with a 20 position auto-sampler. The instrument was calibrated using a certified weight and certified Alumel and Perkalloy for temperature. A predefined amount of the sample, e.g., 1-5 mg, was loaded onto a pre-tared aluminum crucible and was heated at 20° C.·min⁻¹ from ambient temperature to 400° C. A nitrogen purge at 20 mL·min⁻¹ was maintained over the sample. The instrument control, data acquisition and analysis were performed with Pyris Software v11.1.1 Revision H.

### Dynamic Vapor Sorption (DVS)

Sorption isotherms were obtained using a Hiden Isochema moisture sorption analyzer (model IGAsorp), controlled by IGAsorp Systems Software V6.50.48. The sample was maintained at a constant temperature (25° C.) by the instrument controls. The humidity was controlled by mixing streams of dry and wet nitrogen, with a total flow of 250 mL·min⁻¹. The instrument was verified for relative humidity content by measuring three calibrated Rotronic salt solutions (10%, 50%, 88%). The weight change of the sample was monitored as a function of humidity by a microbalance (accuracy+/−0.005 mg). A defined amount of sample was placed in a tared mesh stainless steel basket under ambient conditions. A full experimental cycle typically consisted of three scans (sorption, desorption and sorption) at a constant temperature (25° C.) and 10% RH intervals over a 0-90% range (60 minutes for each humidity level).

### Karl Fischer Titration

Water content in a sample was determined using a Mettler Toledo Volumetric Karl Fischer Titrator. The titrant was HYDRANAL composite 5, and the solvent was HYDRANAL Methanol dry. A sample mass of 0.2 g was charged and mixed for 600 seconds.

### Example 1: Synthesis of Compound 1

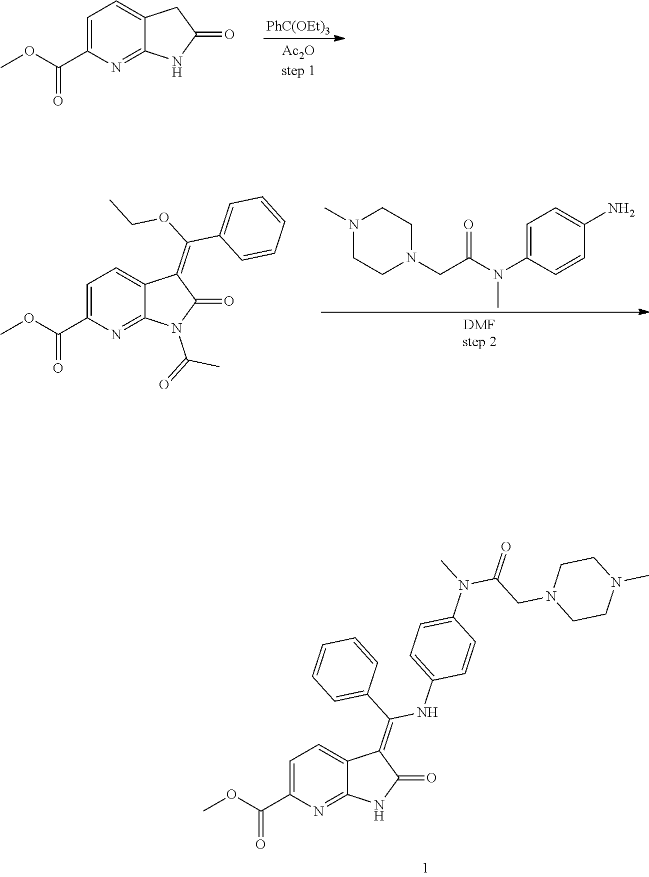

Step 1. To a solution of methyl 2-oxo-2,3-dihydro-1H-pyrrolo[2,3-b]pyridine-6-carboxylate (1 g, 5.20 mmol) in Ac₂O (10 mL) was added triethyl orthobenzoate (3.40 g, 15.59 mmol) at RT and the mixture was heated to reflux for 3 h. The reaction mixture was evaporated and the resultant residue was purified by silica gel column chromatography using 5% CH₃OH in dichloromethane as eluent to afford (E)-methyl 1-acetyl-3-(ethoxy(phenyl)methylene)-2-oxo-2,3-dihydro-1H-pyrrolo[2,3-b]pyridine-6-carboxylate as an orange solid. ¹H NMR (CDCl₃, 500 MHz): δ 8.25 (d, J=12.1 Hz, 1H), 8.04 (d, J=12.1 Hz, 1H), 7.53-7.60 (m, 3H), 7.38-7.45 (m, 2H), 4.40 (q, J=7.1 Hz, 2H), 3.99 (s, 3H), 2.63 (s, 3H), 1.42 (t, J=7.1 Hz, 3H).

Step 2. To a solution of (E)-methyl 1-acetyl-3-(ethoxy(phenyl)methylene)-2-oxo-2,3-dihydro-1H-pyrrolo[2,3-b]pyridine-6-carboxylate (2.6 g, 7.10 mmol) in DMF (5 mL) was added N-(4-aminophenyl)-N-methyl-2-(4-methylpiperazin-1-yl)acetamide (1.94 g, 7.43 mmol) at RT and the reaction mixture was heated to 110° C. and stirred for 1 h. The reaction mixture was allowed to cool to RT, treated with piperidine (3 mL) and stirred for 30 min. The reaction mixture was evaporated and the resultant residue was purified by silica gel column chromatography using 5% CH₃OH in dichloromethane as eluent to afford (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate as yellow solid. MS (ES+): m/z 541.1 (MH⁺).

### Example 2: Preparation & Characterization of Solid Forms of Compound 1 Hydrochloride

### Example 2.1: Compound 1 Hydrochloride Form A

Lots comprising Compound 1 Hydrochloride Form A were generally considered to be one of two types. Type 1 Lots comprised Compound 1 Hydrochloride Form A and excess hydrochloric acid. Type 2 Lots comprised Compound 1 Hydrochloride Form A substantially free of excess hydrochloric acid. Although Type 1 Lots contained excess hydrochloric acid, it will be appreciated that the solid form of Compound 1 Hydrochloride Form A was substantially unchanged between the two types, as indicated by results of various characterization techniques described herein.

### Example 2.1.1: Compound 1 Hydrochloride Form A (Type 1 Lot)

A representative procedure for obtaining Type 1 Lots of Compound 1 Hydrochloride Form A is as follows. The quantities of materials used are approximate and may be increased or decreased in unison to obtain a larger or smaller lot size. Conditions such as time or temperature are approximate and may be used as targets.

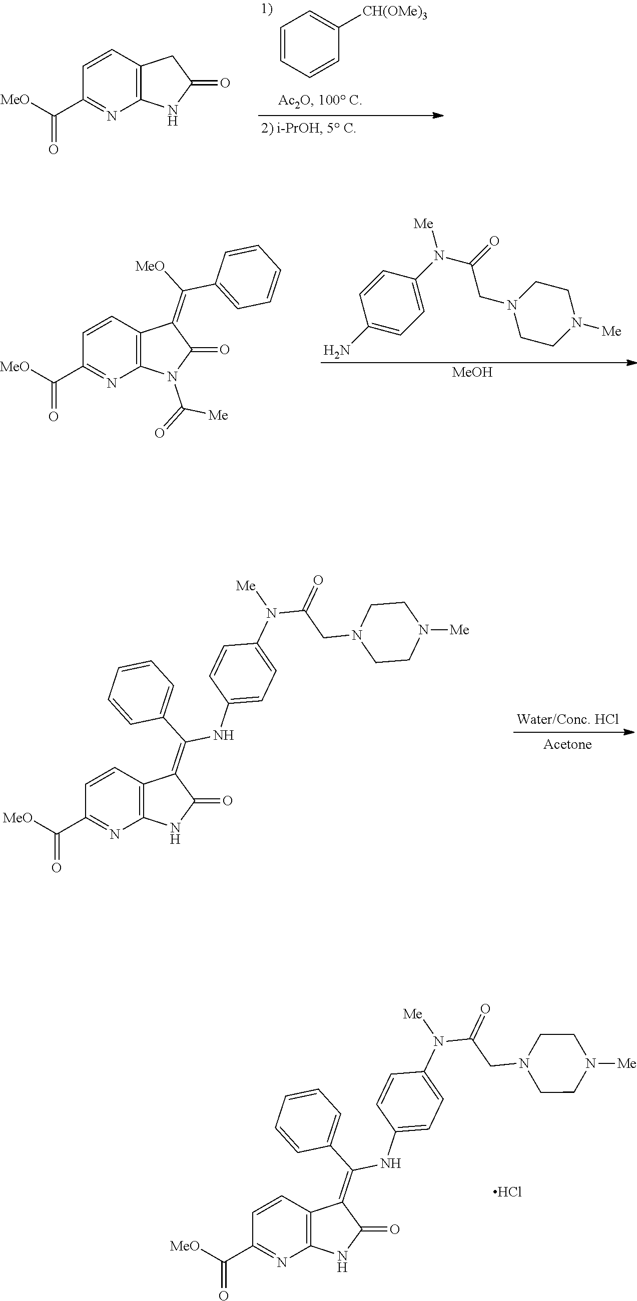

Step 1: Acetic anhydride (5.40 kg) and methyl 2-oxo-2,3-dihydro-1H-pyrrolo[2, 3-b]pyridine-6-carboxylate (1.0 kg) were added to a reactor at room temperature and stirred to combine. Trimethylorthobenzoate (1.90 kg) was added to the reaction mixture. The mixture was then heated to 105° C. and stirred for 1 hr. The reaction was cooled to 40° C. and isopropyl alcohol (3.14 kg) was added. The reaction was cooled further to 5° C. and stirred for 4 hr. The mixture was then filtered, and the product washed with isopropyl alcohol twice. The product was spin dried for 1 hr and for 4 hr at 50° C. under vacuum to give methyl (E)-1-acetyl-3-(methoxy(phenyl)methylene)-2-oxo-2,3-dihydro-1H-pyrrolo[2,3-b]pyridine-6-carboxylate.

Step 2: Methanol (7.12 kg), methyl (E)-1-acetyl-3-(methoxy(phenyl)methylene)-2-oxo-2,3-dihydro-1H-pyrrolo[2,3-b]pyridine-6-carboxylate (1.0 kg), and N-(4-aminophenyl)-N-methyl-2-(4-methylpiperazin-1-yl) acetamide (0.78 kg) in methanol (0.79 kg) were added to a reactor at room temperature and stirred to combine. The reaction mixture was then heated to 63° C. and stirred for 4 hrs. The reaction mixture was cooled to 5° C. and stirred for 4 hrs. The reaction mixture was then filtered, and the product washed with methanol (1.98 kg). The product was spin dried for 1 hr and for 4 hr at 50° C. under vacuum to give methyl (Z)-3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate.

Step 3: Purified water (2.50 kg) and methyl (Z)-3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate (1.0 kg) were added to a reactor at room temperature and stirred to combine. Acetone (1.975 kg) was then added, followed by iron-free HCl (1.20 mol equiv.). The mixture was stirred for 1 hr at 30° C., and then filtered through a micron filter and washed with purified water (0.2 kg). The washings were transferred into a reactor and acetone was added (21.72 kg). The mixture was stirred for 1 h at 30° C., then cooled to 5° C., and stirred for 5 h. The product was isolated in centrifuge and washed with acetone (1.58 kg). The product was spin dried for 1 hr and for 8 hrs at 50° C. under vacuum to give Compound 1 as a hydrochloride salt.

The solid was isolated and characterized by XRPD, DSC, TGA, DVS, Karl Fischer titration, and elemental analysis.

The XRPD pattern of Compound 1 Hydrochloride Form A from a Type 1 Lot is shown in FIG. 2, and the corresponding data are summarized below:

| Pos. [°2Th.] | d-spacing [Å] | Rel. Int. [%] |
|---|---|---|
| 5.30 | 16.66 | 96.92 |
| 5.69 | 15.54 | 20.16 |
| 9.77 | 9.06 | 13.24 |
| 10.66 | 8.30 | 52.51 |
| 11.55 | 7.66 | 30.17 |
| 12.74 | 6.95 | 26.26 |
| 13.13 | 6.74 | 19.55 |
| 13.98 | 6.33 | 13.73 |
| 15.34 | 5.77 | 13.72 |
| 15.68 | 5.65 | 30.65 |
| 16.04 | 5.53 | 35.82 |
| 16.54 | 5.36 | 23.13 |
| 16.99 | 5.22 | 9.38 |
| 17.74 | 5.00 | 20.24 |
| 18.89 | 4.70 | 20.54 |
| 20.14 | 4.41 | 16.27 |
| 20.97 | 4.24 | 100.00 |
| 21.11 | 4.21 | 91.21 |
| 21.68 | 4.10 | 15.37 |
| 22.09 | 4.02 | 16.81 |
| 22.89 | 3.89 | 32.37 |
| 24.06 | 3.70 | 13.82 |
| 24.97 | 3.57 | 8.13 |
| 25.73 | 3.46 | 20.51 |
| 27.08 | 3.29 | 6.49 |
| 27.84 | 3.21 | 22.33 |
| 28.31 | 3.15 | 12.25 |
| 29.92 | 2.99 | 8.36 |
| 30.96 | 2.89 | 15.49 |
| 31.81 | 2.81 | 8.70 |

As shown by the DSC curve in FIG. 3, the sample displayed an onset of 109.8° C. with peaks at 111.4° C. and 121.1° C., followed by a second multi-modal endotherm with an onset temperature of 272.8° C. and a main peak temperature of 278.6° C. FIG. 3 also shows the TGA curve, which shows a weight loss of 8.5% up to 120° C., considered to relate to the loss of water from the solid, followed by a further weight reduction of 2.1% above 200° C., considered to relate to the loss of more tightly bound water. Thus, a total loss of water of 10.6% was observed. A Karl Fischer measurement indicated the solid contained 9.1% water, which correlated well with the results from TGA. Based on these results, Form A was considered to be a trihydrate under these conditions.

DVS analysis is shown in FIG. 4. Over the humidity range, the solid absorbed 13.4 wt % water, which indicated that the salt was hydrating as much as four molar equivalents of water and displaying sponge-like behavior even at low RH. Up to 30% RH, rapid water uptake to 8.5 wt % was observed, which continued steadily up to 90% relative humidity. The water uptake was reversible with evidence of minor hysteresis during desorption. XRPD patterns across the humidity range were substantially identical to the input material over the course of DVS analysis, confirming that Form A remained despite high levels of water uptake over the humidity range. Based on DVS analysis, Form A was considered to be a channel-type hydrate.

Elemental analysis indicated that Type 1 Lots of Compound 1 Hydrochloride Form A contained approximately 15% excess HCl, assuming 10.6% water content (determined from TGA described above). Results are summarized in Table 2.

TABLE 2

| | C | H | N | Cl |
|---|---|---|---|---|
| % Found | 55.21 | 5.91 | 12.68 | 6.31 |
| % Theory (1:1 HCl) | 57.04 | 5.70 | 13.31 | 5.61 |
| % Theory (1.15:1 HCl) | 55.27 | 6.32 | 12.89 | 6.25 |

### Example 2.1.2: Type 2 Lots of Compound 1 Hydrochloride Form A

A representative procedure for obtaining Type 2 Lots of Compound 1 Hydrochloride Form A is as follows. Compound 1 Free Base (1.5 g) was charged into a flask. Deionized water (22.5 mL, 15 volumes) was added and the resulting yellow suspension heated to 50° C. Concentrated hydrochloric acid (231 μL, 1 equiv) was added which clarified the suspension giving a dark yellow solution. The mixture was cooled to room temperature and IPA (20 mL) was added. The mixture was concentrated in vacuo until <5 mL of water remained. IPA (20 mL) was added, and the thin yellow suspension was seeded with Compound 1 Hydrochloride Form A (obtained as described in Example 2.1.1 or elsewhere herein) and matured at room temperature for 18 hours. The solid was isolated by filtration and dried in vacuo at 45° C. for 18 hours to give Compound 1 Hydrochloride Form A (Type 2 Lot) as a yellow solid (1.22 g, 76% yield).

The XRPD pattern of Compound 1 Hydrochloride Form A (Type 2 Lot) is shown in FIG. 5, and the corresponding data are summarized below:

| Pos. [°2Th.] | d-spacing [Å] | Rel. Int. [%] |
|---|---|---|
| 5.28 | 16.74 | 53.16 |
| 5.76 | 15.34 | 11.29 |
| 10.63 | 8.33 | 39.94 |
| 11.54 | 7.67 | 24.64 |
| 12.73 | 6.95 | 26.80 |
| 13.13 | 6.74 | 15.41 |
| 14.08 | 6.29 | 13.06 |
| 15.34 | 5.78 | 10.48 |
| 15.64 | 5.67 | 26.62 |
| 16.00 | 5.54 | 27.14 |
| 16.55 | 5.36 | 17.61 |
| 17.05 | 5.20 | 14.64 |
| 17.78 | 4.99 | 17.27 |
| 18.86 | 4.71 | 19.51 |
| 19.08 | 4.65 | 13.22 |
| 20.16 | 4.41 | 17.67 |
| 20.68 | 4.30 | 14.69 |
| 20.98 | 4.23 | 100.00 |
| 21.62 | 4.11 | 17.55 |
| 22.05 | 4.03 | 21.34 |
| 22.82 | 3.90 | 36.58 |
| 23.97 | 3.71 | 16.38 |
| 24.94 | 3.57 | 7.90 |
| 25.23 | 3.53 | 7.88 |
| 25.61 | 3.48 | 13.63 |
| 26.58 | 3.35 | 4.27 |
| 27.01 | 3.30 | 5.44 |
| 27.78 | 3.21 | 24.33 |
| 29.89 | 2.99 | 7.02 |
| 30.51 | 2.93 | 4.79 |
| 30.91 | 2.89 | 12.58 |
| 31.72 | 2.82 | 10.55 |
| 33.39 | 2.68 | 2.07 |

As shown by the DSC curve in FIG. 6, the sample displayed an onset of 144.0° C. with a peak at 158.6° C., followed by a second endotherm with an onset of 268.4° C. and a peak at 274.4° C. FIG. 6 also shows the TGA curve, which shows a weight loss of 3.76% up to 100° C.

### Example 2.2: Compound 1 Hydrochloride Form B

Compound 1 Hydrochloride Form B was obtained according to the following procedure. Compound 1 Hydrochloride Form A (Type 1 Lot) (200 mg) was charged into a reaction tube. Methanol (5 mL, 25 volumes) was added. The mixture was equilibrated at 50° C. for 6 hours and then cooled to room temperature and seeded with ˜0.1 wt % of Compound 1 Hydrochloride Form B obtained from the Solvent Slurry Screen using Compound 1 Hydrochloride Form A (Type 1 Lot) described in Example 5 below and equilibrated for 48 hours. Solids were isolated by filtration and dried in vacuo at 45° C. for 18 hours to give Compound 1 Hydrochloride Form B (131 mg, 66% recovery).

The XRPD pattern of Compound 1 Hydrochloride Form B is shown in FIG. 7, and the corresponding data are summarized below:

| Pos. [°2Th.] | d-spacing [Å] | Rel. Int. [%] |
|---|---|---|
| 6.02 | 14.68 | 37.80 |
| 10.07 | 8.78 | 64.08 |
| 10.46 | 8.46 | 100.00 |
| 13.02 | 6.80 | 30.84 |
| 14.13 | 6.27 | 10.08 |
| 14.99 | 5.91 | 30.56 |
| 15.35 | 5.77 | 8.60 |
| 16.27 | 5.45 | 9.18 |
| 17.65 | 5.02 | 55.88 |
| 18.17 | 4.88 | 40.50 |
| 18.88 | 4.70 | 18.35 |
| 19.63 | 4.52 | 66.35 |
| 20.27 | 4.38 | 53.88 |
| 20.90 | 4.25 | 81.96 |
| 22.41 | 3.97 | 62.84 |
| 23.96 | 3.71 | 49.12 |
| 24.37 | 3.65 | 24.42 |
| 25.45 | 3.50 | 29.66 |
| 26.22 | 3.40 | 12.79 |
| 26.82 | 3.32 | 19.42 |
| 27.90 | 3.20 | 7.29 |
| 28.61 | 3.12 | 4.98 |
| 29.66 | 3.01 | 9.42 |
| 30.30 | 2.95 | 14.91 |
| 31.04 | 2.88 | 27.96 |
| 31.72 | 2.82 | 5.40 |
| 32.91 | 2.72 | 3.77 |
| 34.13 | 2.63 | 3.46 |

As shown by the DSC curve in FIG. 8, the sample displayed a broad endotherm with an onset of 269.5° C. and a peak at 277.8° C., preceded by a minor endotherm with an onset of 246.6° C. and a peak at 252.9° C. FIG. 8 also shows the TGA curve, which shows a weight loss of <1% up to 100° C. and weight loss of 5.2% from 100° C. to 225° C.

### Example 2.3: Compound 1 Hydrochloride Form C

Representative procedures for obtaining Compound 1 Hydrochloride Form C are as follows.

Procedure 1: Compound 1 Hydrochloride Form A (Type 1 Lot) (200 mg) was charged into a reaction tube. TBME (5 mL, 25 volumes) was added. The mixture was equilibrated at 50° C. for 6 hours and then cooled to room temperature and seeded with ˜0.1 wt % of Compound 1 Hydrochloride Form C obtained from the Solvent Slurry Screen using Compound 1 Hydrochloride Form A (Type 1 Lot) described in Example 5 below and equilibrated for 48 hours. Solids were isolated by filtration and dried in vacuo at 45° C. for 18 hours to give Compound 1 Hydrochloride Form C (191 mg, 96% recovery).

Procedure 2: Compound 1 Hydrochloride Form A (Type 1 Lot) (200 mg) was charged into a reaction tube. Ethyl acetate (5 mL, 25 volumes) was added. The mixture was equilibrated at 50° C. for 6 hours and then cooled to room temperature and seeded with ˜0.1 wt % of Compound 1 Hydrochloride Form C obtained from the Solvent Slurry Screen using Compound 1 Hydrochloride Form A (Type 1 Lot) described in Example 5 below and equilibrated for 48 hours. Solids were isolated by filtration and dried in vacuo at 45° C. for 18 hours to give Compound 1 Hydrochloride Form C (170 mg, 85% recovery).

Procedure 3: Compound 1 Hydrochloride Form A (Type 2 Lot) (200 mg) was slurried in TBME (5 mL), heated to 50° C. and matured for 5 hours. The suspension was cooled over approximately 2 hours, filtered, and then dried in vacuo at 45° C. for 18 hours to give Compound 1 Hydrochloride Form C (99% recovery).

Procedure 4: Compound 1 Hydrochloride Form A (Type 1 Lot) (300 mg) was charged to a crystallization tube with acetone (3 mL, 10 volumes). The suspension was equilibrated at 50° C. DI water (800 μL) was added in 100 μL aliquots to achieve dissolution giving a dark yellow solution. The solution was cooled to room temperature and equilibrated for 18 hours. A yellow suspension resulted which was filtered and the solid air dried for 30 minutes. A recovery of 139 mg was recorded. The XRPD pattern of the damp solid indicated Form A. The DSC and TGA showed loss of 11.4% (water content). IC confirmed a 1:1 salt had been isolated, pH 5.1. The solid was subjected to prolonged drying (5 days) at 45° C. and the solids reassessed. XRPD, DSC, and TGA indicated that the solids had fully converted to Form C.

The XRPD pattern of Compound 1 Hydrochloride Form C is shown in FIG. 9, and the corresponding data are summarized below:

| Pos. [°2Th.] | d-spacing [Å] | Rel. Int. [%] |
|---|---|---|
| 5.29 | 16.70 | 19.53 |
| 7.44 | 11.88 | 22.94 |
| 9.38 | 9.43 | 96.08 |
| 9.75 | 9.07 | 40.12 |
| 10.51 | 8.42 | 3.49 |
| 12.97 | 6.83 | 3.61 |
| 14.29 | 6.20 | 33.17 |
| 14.76 | 6.00 | 33.88 |
| 15.66 | 5.66 | 20.53 |
| 16.61 | 5.34 | 46.38 |
| 16.84 | 5.26 | 31.47 |
| 17.26 | 5.14 | 13.35 |
| 17.81 | 4.98 | 10.08 |
| 18.48 | 4.80 | 28.57 |
| 18.87 | 4.70 | 33.60 |
| 19.05 | 4.66 | 34.29 |
| 19.41 | 4.57 | 34.35 |
| 19.90 | 4.46 | 22.39 |
| 20.72 | 4.29 | 25.54 |
| 21.50 | 4.13 | 30.02 |
| 22.38 | 3.97 | 100.00 |
| 23.44 | 3.80 | 26.53 |
| 24.01 | 3.71 | 16.42 |
| 24.73 | 3.60 | 18.44 |
| 25.85 | 3.45 | 8.31 |
| 26.50 | 3.36 | 6.22 |
| 27.44 | 3.25 | 12.35 |
| 27.93 | 3.19 | 31.04 |
| 28.74 | 3.11 | 10.33 |
| 30.04 | 2.97 | 19.07 |
| 30.99 | 2.89 | 5.70 |
| 32.41 | 2.76 | 4.71 |
| 33.89 | 2.65 | 3.67 |

As shown by the DSC curve in FIG. 10, the sample displayed a broad endotherm with an onset of 271.5° C. and a peak at 277.5° C. FIG. 10 also shows the TGA curve, which shows a weight loss of <1% up to 100° C. and weight loss of 1.7% from 100° C. to 225° C.

### Example 2.4: Compound 1 Hydrochloride Form D

A representative procedure for obtaining Compound 1 Hydrochloride Form D is as follows. Compound 1 Hydrochloride Form A (Type 1 Lot) (200 mg) was charged into a reaction tube. Chloroform (5 mL, 25 volumes) was added. The mixture was equilibrated at 50° C. for 6 hours and then cooled to room temperature and seeded with ˜0.1 wt % of Compound 1 Hydrochloride Form D obtained from the Solvent Slurry Screen using Compound 1 Hydrochloride Form A (Type 1 Lot) described in Example 5 below and equilibrated for 48 hours. Solids were isolated by filtration and dried in vacuo at 45° C. for 18 hours to give Compound 1 Hydrochloride Form D (101 mg, 51% recovery).

The XRPD pattern of Compound 1 Hydrochloride Form D is shown in FIG. 11, and the corresponding data are summarized below:

| Pos. [°2Th.] | d-spacing [Å] | Rel. Int. [%] |
|---|---|---|
| 5.29 | 16.72 | 48.83 |
| 9.37 | 9.44 | 90.01 |
| 9.73 | 9.09 | 100.00 |
| 14.16 | 6.25 | 20.26 |
| 15.29 | 5.80 | 14.39 |
| 15.99 | 5.54 | 13.03 |
| 17.35 | 5.11 | 16.47 |
| 18.94 | 4.69 | 35.33 |
| 19.30 | 4.60 | 29.25 |
| 19.59 | 4.53 | 22.59 |
| 19.82 | 4.48 | 19.96 |
| 20.26 | 4.38 | 11.01 |
| 20.89 | 4.25 | 20.23 |
| 21.60 | 4.12 | 25.07 |
| 22.99 | 3.87 | 33.64 |
| 23.39 | 3.80 | 40.65 |
| 23.71 | 3.75 | 41.83 |
| 24.94 | 3.57 | 6.58 |
| 25.70 | 3.47 | 7.93 |
| 26.50 | 3.36 | 7.98 |
| 28.23 | 3.16 | 13.06 |
| 28.96 | 3.08 | 7.56 |
| 30.28 | 2.95 | 16.50 |
| 31.27 | 2.86 | 11.70 |
| 32.37 | 2.77 | 5.16 |

As shown by the DSC curve in FIG. 12, the sample displayed a broad endotherm with an onset of 270.3° C. and a peak at 276.4° C. FIG. 12 also shows the TGA curve, which shows a weight loss of 3.7% up to 100° C. and weight loss of 2.9% from 100° C. to 250° C.

### Example 2.5: Compound 1 Hydrochloride Form E

A representative procedure for obtaining Compound 1 Hydrochloride Form E is as follows. Compound 1 Hydrochloride Form A (Type 1 Lot) (40 mg) was slurried in 4:1 IPA:water (1 mL, 25 volumes). The mixture was thermally cycled at 25° C. and 50° C. for 4 days. Solids were isolated by filtration at 25° C. and dried in vacuo at 45° C. to give Compound 1 Hydrochloride Form E.

The XRPD pattern of Compound 1 Hydrochloride Form E is shown in FIG. 13, and the corresponding data are summarized below:

| Pos. [°2Th.] | d-spacing [Å] | Rel. Int. [%] |
|---|---|---|
| 4.71 | 18.74 | 100.00 |
| 5.61 | 15.75 | 7.37 |
| 7.78 | 11.37 | 54.43 |
| 9.25 | 9.56 | 7.91 |
| 10.30 | 8.59 | 38.04 |
| 13.04 | 6.79 | 46.08 |
| 14.27 | 6.21 | 26.07 |
| 14.88 | 5.95 | 20.60 |
| 15.64 | 5.67 | 72.20 |
| 16.91 | 5.24 | 25.72 |
| 17.67 | 5.02 | 55.47 |
| 18.26 | 4.86 | 27.38 |
| 20.35 | 4.36 | 19.32 |
| 20.69 | 4.29 | 28.71 |
| 21.37 | 4.16 | 13.49 |
| 22.53 | 3.95 | 40.49 |
| 24.20 | 3.68 | 32.98 |
| 25.06 | 3.55 | 65.88 |
| 25.51 | 3.49 | 16.17 |
| 26.31 | 3.39 | 51.46 |
| 29.00 | 3.08 | 20.19 |
| 29.69 | 3.01 | 14.59 |
| 31.28 | 2.86 | 7.04 |
| 32.77 | 2.73 | 6.21 |

As shown by the DSC curve in FIG. 14, the sample displayed one major endotherm with an onset of 275.0° C. and a peak at 281.4° C. FIG. 14 also shows the TGA curve, which shows a weight loss of 5.2% up to 100° C.

### Example 2.6: Compound 1 Hydrochloride Form F

A representative procedure for obtaining Compound 1 Hydrochloride Form F is as follows. Compound 1 Free Base (50 mg) was charged to a crystallization tube and water (1 mL, 20 volumes) was added to give a thick yellow suspension. The solution was heated to 50° C. and concentrated hydrochloric acid (8.47 μL, 1.1 equivalents) was added. The suspension clarified on addition of the acid to give a dark yellow solution. The mixture was cooled to 40° C. and various anti-solvents were added to see if precipitation could be induced on cooling. This was unsuccessful, so the solvent volume was reduced under a gentle stream of N2 to give a thick suspension. IPA (1 mL) was added and the mixture equilibrated for 18 hours. The solids were isolated by filtration and dried in vacuo at 45° C. for 4 hours to give bright yellow solids (42 mg, 79%).

The XRPD pattern of Compound 1 Hydrochloride Form F is shown in FIG. 15, and the corresponding data are summarized below:

| Pos. [°2Th.] | d-spacing [Å] | Rel. Int. [%] |
|---|---|---|
| 5.27 | 16.77 | 10.68 |
| 6.19 | 14.28 | 66.69 |
| 7.30 | 12.12 | 2.61 |
| 9.70 | 9.12 | 81.65 |
| 10.65 | 8.31 | 10.94 |
| 12.09 | 7.32 | 16.64 |
| 12.62 | 7.01 | 14.57 |
| 14.41 | 6.15 | 19.86 |
| 16.00 | 5.54 | 39.05 |
| 16.50 | 5.37 | 10.77 |
| 16.83 | 5.27 | 18.24 |
| 18.73 | 4.74 | 9.14 |
| 19.50 | 4.55 | 43.64 |
| 20.05 | 4.43 | 100.00 |
| 20.71 | 4.29 | 69.71 |
| 21.24 | 4.18 | 54.73 |
| 21.49 | 4.14 | 49.86 |
| 21.95 | 4.05 | 37.94 |
| 22.68 | 3.92 | 74.08 |
| 23.08 | 3.85 | 38.50 |
| 23.71 | 3.75 | 10.42 |
| 24.62 | 3.62 | 15.67 |
| 25.74 | 3.46 | 5.54 |
| 26.89 | 3.32 | 3.95 |
| 27.85 | 3.20 | 9.90 |
| 28.92 | 3.09 | 4.27 |
| 29.88 | 2.99 | 4.71 |
| 30.87 | 2.90 | 7.66 |
| 31.53 | 2.84 | 4.90 |
| 32.49 | 2.76 | 11.45 |
| 33.96 | 2.64 | 4.28 |

As shown by the DSC curve in FIG. 16, the sample displayed an onset of 260.7° C. with a peak at 272.5° C. FIG. 16 also shows the TGA curve, which shows no weight loss.

### Example 2.7: Compound 1 Hydrochloride Form G

A representative procedure for obtaining Compound 1 Hydrochloride Form G is as follows. Compound 1 Hydrochloride Form A (Type 1 Lot) (200 mg) was charged into a reaction tube. 4:1 IPA:water (5 mL, 25 volumes) was added. The mixture was equilibrated at 50° C. for 6 hours and then cooled to room temperature and seeded with ˜0.1 wt % of Compound 1 Hydrochloride Form E obtained from the Solvent Slurry Screen using Compound 1 Hydrochloride Form A (Type 1 Lot) described in Example 5 below and equilibrated for 48 hours. Solids were isolated by filtration and dried in vacuo at 45° C. for 18 hours to give Compound 1 Hydrochloride Form G (91 mg, 46% recovery).

The XRPD pattern of Compound 1 Hydrochloride Form G is shown in FIG. 17, and the corresponding data are summarized below:

| Pos. [°2Th.] | d-spacing [A] | Rel. Int. [%] |
|---|---|---|
| 5.30 | 16.66 | 17.90 |
| 5.75 | 15.37 | 27.52 |
| 9.51 | 9.30 | 1.49 |
| 10.66 | 8.30 | 8.12 |
| 11.56 | 7.65 | 20.33 |
| 11.98 | 7.39 | 5.90 |
| 12.66 | 6.99 | 9.20 |
| 13.18 | 6.72 | 14.26 |
| 13.94 | 6.35 | 7.25 |
| 14.20 | 6.24 | 2.34 |
| 14.62 | 6.06 | 4.81 |
| 15.92 | 5.57 | 13.88 |
| 16.22 | 5.46 | 33.61 |
| 17.16 | 5.17 | 5.50 |
| 17.76 | 5.00 | 6.35 |
| 18.90 | 4.70 | 6.12 |
| 19.40 | 4.57 | 11.08 |
| 20.12 | 4.41 | 17.10 |
| 21.12 | 4.21 | 100.00 |
| 21.67 | 4.10 | 9.10 |
| 22.12 | 4.02 | 8.70 |
| 22.84 | 3.89 | 8.54 |
| 24.09 | 3.69 | 9.60 |
| 25.06 | 3.55 | 7.05 |
| 25.76 | 3.46 | 18.11 |
| 26.25 | 3.39 | 9.32 |
| 27.03 | 3.30 | 3.57 |
| 27.75 | 3.21 | 7.49 |
| 28.31 | 3.15 | 6.69 |
| 29.07 | 3.07 | 5.56 |
| 29.95 | 2.98 | 11.20 |
| 31.03 | 2.88 | 5.38 |
| 31.79 | 2.81 | 3.13 |

As shown by the DSC curve in FIG. 18, the sample displayed a broad endotherm with an onset of 137.6° C. and a peak at 156.9° C., followed by a broad endotherm with an onset of 264.8° C. and a peak at 272.8° C. FIG. 18 also shows the TGA curve, which shows a weight loss of 7.3% up to 100° C.

### Example 2.8: Compound 1 Hydrochloride Form H

A representative procedure for obtaining Compound 1 Hydrochloride Form H is as follows. Compound 1 Free Base (1.0 g) was suspended in IPA/water (19%) (12.33 volumes) and the suspension heated to 50° C. which thinned the suspension. Concentrated HCl (154 μL, 1 eqiuv) was added which clarified the suspension to a deep orange solution. The mixture was equilibrated for 30 minutes before cooling gradually to room temperature. A thin yellow suspension resulted. The solvent volume was reduced by 50% which thickened the suspension. The suspension was matured overnight, following which the solid was filtered to isolate the solids. The solid was air dried for ˜30 minutes giving a yellow solid (damp mass: 1.24 g; pH measured at 5.0).

The XRPD pattern of Compound 1 Hydrochloride Form H is shown in FIG. 19, and the corresponding data are summarized below:

| Pos. [°2Th.] | d-spacing [Å] | Rel. Int. [%] |
|---|---|---|
| 5.52 | 16.00 | 30.88 |
| 9.60 | 9.21 | 38.39 |
| 10.85 | 8.16 | 6.29 |
| 11.46 | 7.72 | 20.35 |
| 12.93 | 6.85 | 4.25 |
| 13.66 | 6.48 | 17.13 |
| 15.60 | 5.68 | 4.31 |
| 16.07 | 5.51 | 4.65 |
| 16.52 | 5.37 | 15.87 |
| 18.39 | 4.82 | 13.18 |
| 19.36 | 4.58 | 25.13 |
| 20.13 | 4.41 | 100.00 |
| 21.60 | 4.11 | 7.86 |
| 22.29 | 3.99 | 9.58 |
| 23.12 | 3.85 | 8.06 |
| 24.16 | 3.68 | 6.92 |
| 25.35 | 3.51 | 5.95 |
| 26.97 | 3.31 | 3.23 |
| 30.03 | 2.98 | 2.77 |
| 31.95 | 2.80 | 2.50 |
| 32.71 | 2.74 | 2.46 |

As shown by the DSC curve in FIG. 20, the sample displayed a broad endotherm with a peak at 115.9° C., followed by another broad endotherm with an onset of 259.9° C. and a peak at 278.5° C. FIG. 20 also shows the TGA curve, which shows a weight loss of 8.2% up to 100° C.

### Example 2.9: Compound 1 Hydrochloride Form I

A representative procedure for obtaining Compound 1 Hydrochloride Form I is as follows. Amorphous Compound 1 (20 mg) was charged into a crystallization tube. MIBK (0.5-1 mL) was added. The mixture was thermally cycled at 25° C. and 50° C. for 4 days. Solids were isolated by filtration at 25° C. All isolated solids were dried in vacuo at 45° C. for 18 hours to give Compound 1 Hydrochloride Form I.

The XRPD pattern of Compound 1 Hydrochloride Form I is shown in FIG. 21, and the corresponding data are summarized below:

| Pos. [°2Th.] | d-spacing [Å] | Rel. Int. [%] |
|---|---|---|
| 5.60 | 15.77 | 15.62 |
| 9.42 | 9.39 | 35.30 |
| 12.24 | 7.23 | 14.45 |
| 13.44 | 6.59 | 6.93 |
| 15.42 | 5.75 | 16.34 |
| 16.06 | 5.52 | 30.47 |
| 16.59 | 5.34 | 34.24 |
| 17.40 | 5.10 | 53.07 |
| 17.60 | 5.04 | 58.96 |
| 17.80 | 4.98 | 48.76 |
| 18.22 | 4.87 | 100.00 |
| 18.95 | 4.68 | 62.23 |
| 19.45 | 4.56 | 32.72 |
| 20.58 | 4.32 | 13.65 |
| 22.85 | 3.89 | 29.76 |
| 23.26 | 3.82 | 34.53 |
| 23.78 | 3.74 | 19.34 |
| 24.24 | 3.67 | 11.29 |
| 24.81 | 3.59 | 12.96 |
| 25.61 | 3.48 | 10.23 |
| 26.48 | 3.37 | 9.13 |
| 28.68 | 3.11 | 8.88 |
| 29.58 | 3.02 | 18.57 |

As shown by the DSC curve in FIG. 22, the sample displayed an onset of 269.5° C. with a peak at 275.7° C. FIG. 22 also shows the TGA curve, which shows a weight loss of 1.9% up to 250° C.

### Example 3: Preparation of Amorphous Compound 1 Hydrochloride

Compound 1 Hydrochloride Form A (Type 2 Lot) (800 mg) was dissolved in 80 mL deionized water and clarified via a 0.45 micon filter frit into a 3 L flask. The yellow solution was snap frozen and dried to a solid under high vacuum to give a yellow solid in quantitative yield (HPLC purity >99%). The solid was characterized by XRPD, DSC, and TGA.

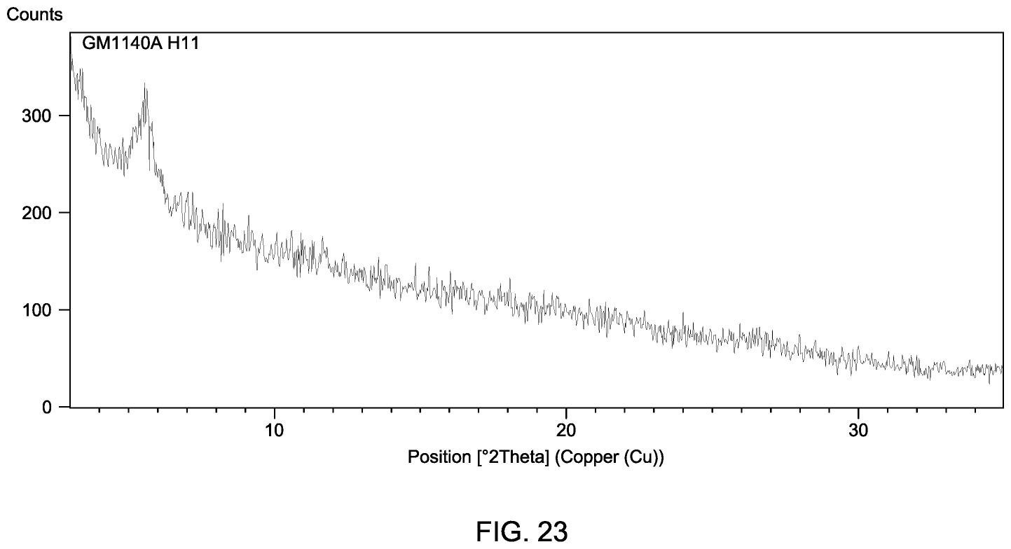

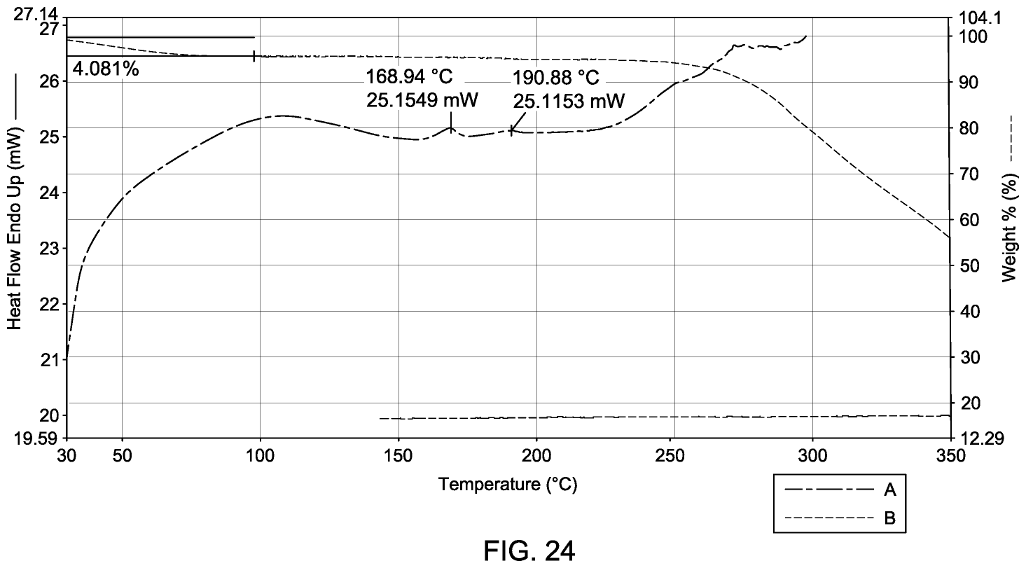

The XRPD pattern of the resulting solid (FIG. 23) confirmed the amorphous nature of the solid. The DSC and TGA thermographs of the solid are shown in FIG. 24. The DSC curve was largely featureless, which is typical of an amorphous solid. The TGA curve showed a weight reduction of 4.1% up to 80° C., related to the loss of loosely bound water.

### Example 4: Preparation & Characterization of Compound 1 Free Base

### Example 4.1: Amorphous Compound 1 Free Base

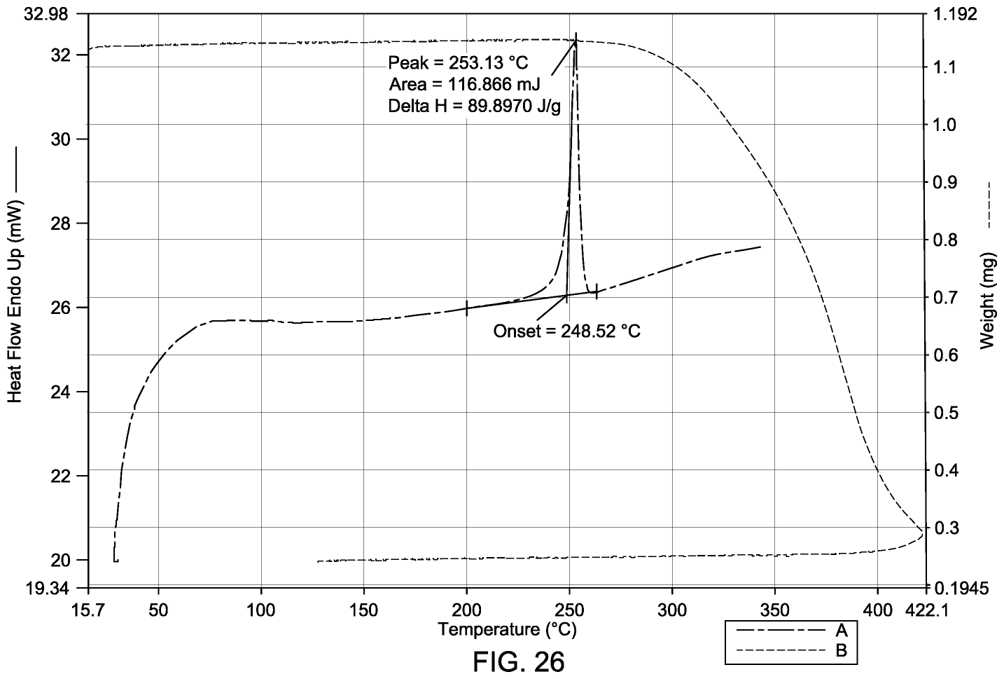

Compound 1 Hydrochloride Form A (Type 1 Lot) (0.5 g) was dissolved in THF/water (4:1) (10 mL, 20 vols) to give a dark yellow solution. Saturated aqueous sodium bicarbonate (5 mL) was added and the mixture stirred for 30 minutes at ambient temperature. Off gassing was noted and formation of a precipitate was observed. The mixture was concentrated in vacuo to remove THF. A thick yellow suspension was obtained. Water (5 mL) was added and the suspension was stirred at ambient temperature for 30 minutes prior to the isolation of the solid by filtration. The solid was dried in vacuo for 18 hours giving a bright yellow solid (415 mg, 89% yield). A successful salt break was indicated by ¹H NMR. The DSC and TGA thermographs of Compound 1 Free Base are shown in FIG. 26. The DSC contained a discrete melt endotherm at 253° C. and the TGA showed no weight reduction prior to decomposition indicating no residual solvent or water was present (FIG. 26).

### Example 4.2: Compound 1 Free Base Form A

Compound 1 Hydrochloride Form A (Type 1 Lot) (5 g) was dissolved in THF/water (4:1) (60 mL, 20 vols) to give a dark yellow solution. Saturated aqueous sodium bicarbonate (30 mL) was added and the mixture stirred for 30 minutes at ambient temperature. Off gassing was observed, and formation of a precipitate was observed. The mixture was concentrated in vacuo to remove the THF. A thick yellow suspension was obtained. Water (15 mL) was added and the suspension was stirred at ambient temperature for 30 minutes prior to the isolation of the solid by filtration. The solid was dried in vacuo for 18 hours giving a bright yellow crystalline solid (4.19 g, 89% yield).

Compound 1 Free Base Form A was also isolated from slurries of Amorphous Compound 1 Free Base in the following solvents: acetonitrile, 1,4-dioxane, ethyl acetate, isopropyl alcohol, methyl ethyl ketone, methyl isobutyl ketone, 2-methyltetrahydrofuran, and tetrahydrofuran.

The XRPD pattern of Compound 1 Free Base Form A is shown in FIG. 25, and the corresponding data are summarized below:

| Pos. [°2Th.] | d-spacing [Å] | Rel. Int. [%] |
|---|---|---|
| 5.55 | 15.92 | 2.89 |
| 6.81 | 12.99 | 9.62 |
| 9.12 | 9.70 | 11.88 |
| 10.56 | 8.38 | 9.73 |
| 12.31 | 7.19 | 13.59 |
| 13.78 | 6.43 | 1.25 |
| 15.35 | 5.77 | 9.78 |
| 16.54 | 5.36 | 6.81 |
| 17.58 | 5.04 | 17.87 |
| 18.12 | 4.90 | 24.62 |
| 19.19 | 4.63 | 100.00 |
| 20.77 | 4.28 | 13.46 |
| 22.78 | 3.90 | 10.57 |
| 24.34 | 3.66 | 10.70 |
| 26.51 | 3.36 | 5.59 |
| 29.45 | 3.03 | 2.43 |
| 30.24 | 2.96 | 2.56 |

### Example 4.3: Compound 1 Free Base Form B

A sample of Amorphous Compound 1 Free Base was slurried in methanol. The XRPD pattern of Compound 1 Free Base Form B is shown in FIG. 27 and the corresponding data are summarized below:

| Pos. [°2Th.] | d-spacing [Å] | Rel. Int. [%] |
|---|---|---|
| 5.60 | 15.79 | 12.07 |
| 7.69 | 11.50 | 29.02 |
| 8.76 | 10.09 | 10.17 |
| 9.47 | 9.34 | 6.56 |
| 10.96 | 8.08 | 32.24 |
| 13.39 | 6.61 | 52.56 |
| 14.64 | 6.05 | 40.63 |
| 16.25 | 5.45 | 26.44 |
| 16.66 | 5.32 | 36.10 |
| 16.88 | 5.25 | 19.09 |
| 17.41 | 5.09 | 20.37 |
| 18.98 | 4.68 | 48.85 |
| 19.98 | 4.44 | 22.03 |
| 20.93 | 4.24 | 100.00 |
| 22.03 | 4.03 | 12.23 |
| 23.20 | 3.83 | 43.00 |
| 23.79 | 3.74 | 27.28 |
| 26.07 | 3.42 | 35.70 |
| 27.25 | 3.27 | 8.10 |
| 27.87 | 3.20 | 12.94 |
| 31.51 | 2.84 | 4.01 |

### Example 5: Polymorph Screening of Compound 1 Hydrochloride

### Example 5.1: Solvent Slurry Screen Using Compound 1 Hydrochloride Form A (Type 1 Lot)

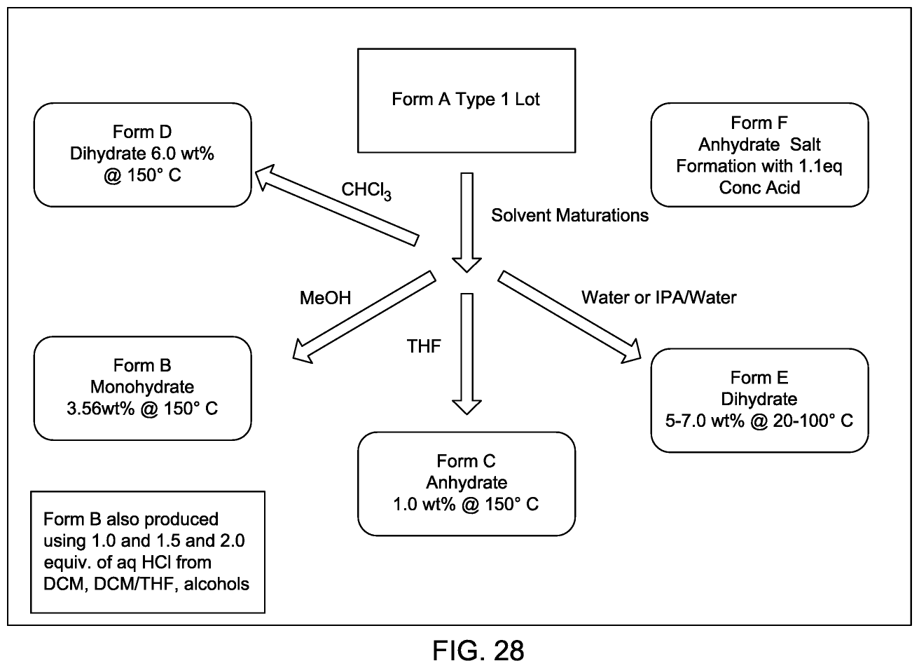

Compound 1 Hydrochloride Form A (Type 1 Lot) (40 mg) was charged into 24 crystallization tubes. The relevant solvent (1 mL, 25 volumes) was added. The mixtures were thermally cycled at 25° C. and 50° C. for 4 days. Solids were isolated by filtration at 25° C. All isolated solids were dried in vacuo at 45° C. Results and observations are summarized in Table 3 and FIG. 28.

TABLE 3

| Solvent | Observations | XRPD |
|---|---|---|
| Cyclohexane | Yellow suspension | Form A |
| CPME | Yellow suspension | Form C |
| Heptane | Yellow suspension | Mixed phase - |
| | | Form A/Form C |
| TBME | Yellow suspension | Form C |
| MIBK | Yellow suspension | Form C |
| MEK | Yellow suspension | Form C |
| Isopropyl acetate | Yellow suspension | Form C |
| Ethyl acetate | Yellow suspension | Form C |
| Toluene | Yellow suspension | Form C |
| THF | Yellow suspension | Form C |
| Methanol | Yellow suspension | Form B |
| Ethanol | Yellow suspension | Form C |
| IPA | Yellow suspension | Form C |
| Acetonitrile | Yellow suspension | Form C |
| Chloroform | Yellow suspension | Form D |
| Acetone | Yellow suspension | Form C |
| DCM | Yellow suspension | Form C |
| Water | Dark yellow solution. | Form E |
| | Additional solid added to give | |
| | suspension. | |
| Ethanol/water | Yellow suspension at 25° C. | Insufficient solid |
| (4:1) | Yellow solution at 50° C. | recovered |
| THF/water (4:1) | Yellow suspension | Form C |
| IPA/water (4:1) | Yellow suspension | Form E |
| Acetonitrile/water | Yellow suspension at 25° C. | Insufficient solid |
| (4:1) | Yellow solution at 50° C. | recovered |
| Cumene | Yellow suspension | Form C |
| Nitromethane | Yellow suspension | Form C |

### Example 5.2: Solvent Slurry Screen Using Amorphous Compound 1 Hydrochloride

Amorphous Compound 1 Hydrochloride (20 mg) was charged into 24 crystallization tubes. The relevant solvent (0.5 mL or 1 mL, depending on observed solubility, 25 or 50 volumes) was added. The mixtures were thermally cycled at 25° C. and 50° C. for 4 days. Solids were isolated by filtration at 25° C. All isolated solids were dried in vacuo at 45° C. for 18 hours. Observations and results are summarized in Table 4.

TABLE 4

| | Observations | | |
|---|---|---|---|
| Solvent | 25° C. | 50° C. | XRPD |
| Cyclohexane | Yellow suspension | Yellow suspension | Form C |
| CPME | Yellow suspension | Yellow suspension | Form C |
| Heptane | Yellow suspension | Yellow suspension | Form C |
| TBME | Yellow suspension | Yellow suspension | Form C |
| MIBK | Yellow suspension | Yellow suspension | Form I |
| MEK | Yellow suspension | Yellow suspension | Form I |
| Isopropyl acetate | Yellow suspension | Yellow suspension | Form I |
| Ethyl acetate | Yellow suspension | Yellow suspension | Form I |
| Toluene | Yellow suspension | Yellow suspension | Form C |
| THE | Yellow suspension | Yellow suspension | Form C |
| Methanol | Yellow suspension | Yellow suspension | Form B |
| Ethanol | Yellow suspension | Yellow suspension | Form C |
| IPA | Yellow suspension | Yellow suspension | Form I |
| Acetonitrile | Yellow suspension | Yellow suspension | Form C |
| Chloroform | Yellow suspension | Yellow suspension | Form I |
| Acetone | Yellow suspension | Yellow suspension | Form C |
| 2-Methyl THF | Yellow suspension | Yellow suspension | Form C |
| Water | Yellow solution; | Yellow solution | Mixed phase - Form |
| | Yellow suspension | | B/Form C |
| | following prolonged | | |
| | stir at 25° C. | | |
| Ethanol/1% | Yellow suspension | Yellow suspension | Form C |
| water | | | |
| THF/water (4:1) | Yellow solution | Yellow solution | n/a |
| IPA/water 1% | Yellow suspension | Yellow suspension | Form C |
| 2-Chlorobutane | Yellow suspension | Yellow suspension | Form C |
| Anisole | Yellow suspension | Yellow suspension | Form C |
| Nitromethane | Yellow suspension | Yellow suspension | Form C |

### Example 5.3: Formal Recrystallization Screen Using Amorphous Compound 1 Hydrochloride

Amorphous Compound 1 Hydrochloride (50 mg) was charged into seven crystallization tubes. The relevant solvent (0.5 mL) was added and the resulting mixtures heated to 50° C. All mixtures were suspensions. Deionized water was added into each mixture in 25 μL aliquots to achieve dissolution. The solutions were then cooled gradually to ambient temperature and equilibrated for 18 hours. Where no precipitation occurred, the solvent volume was reduced under a stream of N2 and the resulting solid triturated in the relevant solvent (0.5 mL) to give a suspension. All solids were isolated by filtration. XRPD analysis was collected for damp cakes and following drying of solids at 45° C. for 18 hours. Results are summarized in Table 5.

TABLE 5

| | Volume | | |
|---|---|---|---|
| | of water | | |
| | added | | |
| Solvent | (μL) | Observations | XRPD |
| Methanol | 150 | Remained solution after 18 h | Damp/dry - Form B |
| | | equilibration. Solvent removed and | |
| | | methanol (0.5 mL) added. Suspension | |
| | | matured. | |
| Ethanol | 150 | Remained solution after 18 h | Damp/dry - Low |
| | | equilibration. Solvent removed and | crystallization, similar |
| | | ethanol (0.5 mL) added. Suspension | to Form F |
| | | matured. | |
| IPA | 100 | Suspension after 18 h equilibration. | Damp - Form A |
| | | | Dry - amorphous with |
| | | | Form A characteristics |
| Acetone | 300 | Remained solution after 18 h | Damp - Form A |
| | | equilibration. Solvent removed and | Dry - amorphous with |
| | | acetone (0.5 mL) added. Suspension | Form A characteristics |
| | | matured. | |
| Acetonitrile | 100 | Suspension after 18 h equilibration. | Damp/dry - Form A |
| THF | 150 | Suspension after 18 h equilibration. | Damp/dry - Form A |
| 1,4-Dioxane | 200 | Suspension after 18 h equilibration. | Damp/dry - Form A |

### Example 5.4: Interconversion Study of Form C and Form F

To each of 10 crystallization tubes was added 10 mg Compound 1 Hydrochloride Form C and 10 mg Compound 1 Hydrochloride Form F. The relevant solvent (0.5 mL) was added and the mixtures equilibrated at either 25° C. or 45° C. for 24 hours. The suspensions were filtered to isolate solids which were dried in vacuo at 25° C. for 18 hours. The solids were analyzed using XRPD. Results are summarized in Table 6.

TABLE 6

| Solvent | Temperature | XRPD |
|---|---|---|
| Ethyl acetate | 45° C. | Form C |
| Acetone | 45° C. | Form C |
| IPA | 45° C. | Form C |
| Heptane | 45° C. | Form C |
| Methanol | 45° C. | Form B |
| Ethyl acetate | 25° C. | Form C |
| Acetone | 25° C. | Form C |
| IPA | 25° C. | Form C |
| Heptane | 25° C. | Form C |
| Methanol | 25° C. | Form B |

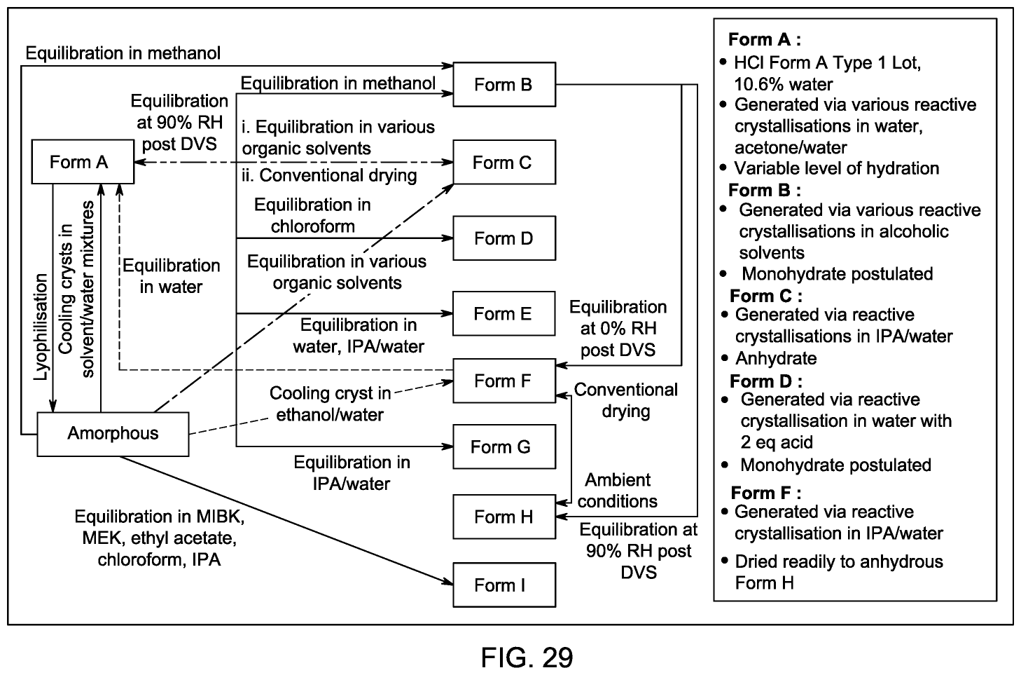

A summary of all polymorph screening is provided in FIG. 29.

### Example 6: Preparation of 1:1 Adduct of Compound 1 and HCl

After it was determined that Compound 1 Hydrochloride Form A prepared as described in Example 2.1.1 contained excess hydrochloric acid, studies were conducted to prepare a 1:1 adduct of Compound 1 and HCl. These experiments and their results are summarized below.

### Example 6.1: Crystallization in Acetone

Compound 1 Hydrochloride Form A (Type 1 Lot) (300 mg) was charged to a crystallization tube with acetone (3 mL, 10 volumes). The suspension was equilibrated at 50° C. Deionized water (800 μL) was added in 100 μL aliquots to achieve dissolution giving a dark yellow solution. The solution was cooled to room temperature and equilibrated for 18 hours. A yellow suspension resulted which was filtered and the solid air dried for 30 minutes. A recovery of 139 mg was recorded. The XRPD pattern of the damp solid indicated Form A. The DSC and TGA showed loss of 11.4% (water content). IC confirmed a 1:1 salt had been isolated, pH 5.1. The solid was subjected to prolonged drying (5 days) at 45° C. and the solids reassessed. XRFD, DSC, and TGA indicated that the solids had fully converted to Form C. Notably, full conversion to Form C was not observed in the drying studies of Example 8 but was observed under the conditions described here.

### Example 6.2: Salt Formation in Acetone (Small Scale)

Compound 1 Free Base (100 mg) was charged to a crystallization tube followed by water (250 μL, 2.5 volumes) and acetone (200 μL, 2 volumes) to give a thick yellow suspension. The suspension was heated to 50° C. Concentrated HCl (15.4 μL, 1 eq) was added which clarified the suspension to a dark yellow solution. The solution was equilibrated at 50° C. for 30 minutes then was cooled to room temperature overnight. No precipitation was evident, so the solvent volume was reduced by 50% under a stream of N2 to give a yellow suspension. Acetone (0.5 mL) was added to give a thick yellow suspension. The suspension was filtered and the isolated solid air dried for 30 minutes to give a yellow solid (75 mg). The XRPD pattern of the damp solid indicated Form A. The DSC and TGA showed loss of 11.4% (water content). A pH assessment indicated pH 5.0 and suggested mono-stoichiometry. The solid was subjected to prolonged drying (5 days) at 45° C. and the solids reassessed. XRFD, DSC, and TGA indicated that the solids had fully converted to Form C. Notably, full conversion to Form C was not observed in the drying studies of Example 8 but was observed under the conditions described here.

### Example 6.3: Salt Formation in Acetone (Large Scale)

Compound 1 Free Base (1.0 g) was charged to reaction tube followed by water (2.5 mL, 2.5 volumes) and acetone (2 mL, 2 volumes) to give a thick yellow suspension. The suspension was heated to 50° C. Concentrated HCl (154 μL, 1 equiv) was added which clarified the suspension to a dark yellow solution. The solution was equilibrated at 50° C. for 30 minutes, then was cooled to room temperature over 5 hours after which time a yellow precipitate had formed. The suspension was filtered and the isolated solid air dried for 30 minutes to give a yellow solid. The solid was dried in vacuo at 25° C. for 18 hours to remove the excess solvent and water from the solid but not fully, so as to prevent significant dehydration leading to partial conversion to Form C. A yellow solid (864 mg, 81% yield) was obtained which was highly crystalline Form A by XRPD with no evidence of Form C. ¹H NMR of the solid confirmed successful salt formation. The solid was then dried for a further 24 hours in vacuo at 45° C. which resulted in complete conversion to Form C. IC analysis confirmed a mono hydrochloride salt.

Two samples of the solid were stored under glass with loose fitting lids on a bench and at 40° C./75% RH for a period of 5 days and re-analyzed by XRPD. In each case, no change from Form C was observed.

### Example 6.4: Salt Formation in Water (1.0 and 1.1 Equiv)

Compound 1 Free Base (50 mg) was charged to 2 crystallization tubes and water (1 mL, 20 volumes) was added to give a thick yellow suspension. The solutions were heated to 50° C. and concentrated hydrochloric acid (A: 7.7 μL, 1 equivalent, B: 8.47 μL, 1.1 equivalents) was added. In both cases, the suspension clarified on addition of the acid to give a dark yellow solution. Mixtures were cooled to 40° C. and various anti-solvents added to see if precipitation could be induced on cooling. This was unsuccessful, so the solvent volume was reduced under a gentle stream of N₂ to give a thick suspension. IPA (1 mL) was added to each suspension and the mixtures equilibrated for 18 hours. The solids were isolated by filtration and dried in vacuo at 45° C. for 4 hours to give bright yellow solids (A: 38 mg, 71%; B: 42 mg, 79%).

The solid isolated from the experiment using one equivalent of concentrated hydrochloric acid (A) was Form A by XRPD, DSC, and TGA. ¹H NMR analysis of the solid suggested a successful salt formation. These results confirmed that the formation of Form A was possible using a controlled stoichiometric equivalent acid charge. Synthesis of Compound 1 Hydrochloride Form A (Type 2 Lot) was scaled-up as described in Example 2 based on this procedure.

The solid isolated from the experiment using 1.1 equivalents of concentrated hydrochloric acid was Form F by XRPD, DSC, and TGA. ¹H NMR analysis of the solid suggested a successful salt formation.

### Example 6.5: Salt Formation in Water (2.0 Equiv)

Compound 1 Free Base (50 mg, 0.092 mmol) was charged to a crystallization tube and water (1 mL) added. The mixture was heated to 50° C. and equilibrated at 50° C. for 30 minutes prior to the addition of concentrated HCl (15.4 μL, 2.0 equiv) which clarified the suspension into a bright yellow solution. The solution was held at temperature for 15 minutes then cooled to ambient temperature over 2 hours maintaining a solution. The solvent volume was reduced to a stirrable minimum using a nitrogen stream and IPA (1 mL) was added to form a mobile suspension that was matured for 18 hours at ambient temperature. Solids were isolated via filtration and dried in vacuo for 18 hours at 45° C. Yield=47.7 mg, 90% based on mono stoichiometry. XRPD, DSC, and TGA confirmed that the isolated solids were Form D.

### Summary of Compound 1 Hydrochloride Form a Stoichiometry Studies

A summary of experiments related to the preparation of a 1:1 adduct of Compound 1 and HCl is provided in Table 7 below.

TABLE 7

| Starting Material | Method of Preparation | Form |
|---|---|---|
| Cmpd 1 Free Base | Acetone/water; 1.2 equiv. conc. HCl; large scale | Form A |
| Cmpd 1 Free Base | DCM (10 vols); 2 equiv. 1M HCl; trituration in | Form B |
| | TBME; 50 mg scale | |
| Cmpd 1 Free Base | DCM/20% THF (10 vols); 1.5 equiv. 1M HCl; | Form B |
| | trituration in heptane; 50 mg scale | |
| Cmpd 1 Free Base | Water (10 vols); 1 equiv. conc. HCl; trituration in | Form A |
| | IPA; 50 mg scale | |
| Cmpd 1 Free Base | Water (10 vols); 1.1 equiv. conc. HCl; trituration in | Form F |
| | IPA; 50 mg scale | |
| Cmpd 1 Free Base | Water (10 vols); 2 equiv. conc. HCl; trituration in | Form D |
| | IPA; 50 mg scale | |
| Cmpd 1 Free Base | Ethanol/methanol (15 vols); 1 equiv. 2M HCl; | Form B |
| | trituration in IPA; 40 mg scale | |
| Cmpd 1 Free Base | IP A/methanol (20 vols); 1 equiv. 2M HCl; 40 mg | Form B |
| | scale | |
| Cmpd 1 Free Base | Methanol (10 vols); 1 equiv. 2M HCl; trituration in | Form B |
| | IPA; 40 mg scale | |
| Cmpd 1 Free Base | IPA/methanol (20 vols); 1 equiv. 2M HCl; | Form C |
| | trituration in IPA; 40 mg scale | |
| Cmpd 1 Free Base | Water; 1 equiv. conc. HCl; trituration in IPA; 1.5 g | Form A |
| | scale | |

### Example 7: DVS Studies of Compound 1 Hydrochloride

### Example 7.1: DVS Studies of Compound 1 Hydrochloride Form B

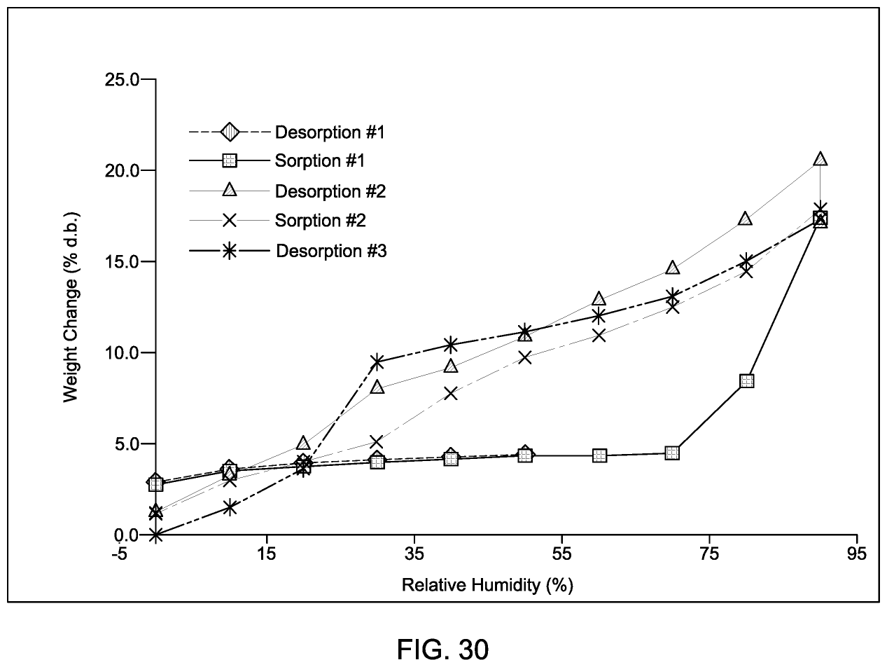

DVS analysis of Form B is shown in FIG. 30. At the beginning of the analysis at 50% RH, the solid showed a moisture content of 4.2% which was consistent with a monohydrate. Minimal loss of water (˜1.5%) was observed on the initial desorption step, which was consistent with the observed TGA. During the initial sorption step, the water content remained essentially constant up to 70% RH, after which the level of water increased rapidly to 17.3%. Water uptake was reversible as relative humidity decreased, although hysteresis was evident and the solid did not appear to revert to its starting form. The solid contained only 1.1% water after the second desorption step indicating that it was no longer a monohydrate. On a second sorption cycle, the solid behaved differently with a more gradual increase in water uptake over the humidity range. The solid demonstrated sponge-like behavior, similar to what was observed with Form A. The water uptake from the second sorption step was reversible, and the solid returned to almost anhydrous with the third desorption step.

XRPD analysis following equilibration at 0% RH indicated that the solid was Form F. XRPD analysis following equilibration at 90% RH indicated that the solid was Form H.

### Example 7.2: DVS Studies of Compound 1 Hydrochloride Form C

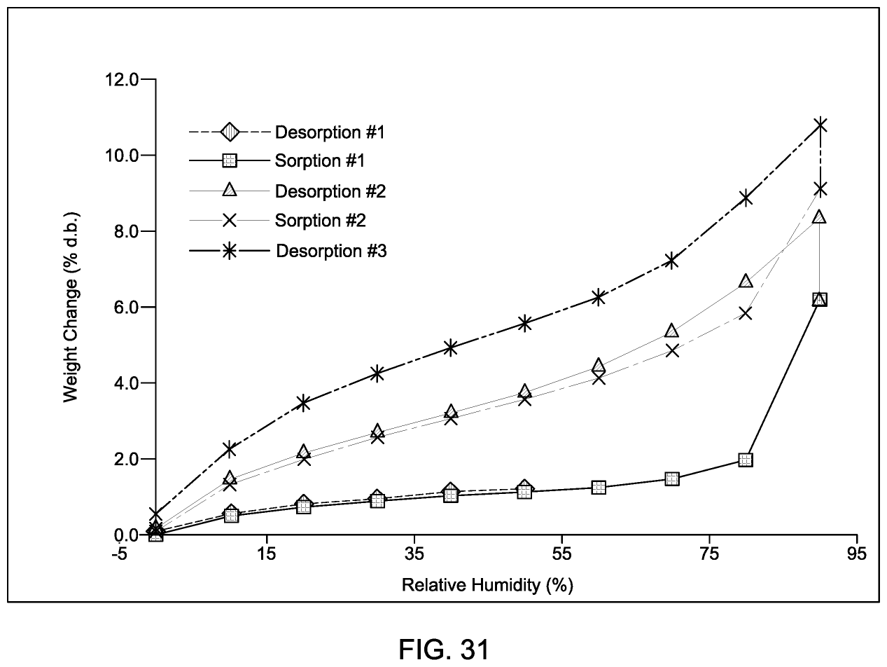

FIG. 31 shows the DVS profile of Compound 1 Hydrochloride Form C (prepared from Form A, Type 1 Lot). During the initial sorption step, the solid absorbs only 1.44% water up to 70% RH, consistent with the observed TGA. Above 70% RH, the solid rapidly absorbs water up to 8.38%, which would be consistent with a trihydrate. The water uptake was reversible as relative humidity decreased. On the second sorption cycle, the solid's composition comprised a higher water content overall which increased gradually up to −10% over the humidity range. Again the water uptake was reversible.

XRPD analysis following equilibration at 0% RH indicated that the solid remained Form C. XRPD analysis following equilibration at 90% RH indicated that the solid was Form A.

### Example 7.3: DVS Studies of Compound 1 Hydrochloride Form A (Type 2 Lot)

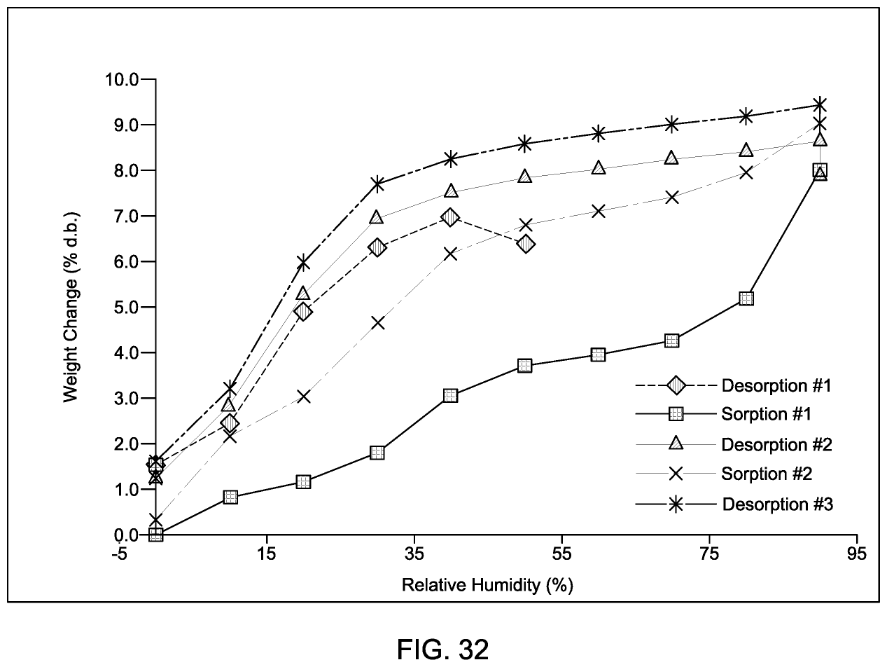

FIG. 32 shows the DVS profile of Compound 1 Hydrochloride Form A (Type 2 Lot). On initial equilibration at 50% RH, the solid contained 6.39% water, indicating a higher order hydrate. (TGA of the solid after drying indicated a water content of only 2.4% which demonstrated that the solid had absorbed water on standing.) Furthermore, a slight uptake of water to −7% is noted following the initial equilibration at 50% RH during the first desorption cycle. This water content decreased as relative humidity decreased in the first desorption cycle. The first sorption cycle results in the solid absorbing up to 8.66% water over the humidity range, consistent with a trihydrate. In comparison to Type 1 Lots of Compound 1 Hydrochloride Form A, which displayed water uptake above 13%, this Type 2 Lot did not absorb as much water, suggesting that excess HCl in the Type 1 Lot may promote increased water sorption at high levels of humidity.

XRPD analysis following equilibration at 0% RH indicated that the solid remained a mixture of Form A and Form C. XRPD analysis following equilibration at 90% RH indicated that the solid was predominantly Form A.

### Example 7.4: DVS Studies of Compound 1 Hydrochloride Form C #2

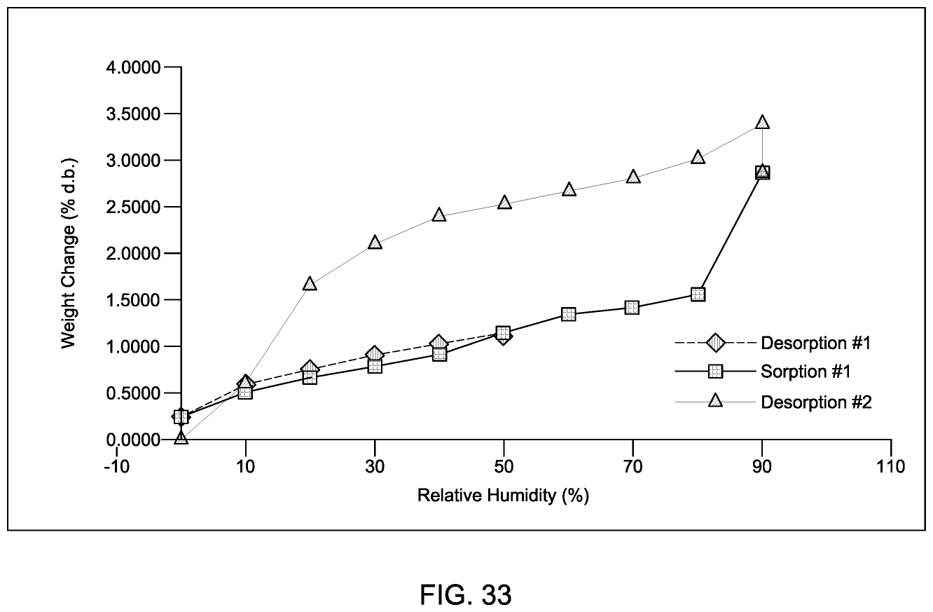

FIG. 33 shows the DVS profile of Compound 1 Hydrochloride Form C (prepared from Form A, Type 2 Lot). The solid absorbed 3.39% water over the humidity range. However, up to 80% RH, the solid absorbed only 1.55% water which increased rapidly above 80% RH. The profile was similar to that of Form C in Example 7.2. These results demonstrate that while conversion between Form A and Form C is possible, kinetics could be slow when starting from Form C substantially free of Form A and/or excess HCl.

XRPD analysis following equilibration at 0% RH and 90% RH indicated that the solid remained Form C.

### Example 8: Drying Studies of Compound 1 Hydrochloride

### Example 8.1: Drying Studies of Form A (Type 2 Lot)

Material for this study was prepared according to the following procedure: Compound 1 Free Base (1.0 g) was mixed with deionized water (15 mL) to yield a suspension at 50° C. To this was added concentrated HCl (1.0 eq., 154 μL) to form a solution at temperature. The solution was aged for 30 minutes and then IPA added (10 mL) and the mixture concentrated in vacuo to give a sticky solid. The solid was triturated and then matured as a mobile suspension in IPA (10 mL) for 18 hours. Filtration in vacuo yielded a damp yellow solid that was air-dried for 30 minutes to yield Compound 1 Hydrochloride Form C as damp solids. The solids were then dried in vacuo at 45° C. to yield Compound 1 Hydrochloride Form C (0.96 g). (Variability in the form of the damp solids was observed across multiple runs, with certain runs producing Compound 1 Hydrochloride Form A or a mixture of Form A and Form C. Such variability may have been related to the relative water content of IPA used in the experiments.)

Compound 1 Hydrochloride Form C prepared as described above was equilibrated in water for 48 hours and readily converted to Form A when analyzed as a damp solid, which confirmed the hypothesis that Form A and Form C could interconvert in the presence/absence of water. Compound 1 Hydrochloride Form A was then subjected to drying under various conditions. Results are summarized in Table 8.

TABLE 8

| | T = 0 | | T = 4 h | | T = 24 h | | T = 48 h | | T = 120 h | |
|---|---|---|---|---|---|---|---|---|---|---|
| | | TGA | | TGA | | TGA | | TGA | | TGA |
| Conditions | XRPD | (%) | XRPD | (%) | XRPD | (%) | XRPD | (%) | XRPD | (%) |
| Dry in | Form | 14.83 | Form | 9.96 | Form A | 7.09 | Form A | 6.61 | Mixed | 3.45 |
| vacuo at | A | | A | | | | | | phase- | |
| 25° C. | | | | | | | | | Forms | |
| | | | | | | | | | A/C | |
| Dry in | Form | 14.83 | Form | 9.89 | Form A | 6.93 | Mixed | 6.63 | Mixed | 2.27 |
| vacuo at | A | | A | | (some | | phase- | | phase- | |
| 45° C. | | | | | Form C) | | Forms | | Forms | |
| | | | | | | | A/C | | A/C | |
| Dry in | Form | 14.83 | Form | 8.93 | Form A | 6.49 | Form A | 6.68 | Form A | 2.17 |
| vacuo at | A | | A | | (some | | (some | | (some | |
| 70° C. | | | | | Form C) | | Form C) | | Form C) | |

### Example 8.2: Drying Studies of Form A (Type 1 Lot)

Drying studies of Compound 1 Hydrochloride Form A (Type 1 Lot) were performed. Results are summarized in Table 9.

TABLE 9

| | T = 0 | | T = 4 h | | T = 24 h | | T = 96 h | | T = 120 h | |
|---|---|---|---|---|---|---|---|---|---|---|
| | | TGA | | TGA | | TGA | | TGA | | TGA |
| Conditions | XRPD | (%) | XRPD | (%) | XRPD | (%) | XRPD | (%) | XRPD | (%) |
| Dry in | — | 9.673 | Form | — | Form A | 11.12 | Form A | 10.14 | Form A | 10.47 |
| vacuo at | | | A | | | | | | | |
| 25° C. | | | | | | | | | | |
| Dry in | — | 9.673 | Form | — | Form A | 9.84 | Form A | 8.71 | Form A | 10.19 |
| vacuo at | | | A | | | | | | | |
| 45° C. | | | | | | | | | | |
| Dry in | — | 9.673 | Form | — | Form A | 10.48 | Form A | 8.19 | Form A | 10.85 |
| vacuo at | | | A | | | | | | | |
| 70° C. | | | | | | | | | | |

### Summary of Drying Studies of Form A (Various Lots)

Variability in the drying profiles of different lots of Compound 1 Hydrochloride were noted, particularly with regards to the transition between Form A and Form C. The variable behavior appeared to depend upon the nature of the input Form A batch (e.g., how it was generated and then conditioned, if at all, prior to drying, and/or if excess hydrochloric acid was present). Table 10 summarizes the results of drying studies.

TABLE 10

| Material | Method of Preparation | XRPD | Impact of drying |
|---|---|---|---|
| Cmpd 1 HCl | As described in Example | Form A | Remained as Form A, only |
| Form A | 2.1.1; contains excess HCl; | | minimal water loss indicated |
| (Type 1 Lot) | generated from salt formation | | by TGA |
| | in acetone/water, 1.2 eq HCl | | |
| Cmpd 1 HCl | Salt formation in water with | Form A | Partial conversion to Form C |
| Form A | conc. HCl generated Form C; | (as | particularly on heating at 25° C. |
| (Type 2 Lot) | Form C slurried in water to | damp | and 45° C.; extended drying |
| | give Form A | solid) | did not result any further |
| | | | change; level of hydration |
| | | | reduced to 2-3% |
| Cmpd 1 HCl | Recrystallization of Cmpd 1 | Form A | Dried fully to Form C; |
| Form A | HCl Form A (Type 1 Lot) in | (as | associated loss of water to give |
| (Type 2 Lot) | acetone/water | damp | anhydrous solid |
| | | solid) | |
| Cmpd 1 HCl | Salt formation in | Form A | Dried fully to Form C; |
| Form A | acetone/water with 1 equiv. | (as | associated loss of water to give |
| (Type 2 Lot) | conc. HCl | damp | anhydrous solid |
| | | solid) | |
| Cmpd 1 HCl | Larger scale salt formation in | Form A | Dried fully to Form C after 24 |
| Form A | acetone/water with 1 equiv. | (as | h at 45° C.; DSC/TGA |
| (Type 2 Lot) | conc. HCl | damp | suggested anhydrate |
| | | solid) | |

### Example 8.3: Drying Studies of Form H

Material for this study was prepared according to the following procedure: Compound 1 Free Base (1.0 g) was suspended in IPA/water (19%) (12.33 volumes) and the suspension heated to 50° C. which thinned the suspension. Concentrated HCl (154 μL, 1 eqiuv) was added which clarified the suspension to a deep orange solution. The mixture was equilibrated for 30 minutes before cooling gradually to room temperature. A thin yellow suspension resulted. The solvent volume was reduced by 50% which thickened the suspension. The suspension was matured overnight, following which the solid was filtered to isolate the solids. The solid was air dried for ˜30 minutes giving a yellow solid (damp mass: 1.24 g; pH measured at 5.0). The XRPD pattern of the damp solid corresponded with Compound 1 Hydrochloride Form H. Analysis of the damp solid by DSC and TGA indicated a water content of 8.2%, which was fairly labile up ˜75° C.

Compound 1 Hydrochloride Form H was then subjected to a drying study under various conditions. Results are summarized in Table 11.

TABLE 11

| | T = 0 | | T = 4 h | | T = 24 h | | T = 96 h | | T = 120 h | |
|---|---|---|---|---|---|---|---|---|---|---|
| | | TGA | | TGA | | TGA | | TGA | | TGA |
| Conditions | XRPD | (%) | XRPD | (%) | XRPD | (%) | XRPD | (%) | XRPD | (%) |
| Dry in | Form H | 8.24 | Form F | 0 | Form F | — | Form F | 0 | Form F | — |
| vacuo at | | | | | | | | | | |
| 25° C. | | | | | | | | | | |
| Dry in | Form H | 8.24 | Form F | 0 | Form F | — | Form F | 0 | Form F | — |
| vacuo at | | | | | | | | | | |
| 45° C. | | | | | | | | | | |
| Dry in | Form H | 8.24 | Form F | 0 | Form F | — | Form F | 0 | Form F | — |
| vacuo at | | | | | | | | | | |
| 70° C. | | | | | | | | | | |

The solids were further subjected to prolonged drying. It was found that once formed, Form F was robust toward further changes at all drying temperatures, providing a highly crystalline solid with no further collapse toward, e.g., Form C.

In an additional experiment, a sample of Form F was left to stand on a bench in a loosened vial for 48 hours. XRPD and TGA indicated that the sample had fully converted into Form H. These results demonstrated that Form F is a metastable anhydrate.

### Example 9: Solid State Stability Studies of Compound 1 Hydrochloride

### Example 9.1: Stability Studies Under Humid Conditions

Stability of Compound 1 Hydrochloride Forms A, B, C, and D was evaluated under humid conditions. Samples of Forms A, B, C, and D were stored at 40° C./75% RH and 25° C./60% RH for a period of two weeks (loosely capped glass vials with approx. 200 mg solid). Solids were then re-analyzed using methods described herein. A summary of the results is provided in Table 12 (after storage at 25° C./60% RH) and Table 13 (after storage at 40° C./75% RH).

TABLE 12

| | | XRPD | | |
|---|---|---|---|---|
| Starting | Appearance | After | | |
| Form | After Storage | Storage | DSC After Storage | TGA After Storage |
| A (Type 1 | Free flowing | Form A | Main melt endotherm at 276.2° C. | Weight reduction of 9.94% up to |
| Lot) | yellow solid | | Sharp endotherm at 114.7° C. | 100° C., further loss of 0.96% |
| | | | | above 200° C. Total water content |
| | | | | of 10.9% |
| A (Type 2 | Free flowing | Form A/C | Main melt endotherm at 278.7° C. | Weight reduction of 7.5% up to |
| Lot) | yellow solid | | Complex series of | 100° C. |
| | | | endotherm events to 150° C. | |
| B | Free flowing | Form B | Main melt endotherm at 283° C. | Weight reduction of 1.69% to 60° C. |
| | yellow solid | | Broad endotherm events | followed by further loss of |
| | | | between 100 and 150° C. | 1.41% post 200° C. |
| C | Free flowing | Form C | Main melt endotherm at 277.5° C. | Weight reduction of 0.89% up to |
| | yellow solid | | | 100° C. followed by further loss of |
| | | | | 0.98% post 200° C. |
| D | Free flowing | Form D | Main melt endotherm at 281.1° C. | Weight reduction of 1.93% up to |
| | yellow solid | | | 100° C. followed by weight loss of |
| | | | | 1.51% post 200° C. |

TABLE 13

| | | XRPD | | |
|---|---|---|---|---|
| Starting | Appearance | After | | |
| Form | After Storage | Storage | DSC After Storage | TGA After Storage |
| A (Type 1 | Yellow solid - | Form A | Main melt endotherm at 272.8° C. | Weight reduction of 10.13% up to |
| Lot) | some | | Complex broad events | 100° C., further loss of 1.05% |
| | agglomeration | | between 90 and 170° C. | above 200° C. Total water content |
| | | | | of 11.18% |
| A (Type 2 | Yellow solid - | amorphous | Main melt endotherm at 280.4° | Weight reduction of 7.03% up to |
| Lot) | some | | C. Complex series of | 100° C. |
| | agglomeration | | endotherm events to 150° C. | |
| B | Free flowing | Form H | Main melt endotherm at 279.5° C. | Weight reduction of 1.0% to 60° C. |
| | yellow solid | | Small endotherm at 251.6° C. | followed by further loss of 1.4% |
| | | | | post 200° C. |
| C | Free flowing | Form C | Main melt endotherm at 277.8° C. | Weight reduction of 0.99% up to |
| | yellow solid | | | 100° C. followed by further loss of |
| | | | | 1.0% post 200° C. |
| D | Free flowing | Form D | Main melt endotherm at 279.5° C. | Weight reduction of 2.24% up to |
| | yellow solid | | | 100° C. followed by weight loss of |
| | | | | 1.68% post 200° C. |

### Example 9.2: Stability Studies Under Desiccant Conditions

Stability of Compound 1 Hydrochloride Forms A and C was evaluated under desiccant conditions. Compound 1 Hydrochloride Form A (Type 1 Lot) and a mixture comprising Compound 1 Hydrochloride Form A (Type 2 Lot) and Form C were stored under desiccant conditions for a two week period. Solids were then re-analyzed using methods described herein. A summary of the results is provided in Table 14.

TABLE 14

| | Appearance | | | |
|---|---|---|---|---|
| Form | After Storage | XRPD | DSC | TGA |
| A (Type 1 | Yellow powder | Form A | Broad endothermic events up | Weight reduction of 5.6% |
| Lot) | | | to 150° C. | |
| | | | Broad main melt endotherm at | |
| | | | 273° C. | |
| A/C (Type | Yellow fluffy | Form A/C | Broad endothermic events up | Weight reduction of 2.3% |
| 2 Lot) | solid | | to 150° C. | |
| | | | Broad main melt endotherm at | |
| | | | 273° C. | |

### Example 9.3: Stability Studies in Sealed Vial

Solid state stability of Compound 1 Hydrochloride Form A was evaluated under various conditions. Compound 1 Hydrochloride Form A (Type 1 Lot) (ca. 200 mg) was placed in a 1.0 mL amber glass HPLC vial to leave minimal headspace and crimp sealed, then heated to a desired temperature. Upon storage for the appropriate period, the entire sample was removed, and the resulting solids assessed via XRFD, HPLC and TGA. Sample preparation for HPLC was conducted using deionized water and a working concentration of 0.5 mg/mL. Preparation of the sample stored at 150° C. required the use of 1:1 THF/water at the same concentration. A control sample was packed and stored in an equivalent manner and tested at T=2 and 4 weeks. Data are summarized in Table 15. Form A was stable at 40° C. and 80° C. for at least up to 4 weeks.

TABLE 15

| | T = 0 | | | T = 1 week | | | T = 2 weeks | | | T = 4 weeks | | |
|---|---|---|---|---|---|---|---|---|---|---|---|---|
| | | TGA | HPLC | | TGA | HPLC | | TGA | HPLC | | TGA | HPLC |
| Condition | XRPD | (%) | (%) | XRPD | (%) | (%) | XRPD | (%) | (%) | XRPD | (%) | (%) |
| Ambient | Form | 12.07 | 99.54 | — | — | — | Form | 11.99 | 99.43 | Form A | 12.15 | 99.53 |
| | A | | | | | | A | | | | | |
| 40° C. | — | — | — | Form | 12.01 | 99.43 | Form | 12.15 | 99.43 | Form A | 11.6 | 99.65 |
| | | | | A | | | A | | | | | |
| 80° C. | — | — | — | New or | 3.76 | 99.53 | New or | 3.71 | 99.45 | New or | 3.35 | 99.59 |
| | | | | mixed | | | mixed | | | mixed | | |
| 150° C. | — | — | — | Low | 1.14 | 36.6 | — | — | — | — | — | — |
| | | | | XT | | | | | | | | |

### Example 10: Salt Screening

### Example 10.1: Salt Formations in Dichloromethane

A salt screen of Compound 1 was conducted to identify and characterize salt forms of Compound 1, other than hydrochloride salt forms. Compound 1 Free Base (50 mg) was charged into 10 crystallization tubes and dichloromethane (0.5 mL, 10 vols) was added. The solutions were heated to 38° C. and the relevant acid (1M solution in methanol, 92.4 μL, 1 equiv.) was added. Mixtures were held at 38° C. for 30 minutes and then allowed to cool to ambient temperature and were equilibrated for 18 hours. For those entries which remained as solutions after equilibration, the solvent was removed with a gentle stream of N2 and the resulting solids triturated in TBME (0.5-1 mL) to give suspensions. The solids were isolated by filtration and dried in vacuo at 45° C. for 18 hours. Six crystalline solids were obtained—from reactions with maleic acid, lactic acid, methanesulfonic acid, L-tartaric acid, acetic acid, and phosphoric acid. Results are summarized in Table 16.

TABLE 16

| | Precipitate | | | | | |
|---|---|---|---|---|---|---|
| | after 18 | Precipitate | | | | |
| | hours at | after | Yield | | | |
| Acid | ambient | work-up | (mg) | XRPD | NMR | DSC |
| Maleic | No | Yes | 55 | Crystalline | 0.8 eq maleic acid | Discrete melt endotherm |
| | | | | | present | at 229° C. |
| Citric | No | Yes | 35 | Amorphous | Citric acid present | Broad endotherm with |
| | | | | | but stoichiometry | peak at 192° C. followed |
| | | | | | unclear | by minor endotherm at |
| | | | | | | 236° C. |
| Succinic | No | Yes | 42 | Low | Succinic acid | Main endotherm at |
| | | | | crystallinity | present but | 224° C. preceded by |
| | | | | | stoichiometry | broad endotherm at |
| | | | | | unclear | 167° C. and minor |
| | | | | | | exotherm at 201° C. |
| Lactic | No | Yes | 48 | Crystalline | Sub- | Complex thermograph |
| | | | | | stoichiometric | with low temperature |
| | | | | | lactic acid | events. Broad main |
| | | | | | | endotherm at 240° C. |
| Methane | No | Yes | 60 | Crystalline | Methane sulfonic | Complex thermograph |
| sulfonic | | | | | acid present | with low temperature |
| | | | | | | events. Small melt |
| | | | | | | endotherm at 234° C. |
| (+)-L- | No | Yes | 63 | Crystalline | 0.75 eq tartaric | Complex thermograph. |
| Tartaric | | | | | acid present | Main melt endo at 229° C. |
| Acetic | No | Yes | 45 | Crystalline | Sub- | Complex thermograph. |
| | | | | | stoichiometric | Bimodal main melt endo |
| | | | | | acetic acid present | with peaks at 247 and |
| | | | | | | 254° C. |
| p-Toluene | No | Yes | 51 | Semi- | 1 eq p- | Broad endotherm at |
| sulfonic | | | | amorphous | toluenesulfonic | 220° C. |
| | | | | | acid present | |
| Sulfuric | Yes | Yes | 23 | Semi- | Salt formation not | Largely featureless |
| | | | | amorphous | evident | thermograph |
| Phosphoric | Yes | Yes | 32 | Crystalline | Relevant proton | Broad main bimodal |
| | | | | | shifts suggest salt | endotherm with main |
| | | | | | formation | peak at 272° C. Broad |
| | | | | | | endo at 131° C. |

### Example 10.2: Salt Formations in Aqueous and Alcoholic Solvents

Compound 1 Free Base (40 mg) was charged into 12 crystallization tubes. The relevant solvent (600 μL) was added, the mixtures heated to 50° C., and the relevant acid added (1 equiv). The mixtures were held at 50° C. for 30 minutes and were then allowed to cool to room temperature and were equilibrated for 18 hours. For those entries that remained as solutions after equilibration, the solvent was removed under a gentle stream of nitrogen and the resulting solids triturated in IPA (0.5-1 mL) to give suspensions. The solids were isolated by filtration and dried in vacuo at 45° C. for 18 hours. Crystalline solid forms of both methanesulfonic acid and p-toluenesulfonic acid were obtained. Results are summarized in Table 17.

TABLE 17

| | | Precipitate | Precipitate | | | | |
|---|---|---|---|---|---|---|---|
| | | after 18 hours | after | Yield | | | |
| Solvent | Acid | at ambient | work-up | (mg) | XRPD | NMR | DSC |
| Ethanol | p-TsOH | No | Yes | 34 | Crystalline | 1 eq p-TsOH | Positive profile- |
| | (1M in | | | | | present | discrete main |
| | MeOH) | | | | | | endotherm at |
| | | | | | | | 267° C. |
| IPA | p-TsOH | Yes | Yes | 30 | Amorphous | 1 eq p-TsOH | Poor thermal |
| | (1M in | | | | | present | profile |
| | MeOH) | | | | | | |
| Methanol | p-TsOH | No | Yes | 33 | Crystalline | 1 eq p-TsOH | Poor thermal |
| | (1M in | | | | (different | present | profile |
| | MeOH) | | | | form) | | |
| Water | p-TsOH | No | Yes | 43 | Amorphous | Sub- | Salt formation |
| | (1M in | | | | | stoichiometric | not supported |
| | MeOH) | | | | | amount of p- | |
| | | | | | | TsOH present | |
| Ethanol | MsOH | Yes | Yes | 42 | Crystalline | 1 eq MsOH | Bimodal |
| | (2M in | | | | | present | endotherm at |
| | MeOH) | | | | | | 245° C. |
| IPA | MsOH | No | Yes | 39 | Crystalline | 1 eq MsOH | Endotherm at |
| | (2M in | | | | | present | 245° C. |
| | MeOH) | | | | | | |
| Methanol | MsOH | Yes | Yes | 32 | Crystalline | 1 eq MsOH | Endotherm at |
| | (2M in | | | | | present | 245° C. |
| | MeOH) | | | | | | |
| Water | MsOH | No | Yes | 29 | Crystalline | 1 eq MsOH | Discrete |
| | (2M in | | | | (different | present but | endotherm at |
| | MeOH) | | | | form) | different to | 300° C. |
| | | | | | | proton shift | |
| Ethanol | HCl | Yes | Yes | 33 | Form B | Salt formation | Main melt |
| | (2M in | | | | | supported | endotherm at |
| | MeOH) | | | | | | 285° C. |
| IPA | HCl | Yes | Yes | 32 | Form B | Salt formation | Main melt |
| | (2M in | | | | | supported | endotherm at |
| | MeOH) | | | | | | 285° C. |
| Methanol | HCl | No | Yes | 42 | Form B | Salt formation | Broad multi- |
| | (2M in | | | | | supported | modal |
| | MeOH) | | | | | | endotherm with |
| | | | | | | | main peak at |
| | | | | | | | 276° C. |
| Water | HCl | No | Yes | 40 | Form C | Salt formation | Broad |
| | (2M in | | | | | supported | endotherm at |
| | MeOH) | | | | | | 273° C. |

### Example 11: Characterization of Salt Forms of Compound 1

### Example 11.1: Compound 1 Maleate

Compound 1 Maleate was obtained according to the following procedure. Compound 1 Free Base (200 mg) was charged into a reaction tube. Dichloromethane (15 volumes) was added and the mixture heated to 50° C. Maleic acid (1M in MeOH, 1.0 equiv) was added and the mixture equilibrated at temperature for 30 minutes before cooling to room temperature. Solvent was partially removed under a stream of N2 and the resulting solid triturated in TBME (2 mL). The suspension matured for 18 hours prior to isolation of the solids by filtration. Solids were dried in vacuo at 45° C. for 18 hours to give Compound 1 Maleate as a bright yellow solid (220 mg, 94% yield). The solid was characterized by XRPD and DSC.

The XRPD pattern of Compound 1 Maleate is shown in FIG. 34, and the corresponding data are summarized below:

| Pos. [°2Th.] | d-spacing [Å] | Rel. Int. [%] |
|---|---|---|
| 5.53 | 15.98 | 4.23 |
| 6.35 | 13.91 | 6.15 |
| 8.12 | 10.88 | 8.51 |
| 9.37 | 9.44 | 29.70 |
| 10.10 | 8.76 | 45.86 |
| 10.89 | 8.12 | 13.69 |
| 11.74 | 7.54 | 19.36 |
| 12.61 | 7.02 | 4.89 |
| 13.00 | 6.81 | 10.28 |
| 14.89 | 5.95 | 23.08 |
| 15.67 | 5.66 | 100.00 |
| 15.91 | 5.57 | 55.60 |
| 16.65 | 5.33 | 22.41 |
| 17.58 | 5.05 | 21.10 |
| 18.08 | 4.91 | 56.95 |
| 18.42 | 4.82 | 46.70 |
| 18.82 | 4.71 | 16.53 |
| 19.43 | 4.57 | 18.93 |
| 19.95 | 4.45 | 12.24 |
| 20.92 | 4.25 | 23.78 |
| 21.76 | 4.08 | 22.47 |
| 22.47 | 3.96 | 23.12 |
| 22.90 | 3.88 | 23.43 |
| 23.81 | 3.74 | 14.70 |
| 24.33 | 3.66 | 59.46 |
| 25.30 | 3.52 | 10.40 |
| 25.83 | 3.45 | 15.92 |
| 26.49 | 3.36 | 12.03 |
| 28.37 | 3.15 | 4.57 |
| 28.87 | 3.09 | 11.00 |
| 29.85 | 2.99 | 7.25 |
| 30.65 | 2.92 | 8.66 |
| 32.16 | 2.78 | 3.06 |
| 34.21 | 2.62 | 4.64 |

As shown by the DSC curve in FIG. 35, the sample displayed an onset of 223.0° C. with a peak at 229.9° C.

### Example 11.2: Compound 1 Mesylate

Compound 1 Mesylate was obtained according to the following procedure. Compound 1 Free Base (200 mg) was charged into a reaction tube, followed by water (3 mL, 15 volumes). The mixture was heated to 50° C. and methanesulfonic acid (2M in MeOH, 185 μL, 1 equivalent) was added. A thinning of the suspension was observed. Methanol (1 mL) was added, which led to clarification of the suspension and gave a deep yellow solution. The solution was cooled to room temperature, and the solvent was removed under a stream of N2. IPA (3 mL) was added and the resulting yellow suspension matured for 18 hours. The solid was dried in vacuo at 45° C. for 18 hours to give Compound 1 Mesylate as a bright yellow solid (161 mg, 68%). The solid was characterized by XRPD and DSC.

The XRPD pattern of Compound 1 Mesylate is shown in FIG. 36, and the corresponding data are summarized below:

| Pos. [°2Th.] | d-spacing [Å] | Rel. Int. [%] |
|---|---|---|
| 5.70 | 15.49 | 4.86 |
| 7.75 | 11.41 | 2.01 |
| 9.53 | 9.28 | 13.92 |
| 9.78 | 9.04 | 18.87 |
| 10.55 | 8.39 | 4.37 |
| 10.93 | 8.09 | 9.18 |
| 11.53 | 7.67 | 12.72 |
| 12.07 | 7.33 | 4.60 |
| 13.05 | 6.78 | 2.10 |
| 13.50 | 6.56 | 4.75 |
| 13.75 | 6.44 | 12.06 |
| 14.08 | 6.29 | 14.47 |
| 14.27 | 6.21 | 7.87 |
| 14.96 | 5.92 | 5.23 |
| 15.87 | 5.58 | 4.12 |
| 16.47 | 5.38 | 23.25 |
| 16.79 | 5.28 | 12.10 |
| 17.36 | 5.11 | 24.80 |
| 17.97 | 4.94 | 8.04 |
| 18.84 | 4.71 | 18.56 |
| 19.18 | 4.63 | 28.59 |
| 19.48 | 4.56 | 64.71 |
| 19.68 | 4.51 | 43.09 |
| 20.12 | 4.41 | 100.00 |
| 20.49 | 4.33 | 15.90 |
| 21.39 | 4.16 | 10.14 |
| 22.10 | 4.02 | 6.34 |
| 22.75 | 3.91 | 13.95 |
| 23.28 | 3.82 | 10.23 |
| 23.97 | 3.71 | 8.61 |
| 24.66 | 3.61 | 11.41 |
| 27.09 | 3.29 | 4.82 |
| 27.75 | 3.22 | 6.28 |
| 29.45 | 3.03 | 1.97 |
| 31.30 | 2.86 | 5.41 |
| 32.81 | 2.73 | 1.13 |

As shown by the DSC curve in FIG. 37, the sample displayed an onset of 296.1° C. with a peak at 300.8° C.

### Example 11.3: Compound 1 Tosylate

Compound 1 Tosylate was obtained according to the following procedure. Compound 1 Free Base (200 mg) was charged into a reaction tube. Ethanol (15 volumes) was added and the mixture heated to 50° C. p-Toluenesulfonic acid (1M in MeOH, 1.0 equiv) was added and the mixture equilibrated at temperature for 30 minutes before cooling to room temperature. Solvent was removed under a stream of N2 and the resulting solid triturated in IPA (2 mL). The suspension matured for 18 hours prior to isolation of the solids by filtration. Solids were dried in vacuo at 45° C. for 18 hours to give Compound 1 Tosylate as a bright yellow solid (260 mg, 99% yield). The solid was characterized by XRPD and DSC.

The XRPD pattern of Compound 1 Tosylate is shown in FIG. 38, and the corresponding data are summarized below:

| Pos. [°2Th.] | d-spacing [Å] | Rel. Int. [%] |
|---|---|---|
| 6.00 | 14.73 | 18.04 |
| 9.72 | 9.10 | 28.77 |
| 10.01 | 8.84 | 62.07 |
| 11.16 | 7.93 | 18.90 |
| 12.18 | 7.27 | 5.30 |
| 12.81 | 6.91 | 36.26 |
| 13.53 | 6.54 | 23.11 |
| 13.95 | 6.35 | 11.46 |
| 16.32 | 5.43 | 12.90 |
| 16.91 | 5.24 | 7.53 |
| 17.58 | 5.05 | 48.46 |
| 18.20 | 4.88 | 63.88 |
| 18.53 | 4.79 | 23.07 |
| 19.44 | 4.57 | 41.35 |
| 19.90 | 4.46 | 100.00 |
| 20.15 | 4.41 | 74.50 |
| 20.66 | 4.30 | 16.00 |
| 21.22 | 4.19 | 10.64 |
| 21.67 | 4.10 | 15.86 |
| 22.35 | 3.98 | 14.91 |
| 23.36 | 3.81 | 8.58 |
| 23.81 | 3.74 | 13.43 |
| 24.09 | 3.70 | 21.96 |
| 24.63 | 3.61 | 9.98 |
| 25.18 | 3.54 | 34.61 |
| 25.42 | 3.50 | 22.81 |
| 25.84 | 3.45 | 12.44 |
| 26.67 | 3.34 | 11.25 |
| 27.32 | 3.26 | 10.91 |
| 29.56 | 3.02 | 3.41 |
| 31.23 | 2.86 | 6.35 |
| 31.95 | 2.80 | 4.62 |
| 33.50 | 2.68 | 4.59 |

As shown by the DSC curve in FIG. 39, the sample displayed an onset of 253.7° C. with a peak at 260.1° C.

### Example 12: Solubility Studies

### Example 12.1: pH-Dependent Solubility

A titration and solubility determination of Compound 1 Hydrochloride Form A (Type 1 Lot) was performed using NaOH over a pH range of 3-8. A solution was produced at 25° C. with magnetic agitation by combining 100 mg Compound 1 Hydrochloride Form A (Type 1 Lot) into 5 mL deionized water to give a concentration of 20 mg/mL within a clean glass vessel. The initial pH reading of pH 3.03 was found to be stable over 10 minutes. The solution was slowly basified using 1.0 N, 0.1 N, and 0.01 N NaOH (with the lower concentrations being used to finely adjust pH close to the end point target pH and avoid overshooting) to achieve pH values of 4, 5, 5.5, 6, 7, and 8. A stable reading was achieved at each pH by equilibration for at least a minimum of 15 minutes and as required to counteract buffering. The concentration of the test solution was lowered at pH 6 from 20 mg/mL to 5 mg/mL with repeat testing at each concentration to illustrate salt impact. Each pH value was allowed to equilibrate to stability prior to solubility evaluation via HPLC assessment of the clarified liquors. Dilution by 100× or 50× into the working range was performed using deionized water. The data is summarized in Table 18. Significant buffering was noted between pH 5.2 and 5.9, with a drift in pH from, e.g., pH 6-5.2, noted across a series of NaOH additions, suggesting a salt break and disproportionation event was taking place, which induced recrystallization and/or precipitation of the poorly soluble free base.

TABLE 18

| | | |
|---|---|---|
| pH | Starting Concentration | Solubility (mg/mL) |

| | | | |
|---|---|---|---|
| 3.03 | 20 | mg/mL | >20 |
| 4.0 | 20 | mg/mL | >20 |
| 5.0 | 20 | mg/mL | 16.0 |
| 5.5 | 20 | mg/mL | 0.7 |
| 6.0 | 20 | mg/mL | 0.06 |
| 6.0 | 5 | mg/mL | 0.06 |
| 7.0 | 5 | mg/mL | 0.05 |
| 8.0 | 5 | mg/mL | 0.001 |

### Example 12.2: Biorelevant Solubility

Due to pH drift in a previous study (see Example 12.1), in order to obtain a more accurate pH solubility evaluation, a study was designed using water, saline, FaSSIF (fasted state simulated intestinal fluid, pH 6.5), FeSSGF (fed state simulated intestinal fluid, pH 5.0), and FaSSGF (fasted state simulated gastric fluid, pH 1.6), which would ensure ending pH and buffer pH remained the same. In order also to avoid pH drift, a lower concentration of 2.5 mg/mL of Compound 1 Hydrochloride Form A (Type 1 Lot) was used. The study design was successful, and all initial pH values were maintained.

Compound 1 Hydrochloride Form A (Type 1 Lot) was dosed in 5 mg portions into the relevant buffers (2 mL) and aqueous solutions and agitated at 37° C. The pH was measured constantly during the addition up to the point of movement in the buffer, at which point additions were halted and the solutions or suspensions matured over 48 hours at 37° C. Samples were taken for solubility evaluation at the specified time-points and clarified through 0.45 micron filters and pH measured on the settled suspensions. Solubility was determined vs a 0.5 mg/mL reference. 35 mg portions were added to FaSSGF and FeSSIF maintaining solutions. FaSSIF tolerated 10 mg/mL prior to movement and a precipitate formed following 5 minutes of agitation that did not change for the duration of the experiment. Salt break to Compound 1 Free Base is consistent with prior findings.

Table 19 summarizes the results from these biorelevant solubility studies. Results for water, saline, and FeSSIF (solubility >50 mg/mL) were consistent with previous studies. Solubility in FaSSGF (start pH 6.5 end 6.1) was depressed and precipitation was observed with solubility at 2.4-5.1 mg/mL over 1-48 h. Solubility in biorelevant media demonstrates good solubility without drastic recrystallization of Compound 1 Free Base observed in, e.g., phosphate buffer alone.

TABLE 19

| | | Solubility | Solubility | Solubility | Solubility |
|---|---|---|---|---|---|
| Buffer (starting | Final | (mg/mL) | (mg/mL) | (mg/mL) | (mg/mL) |
| pH) | pH | T = 1 h | T = 3 h | T = 6 h | T = 24 h |
| Water | 3.04 | >50 | >50 | >50 | >50 |
| 0.9% NaCl | 3.03 | >50 | >50 | >50 | >50 |
| FaSSIF (6.5) | 6.13 | 2.41 | 3.38 | 3.74 | 5.12 |
| FeSSIF (5.0) | 4.98 | >35 | >35 | >35 | >35 |
| FaSSGF (1.6) | 1.65 | >35 | >35 | >35 | >35 |

### Example 12.3: Organic Solubility

Compound 1 Hydrochloride Form A (Type 1 Lot) (ca. 50 mg) was equilibrated in a range of organic solvents in order to collect thermodynamic values for solubility. Solvents were charged in 500 μL aliquots (ca. 10 volumes) to the Compound 1 Hydrochloride Form A (Type 1 Lot) dispensed as a powder (ca. 50 mg) in 16 mm diameter glass reaction tubes until dissolution was achieved or up to 1000 μL (ca. 20 volumes) total volume was charged, with observations noted during dosing. The samples were equilibrated at 25° C. for a minimum of 24 hours with stirrer bead agitation.

Samples which remained as suspensions and contained enough solids were split into two equal portions, with one portion isolated by filtration and the resulting damp solids profiled by XRPD. These solids were then dried in vacuo at 45° C. for ca. 16 hours before further profiling. The filtrates from the solvent suspensions were isolated, diluted by 1 in 50 using acetonitrile/water (1:1), and the Compound 1 content determined by HPLC against a single point standard.

The remaining portion of each suspension was subjected to a heating cycle up to 75° C. with equilibration at 10° C. temperature intervals for a minimum of 30 minutes, before allowing to cool naturally to 40° C., followed by equilibration for ca. 16 hours before continuing to cool naturally (approximately 10° C./hr) to 25° C. Full or partial dissolution was not observed at any temperature (25° C.-75° C.) in any solvent tested.

Solvents employed, solubility observations and XRPD classification for Compound 1 Hydrochloride are summarized in Table 20.

TABLE 20

| | | Solubility at | XRPD after | Observation | XRPD after |
|---|---|---|---|---|---|
| | Volumes | 25° C. | equilibration | after heat/cool | heat/cool |
| Solvent | used | (mg/mL) | at 25° C. | cycle | cycle |
| Methanol | 20 | 4.48 | Form B | Suspension | Mix of Form B and |
| | | | | | Form D |
| Ethanol | 20 | 0.88 | Form C | Suspension | Form C |
| 2-Propanol | 20 | 0.06 | Form C | Suspension | Form C |
| Acetone | 20 | 0.0144 | Form C | Suspension | Form C |
| 2-Butanone | 20 | 0.0049 | Form C | Suspension | Form C |
| MIBK | 20 | 0.0018 | Form C | Suspension | Form C |
| Ethyl acetate | 20 | 0.0018 | Form C | Suspension | Form C |
| Isopropyl acetate | 20 | 0.0028 | Form C | Suspension | Form C |
| n-Butyl acetate | 20 | 0.0007 | Form C | Suspension | Form C |
| Toluene | 20 | 0.0006 | Form A | Suspension | Mix of Form A and |
| | | | | | Form C |
| Anisole | 20 | 0.0081 | Unassigned | Suspension | Unassigned form |
| | | | form | | |
| TBME | 20 | 0.0023 | Form A | Suspension | Mix of Form D and |
| | | | | | unassigned form |
| Diethyl ether | 20 | 0.0030 | Form C | Suspension | Form C |
| Tetrahydrofuran | 20 | 0.0125 | Form C | Suspension | Form C |
| Heptane | 20 | 0.0022 | Form A | Suspension | Form A |
| Dichloromethane | 20 | 1.75 | Form C (low | Suspension | Form C |
| | | | XT) | | |
| Acetonitrile | 20 | 0.17 | Form C | Suspension | Form C |
| DMSO | 20 | 10.82 | Form A | Suspension | Form A |

### Example 12.4: Aqueous Solubility of Salt Forms

Aqueous solubility of certain salt forms was assessed. Each solid (100 mg) was charged into a crystallization tube. Deionized water (1 mL) was added to each tube to give a suspension. The mixtures were equilibrated at 25° C. for 24 hours with sampling after 1 and 4 hours' equilibration. Solids were isolated and XRPD analysis obtained. Filtrates were collected and assessed by HPLC assay for Compound 1 content. Compound 1 Mesylate displayed similar aqueous solubility as Compound 1 Hydrochloride Form A, while the other salt forms were less soluble in water. Results are presented in Table 21.

TABLE 21

| | t = 1 h | | | t = 4 h | | | t = 24 h | | |
|---|---|---|---|---|---|---|---|---|---|
| | | Solubility | | | Solubility | | | Solubility | |
| Salt | pH | mg/mL | XRPD | pH | mg/mL | XRPD | pH | mg/mL | XRPD |
| HCl | 3.34 | 70.37 | Low XT- | n/a | 50.54 | Low XT- | 3.28 | 52.88 | Low XT- |
| (Form | | | Form A | | | Form A | | | Form A |
| A, Type | | | | | | | | | |
| 1 Lot) | | | | | | | | | |
| HCl | 5.22 | 46.11 | Form A | n/a | 43.78 | Form A | 5.25 | 40.58 | Form A |
| (Form | | | | | | | | | |
| A, Type | | | | | | | | | |
| 2 Lot) | | | | | | | | | |
| Free | 10.70 | 0.98 | Different | n/a | 0.10 | Different | 10.51 | 0.07 | Different |
| Base | | | from input | | | from input | | | from input |
| Tosylate | 6.46 | 2.07 | Different | n/a | 2.02 | Different | 6.66 | 2.39 | Different |
| | | | from input | | | from input | | | from input |
| Maleate | 6.35 | 12.50 | Crystalline | n/a | 12.39 | Crystalline | 6.40 | 9.45 | Crystalline |
| | | | (same as | | | (same as | | | (same as |
| | | | input) | | | input) | | | input) |
| Mesylate | 6.01 | 40.23 | Not | n/a | 37.69 | Not | 6.02 | 36.00 | Not |
| | | | collected | | | collected | | | collected |

### Example 13. Solution State Stability of Compound 1 Hydrochloride

The solution state stability of Compound 1 Hydrochloride Form A (Type 1 Lot) was examined under various storage conditions within a suitable glass vessel with stirrer bead agitation at 25° C. Compound 1 Hydrochloride stock solution (100 mg/mL) in HPLC diluent (acetonitrile/water, 1:1) was prepared and 5 mL placed within suitable glass vessels with stirrer bead agitation at 25° C. An aliquot of the appropriate stressing medium (5 mL) was added to achieve a sample concentration of approximately 0.5 mg/mL. The samples were agitated and stored at 25° C. and the solutions assessed for HPLC purity for stability at particular time points. Results are summarized in Table 22.

TABLE 22

| | HPLC (%) | | | | |
|---|---|---|---|---|---|
| Condition | 1 h | 6 h | 24 h | 3 d | 7 d |
| Control | 99.68 | 99.69 | 99.54 | 99.42 | 99.50 |
| (water) | | | | | |
| 1 M HCl | 99.53 | 99.29 | 96.56 | 95.63 | 90.58 |
| 1 M NaOH | 0.49 | 0.65 | — | — | — |
| 5% H2O2 | 80.39 | 46.74 | 0.42 | — | — |

### Example 14. Capsule Formulations

Compound 1 Hydrochloride Form A was formulated in a capsule for oral administration. The capsule formulation included a Size 00 Swedish orange capsule containing Compound 1 (10 mg, 50 mg, or 250 mg) with no excipients. Ingredients of the capsule shell were hypromellose (hydroxypropylmethyl cellulose), iron oxide as a coloring agent, and titanium dioxide as an opacifier.

Capsule formulations were prepared as follows. First, an optional sieving step was performed to deagglomerate the active agent if needed. Then, Compound 1 was filled into HPMC capsules, using either an automated Xelodose machine (e.g., for 10 mg and 50 mg capsules) or a semiautomated process (e.g., for 250 mg capsules). All capsules were polished or dedusted, either by an inline deduster (e.g., for 10 mg and 50 mg capsules) or a separate capsule polisher (e.g., for 250 mg capsules).

### Example 15. Additional Capsule Formluations

Compound 1 Hydrochloride Form A was formulated in a capsule for oral administration. The capsule formulation included a Swedish Orange capsule containing Compound 1 Hydrochloride Form A (100 mg or 200 mg dose) with no excipients. Ingredients of the capsule shell were hypromellose (hydroxypropylmethyl cellulose), iron oxide as a coloring agent, and titanium dioxide as an opacifier. Capsule formulations were prepared as described in Example 14.

1. A solid form of Compound 2: 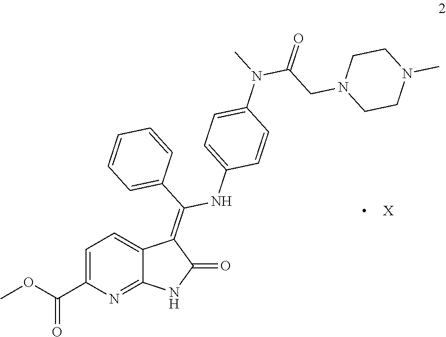
wherein X is hydrochloric acid, maleic acid, methanesulfonic acid, or p-toluenesulfonic acid.

2. The solid form according to claim 1, wherein X is hydrochloric acid.

3. The solid form according to claim 2, wherein the solid form is crystalline.

4. The solid form according to claim 3, wherein the solid form is a hydrate.

5. The solid form according to claim 4, wherein the solid form is characterized by one or more peaks in its XRPD pattern selected from those at about 5.28, 10.63, 11.54, 17.05, and 20.98 degrees 2-theta.

6. The solid form according to claim 4, wherein the solid form is characterized by peaks in its XRPD pattern at about 5.28, 10.63, 11.54, 17.05, and 20.98 degrees 2-theta, corresponding to d-spacing of about 16.74, 8.33, 7.67, 5.20, and 4.23 angstroms, respectively.

7. The solid form according to claim 4, wherein the solid form is characterized by substantially all of the peaks (degrees 2-theta) in its XRPD pattern selected from
°2θ ± 0.2
5.28
5.76
10.63
11.54
12.73
13.13
14.08
15.34
15.64
16.00
16.55
17.05
17.78
18.86
19.08
20.16
20.68
20.98
21.62
22.05
22.82
23.97
24.94
25.23
25.61
26.58
27.01
27.78
29.89
30.51
30.91
31.72
33.39

8. The solid form according to claim 4, wherein the solid form is characterized by one or more of the following:
(i) an XRPD pattern substantially similar to that depicted in FIG. 5;
(ii) a TGA pattern substantially similar to that depicted in FIG. 6; and
(iii) a DSC pattern substantially similar to that depicted in FIG. 6.

9. The solid form according to claim 4, wherein the solid form is characterized by one or more peaks in its XRPD pattern selected from those at about 6.02, 10.07, 10.46, 13.02, and 14.99 degrees 2-theta.

10. The solid form according to claim 4, wherein the solid form is characterized by peaks in its XRPD pattern at about 6.02, 10.07, 10.46, 13.02, and 14.99 degrees 2-theta, corresponding to d-spacing of about 14.68, 8.78, 8.46, 6.80, and 5.91 angstroms, respectively.

11. The solid form according to claim 4, wherein the solid form is characterized by substantially all of the peaks (degrees 2-theta) in its XRPD pattern selected from
°2θ ± 0.2
6.02
10.07
10.46
13.02
14.13
14.99
15.35
16.27
17.65
18.17
18.88
19.63
20.27
20.90
22.41
23.96
24.37
25.45
26.22
26.82
27.90
28.61
29.66
30.30
31.04
31.72
32.91
34.13

12. The solid form according to claim 4, wherein the solid form is characterized by one or more of the following:
(i) an XRPD pattern substantially similar to that depicted in FIG. 7;
(ii) a TGA pattern substantially similar to that depicted in FIG. 8; and
(iii) a DSC pattern substantially similar to that depicted in FIG. 8.

13. The solid form according to claim 3, wherein the solid form is an anhydrate.

14. The solid form according to claim 13, wherein the solid form is characterized by one or more peaks in its XRPD pattern selected from those at about 5.29, 7.44, 9.38, 14.29, and 14.76 degrees 2-theta.

15. The solid form according to claim 13, wherein the solid form is characterized by peaks in its XRPD pattern at about 5.29, 7.44, 9.38, 14.29, and 14.76 degrees 2-theta, corresponding to d-spacing of about 16.70, 11.88, 9.43, 6.20, and 6.00 angstroms, respectively.

16. The solid form according to claim 13, wherein the solid form is characterized by substantially all of the peaks (degrees 2-theta) in its XRPD pattern selected from
°2θ ± 0.2
5.29
7.44
9.38
9.75
10.51
12.97
14.29
14.76
15.66
16.61
16.84
17.26
17.81
18.48
18.87
19.05
19.41
19.90
20.72
21.50
22.38
23.44
24.01
24.73
25.85
26.50
27.44
27.93
28.74
30.04
30.99
32.41
33.89

17. The solid form according to claim 13, wherein the solid form is characterized by one or more of the following:
(i) an XRPD pattern substantially similar to that depicted in FIG. 9;
(ii) a TGA pattern substantially similar to that depicted in FIG. 10; and
(iii) a DSC pattern substantially similar to that depicted in FIG. 10.

18. The solid form according to claim 4, wherein the solid form is characterized by one or more peaks in its XRPD pattern selected from those at about 5.29, 9.37, 9.73, 15.29, 21.60, and 23.71 degrees 2-theta.

19. The solid form according to claim 4, wherein the solid form is characterized by peaks in its XRPD pattern at about 5.29, 9.37, 9.73, 15.29, 21.60, and 23.71 degrees 2-theta, corresponding to d-spacing of about 16.72, 9.44, 9.09, 5.80, 4.12, and 3.75 angstroms, respectively.

20. The solid form according to claim 4, wherein the solid form is characterized by substantially all of the peaks (degrees 2-theta) in its XRPD pattern selected from
°2θ ± 0.2
5.29
9.37
9.73
14.16
15.29
15.99
17.35
18.94
19.30
19.59
19.82
20.26
20.89
21.60
22.99
23.39
23.71
24.94
25.70
26.50
28.23
28.96
30.28
31.27
32.37

21. The solid form according to claim 4, wherein the solid form is characterized by one or more of the following:
(i) an XRPD pattern substantially similar to that depicted in FIG. 11;
(ii) a TGA pattern substantially similar to that depicted in FIG. 12; and
(iii) a DSC pattern substantially similar to that depicted in FIG. 12.

22. The solid form according to claim 4, wherein the solid form is characterized by one or more peaks in its XRPD pattern selected from those at about 4.71, 7.78, 10.30, 13.04, and 15.64 degrees 2-theta.

23. The solid form according to claim 4, wherein the solid form is characterized by peaks in its XRPD pattern at about 4.71, 7.78, 10.30, 13.04, and 15.64 degrees 2-theta, corresponding to d-spacing of about 18.74, 11.37, 8.59, 6.79, and 5.67 angstroms, respectively.

24. The solid form according to claim 4, wherein the solid form is characterized by substantially all of the peaks (degrees 2-theta) in its XRPD pattern selected from
°2θ ± 0.2
4.71
5.61
7.78
9.25
10.30
13.04
14.27
14.88
15.64
16.91
17.67
18.26
20.35
20.69
21.37
22.53
24.20
25.06
25.51
26.31
29.00
29.69
31.28
32.77

25. The solid form according to claim 4, wherein the solid form is characterized by one or more of the following:
(i) an XRPD pattern substantially similar to that depicted in FIG. 13;
(ii) a TGA pattern substantially similar to that depicted in FIG. 14; and
(iii) a DSC pattern substantially similar to that depicted in FIG. 14.

26. The solid form according to claim 13, wherein the solid form is characterized by one or more peaks in its XRPD pattern selected from those at about 6.19, 9.70, 12.09, 16.00, and 20.05 degrees 2-theta.

27. The solid form according to claim 13, wherein the solid form is characterized by peaks in its XRPD pattern at about 6.19, 9.70, 12.09, 16.00, and 20.05 degrees 2-theta, corresponding to d-spacing of about 14.28, 9.12, 7.32, 5.54, and 4.43 angstroms, respectively.

28. The solid form according to claim 13, wherein the solid form is characterized by substantially all of the peaks (degrees 2-theta) in its XRPD pattern selected from
°2θ ± 0.2
5.27
6.19
7.30
9.70
10.65
12.09
12.62
14.41
16.00
16.50
16.83
18.73
19.50
20.05
20.71
21.24
21.49
21.95
22.68
23.08
23.71
24.62
25.74
26.89
27.85
28.92
29.88
30.87
31.53
32.49
33.96

29. The solid form according to claim 13, wherein the solid form is characterized by one or more of the following:
(i) an XRPD pattern substantially similar to that depicted in FIG. 15;
(ii) a TGA pattern substantially similar to that depicted in FIG. 16; and
(iii) a DSC pattern substantially similar to that depicted in FIG. 16.

30. The solid form according to claim 4, wherein the solid form is characterized by one or more of the following:
(i) an XRPD pattern substantially similar to that depicted in FIG. 17;
(ii) a TGA pattern substantially similar to that depicted in FIG. 18; and
(iii) a DSC pattern substantially similar to that depicted in FIG. 18.

31. The solid form according to claim 4, wherein the solid form is characterized by one or more peaks in its XRPD pattern selected from those at about 5.52, 9.60, 11.46, 13.66, and 20.13 degrees 2-theta.

32. The solid form according to claim 4, wherein the solid form is characterized by peaks in its XRPD pattern at about 5.52, 9.60, 11.46, 13.66, and 20.13 degrees 2-theta, corresponding to d-spacing of about 16.00, 9.21, 7.72, 6.48, and 4.41 angstroms, respectively.

33. The solid form according to claim 4, wherein the solid form is characterized by substantially all of the peaks (degrees 2-theta) in its XRPD pattern selected from
°2θ ± 0.2
5.52
9.60
10.85
11.46
12.93
13.66
15.60
16.07
16.52
18.39
19.36
20.13
21.60
22.29
23.12
24.16
25.35
26.97
30.03
31.95
32.71

34. The solid form according to claim 4, wherein the solid form is characterized by one or more of the following:
(i) an XRPD pattern substantially similar to that depicted in FIG. 19;
(ii) a TGA pattern substantially similar to that depicted in FIG. 20; and
(iii) a DSC pattern substantially similar to that depicted in FIG. 20.

35. The solid form according to claim 13, wherein the solid form is characterized by one or more peaks in its XRPD pattern selected from those at about 9.42, 12.24, 17.40, 17.60, 17.80, and 18.22 degrees 2-theta.

36. The solid form according to claim 13, wherein the solid form is characterized by one or more peaks in its XRPD pattern selected from those at about 9.42, 12.24, 17.40, 17.60, 17.80, and 18.22 degrees 2-theta, corresponding to d-spacing of about 9.39, 7.23, 5.10, 5.04, 4.98, and 4.87 angstroms, respectively.

37. The solid form according to claim 13, wherein the solid form is characterized by substantially all of the peaks (degrees 2-theta) in its XRPD pattern selected from
°2θ ± 0.2
5.60
9.42
12.24
13.44
15.42
16.06
16.59
17.40
17.60
17.80
18.22
18.95
19.45
20.58
22.85
23.26
23.78
24.24
24.81
25.61
26.48
28.68
29.58

38. The solid form according to claim 13, wherein the solid form is characterized by one or more of the following:
(i) an XRPD pattern substantially similar to that depicted in FIG. 21;
(ii) a TGA pattern substantially similar to that depicted in FIG. 22; and
(iii) a DSC pattern substantially similar to that depicted in FIG. 22.

39. The solid form according to claim 1, wherein X is maleic acid.

40. The solid form according to claim 39, wherein the solid form is crystalline.

41. The solid form according to claim 40, wherein the solid form is characterized by one or more peaks in its XRPD pattern selected from those at about 9.37, 10.10, 11.74, 14.89, 15.67, 15.91, 18.08, and 18.42 degrees 2-theta.

42. The solid form according to claim 40, wherein the solid form is characterized by peaks in its XRPD pattern at about 9.37, 10.10, 11.74, 14.89, 15.67, 15.91, 18.08, and 18.42 degrees 2-theta, corresponding to d-spacing of about 9.44, 8.76, 7.54, 5.95, 5.66, 5.57, 4.91, and 4.82 angstroms, respectively.

43. The solid form according to claim 40, wherein the solid form is characterized by substantially all of the peaks in its XRPD pattern selected from
°2θ ± 0.2
5.53
6.35
8.12
9.37
10.10
10.89
11.74
12.61
13.00
14.89
15.67
15.91
16.65
17.58
18.08
18.42
18.82
19.43
19.95
20.92
21.76
22.47
22.90
23.81
24.33
25.30
25.83
26.49
28.37
28.87
29.85
30.65
32.16
34.21

44. The solid form according to claim 40, wherein the solid form is characterized by one or more of the following:
(i) an XRPD pattern substantially similar to that depicted in FIG. 34; and
(ii) a DSC pattern substantially similar to that depicted in FIG. 35.

45. The solid form according to claim 1, wherein X is methanesulfonic acid.

46. The solid form according to claim 45, wherein the solid form is crystalline.

47. The solid form according to claim 46, wherein the solid form is characterized by one or more peaks in its XRPD pattern selected from those at about 9.53, 9.78, 16.47, 17.36, 19.48, and 20.12 degrees 2-theta.

48. The solid form according to claim 46, wherein the solid form is characterized by peaks in its XRPD pattern at about 9.53, 9.78, 16.47, 17.36, 19.48, and 20.12 degrees 2-theta, corresponding to d-spacing of about 9.28, 9.04, 5.38, 5.11, 4.56, and 4.41 angstroms, respectively.

49. The solid form according to claim 46, wherein the solid form is characterized by substantially all of the peaks in its XRPD pattern selected from
°2θ ± 0.2
5.70
7.75
9.53
9.78
10.55
10.93
11.53
12.07
13.05
13.50
13.75
14.08
14.27
14.96
15.87
16.47
16.79
17.36
17.97
18.84
19.18
19.48
19.68
20.12
20.49
21.39
22.10
22.75
23.28
23.97
24.66
27.09
27.75
29.45
31.30
32.81

50. The solid form according to claim 46, wherein the solid form is characterized by one or more of the following:
(i) an XRPD pattern substantially similar to that depicted in FIG. 36; and
(ii) a DSC pattern substantially similar to that depicted in FIG. 37.

51. The solid form according to claim 1, wherein X is p-toluenesulfonic acid.

52. The solid form according to claim 51, wherein the solid form is crystalline.

53. The solid form according to claim 52, wherein the solid form is characterized by one or more peaks in its XRPD pattern selected from those at about 9.72, 10.01, 11.16, 12.81, 17.58, and 18.20 degrees 2-theta.

54. The solid form according to claim 52, wherein the solid form is characterized by peaks in its XRPD pattern at about 9.72, 10.01, 11.16, 12.81, 17.58, and 18.20 degrees 2-theta, corresponding to d-spacing of about 9.10, 8.84, 7.93, 6.91, 5.05, 4.88 angstroms, respectively.

55. The solid form according to claim 52, wherein the solid form is characterized by substantially all of the peaks in its XRPD pattern selected from
°2θ ± 0.2
6.00
9.72
10.01
11.16
12.18
12.81
13.53
13.95
16.32
16.91
17.58
18.20
18.53
19.44
19.90
20.15
20.66
21.22
21.67
22.35
23.36
23.81
24.09
24.63
25.18
25.42
25.84
26.67
27.32
29.56
31.23
31.95
33.50

56. The solid form according to claim 52, wherein the solid form is characterized by one or more of the following:
(i) an XRPD pattern substantially similar to that depicted in FIG. 38; and
(ii) a DSC pattern substantially similar to that depicted in FIG. 39.

57. A crystalline solid form of Compound 1:

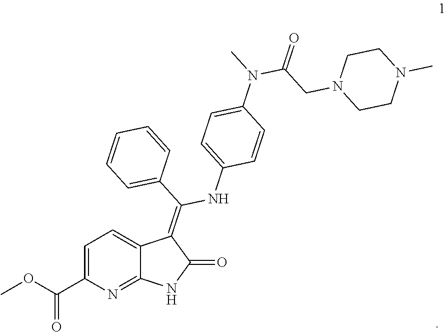

58. The solid form according to claim 57, wherein the solid form is characterized by one or more peaks in its XRPD pattern selected from those at about 9.12, 12.31, 17.58, 18.12, and 19.19 degrees 2-theta.

59. The solid form according to claim 57, wherein the solid form is characterized by peaks in its XRPD pattern at about 9.12, 12.31, 17.58, 18.12, and 19.19 degrees 2-theta, corresponding to d-spacing of about 9.70, 7.19, 5.04, 4.90, and 4.63 angstroms, respectively.

60. The solid form according to claim 57, wherein the solid form is characterized by substantially all of the peaks (degrees 2-theta) in its XRPD pattern selected from
°2θ ± 0.2
5.55
6.81
9.12
10.56
12.31
13.78
15.35
16.54
17.58
18.12
19.19
20.77
22.78
24.34
26.51
29.45
30.24

61. The solid form according to claim 57, wherein the solid form is characterized by an XRPD pattern substantially similar to that depicted in FIG. 25.

62. The solid form according to claim 57, wherein the solid form is characterized by one or more peaks in its XRPD pattern selected from those at about 7.69, 13.39, 14.64, 18.98, and 20.93 degrees 2-theta.

63. The solid form according to claim 57, wherein the solid form is characterized by peaks in its XRPD pattern at about 7.69, 13.39, 14.64, 18.98, and 20.93 degrees 2-theta, corresponding to d-spacing of about 11.50, 6.61, 6.05, 4.68, and 4.24 angstroms, respectively.

64. The solid form according to claim 57, wherein the solid form is characterized by substantially all of the peaks (degrees 2-theta) in its XRPD pattern selected from
°2θ ± 0.2
5.60
7.69
8.76
9.47
10.96
13.39
14.64
16.25
16.66
16.88
17.41
18.98
19.98
20.93
22.03
23.20
23.79
26.07
27.25
27.87
31.51

65. A pharmaceutical composition comprising a solid form according to any one of claims 1-64 and a pharmaceutically acceptable carrier.

66. A capsule composition comprising a solid form according to any one of claims 1-64.

67. The capsule composition of claim 66, wherein the capsule composition comprises 100 mg Compound 1.

68. The capsule composition of claim 66, wherein the capsule composition comprises 200 mg Compound 1.

69. The capsule composition of any one of claims 66-68, wherein the capsule composition comprises Compound 1 with no excipients.

70. A method of treating a disease associated with activity of platelet-derived growth factor receptors (PDGFR) and/or a vascular endothelial growth factor receptor, the method comprising administering to a patient in need thereof a solid form according to any of claims 1-64.

71. A method of treating a fibrotic disease or disorder, the method comprising administering to a patient in need thereof a solid form according to any of claims 1-64.

72. The method according to claim 71, wherein the fibrotic disease or disorder is fibrosis.

73. The method according to claim 72, wherein the fibrosis is pulmonary fibrosis.

74. The method according to claim 73, wherein the pulmonary fibrosis is idiopathic pulmonary fibrosis (IPF).

75. The method according to claim 72, wherein the fibrosis is hepatic fibrosis.

76. The method according to claim 72, wherein the fibrosis is renal fibrosis.

77. The method according to claim 71, wherein the fibrotic disease or disorder is nephrotic syndrome or a disease, disorder, or condition associated with nephrotic syndrome.

78. The method according to claim 77, wherein the fibrotic disease or disorder is focal and segmental glomerulosclerosis (FSGS).

79. The method according to claim 71, wherein the fibrotic disease or disorder is primary glomerular disease.

80. The method according to claim 71, wherein the fibrotic disease or disorder is primary proteinuric kidney disease.

81. The method according to claim 71, wherein the fibrotic disease or disorder is membranous nephropathy.

82. The method according to claim 71, wherein the fibrotic disease or disorder is IgA nephropathy.

83. The method according to claim 71, wherein the fibrotic disease or disorder is FSGS.

84. The method according to claim 72, wherein the fibrosis is dermal fibrosis.

85. The method according to claim 72, wherein the fibrosis is gastrointestinal fibrosis.
